(19) United States
(12) Patent Application Publication
(10) Pub. No.: US 2026/0306607 A1
(43) Pub. Date: Oct. 8, 2026

(54) Vaccine Compositions

(71) Applicant: Seqirus Inc., Holly Springs, NC (US)

(72) Inventors: Douglas Widman, Holly Springs, NC (US); Palaniappan Ramanathan, Holly Springs, NC (US)

(73) Assignee: Seqirus Inc., Holly Springs, NC (US)

(21) Appl. No.: 19/481,466

(22) PCT Filed: May 6, 2024
(86) PCT No.: PCT/IB2024/054366

§ 371 Date: Nov. 4, 2025

Related U.S. Application Data
(60) Provisional application No. 63/500,377, filed on May 5, 2023.
(60) Provisional application No. 63/500,378, filed on May 5, 2023.

(51) Int. Cl. A61K 39/12; A61K 39/00; A61K 39/39; A61P 37/04

(52) U.S. Cl. CPC A61K 39/12; A61K 39/39; A61P 37/04; A61K 2039/53

(57) ABSTRACT

The present disclosure relates to RNAs and other polynucleotides encoding an antigen, which can be used in vaccine compositions. In some examples, the RNA also encodes a chemoattractant, such as chemerin, C-X-C motif chemokine ligand 9 (CX-CL9), C-X-C motif chemokine ligand 10 (CXCL10) or C-X-C motif chemokine ligand 11 (CXCL11). The present disclosure also relates to use of the RNAs and compositions in methods of inducing an immune response in a subject.

### RELATED APPLICATION DATA

The present application is a national phase entry pursuant to 35 U.S.C. § 371 of International Application No. PCT/IB2024/054366, filed May 6, 2024, which claims priority to U.S. Patent Application No. 63/500,378, filed May 5, 2023, and U.S. Application No. 63/500,377, filed May 5, 2023, the entire contents of which are hereby incorporated by reference for any purpose.

### SEQUENCE LISTING

The present application contains a Sequence Listing, which has been submitted electronically in XML format and is hereby incorporated by reference in its entirety. Said XML copy, created on Jun. 1, 2026, is named “537612USP.xml” and is 86,772 bytes in size.

### FIELD

The present disclosure relates to RNA and/or protein based immunopotentiating agents for use in vaccine compositions.

### BACKGROUND

Bacterial, viral, and parasitic infections are wide spread in humans and animals. Diseases caused by these infectious agents are often resistant to antimicrobial pharmaceutical therapy, leaving no effective means of treatment. Consequently, a vaccinology approach is increasingly used to control infectious disease. A whole infectious pathogen can be made suitable for use in a vaccine formulation after chemical inactivation or appropriate genetic manipulation. Alternatively, a protein subunit of the pathogen can be expressed in a recombinant expression system and purified for use in a vaccine formulation. Vaccines can be made more efficacious by enhancing immunogenicity and/or slowing release of the antigens from the injection site.

Traditional vaccines are generally composed of a crude preparation of inactivated or killed or modified live pathogenic microorganisms. The impurities associated with these cultures of pathological microorganisms may act as an adjuvant or immunopotentiating agent to enhance the immune response. However, the immunity invoked by vaccines that use homogeneous preparations of pathological microorganisms or purified protein subunits as antigens is often poor. The addition of certain exogenous materials that act as adjuvants or immunopotentiating agents therefore becomes necessary. Further, synthetic and subunit vaccines are expensive to produce, and the addition of an immunopotentiating agent potentially permits the use of a smaller dose of antigen to stimulate a similar immune response, thereby reducing the production cost of the vaccine. Thus, the effectiveness of some injectable medicinal agents may be significantly increased when the agent is combined with an immunopotentiating agent.

Immunopotentiating agents are generally utilised to increase the magnitude or function of the antibody response, increase cell mediated immunity, induce mucosal immunity, and/or reduce antigen dose. For example, the first immunopotentiating agent reported was Freund's Complete Adjuvant (FCA) which contains a water-in-oil emulsion and extracts of mycobacterium. FCA is however poorly tolerated and can cause uncontrolled inflammation. Since the discovery of FCA over 80 years ago, efforts have been made to reduce the unwanted side effects of immunopotentiating agents. Other materials used as immunopotentiating agents include metallic oxides (e.g., aluminum hydroxide), alum, inorganic chelates of salts, gelatins, various paraffin-type oils, synthesized resins, alginates, mucoid and polysaccharide compounds, caseinates, and blood-derived substances such as fibrin clots. While these materials are generally efficacious at stimulating the immune system, they have been unsuitable for use in vaccines due to adverse effects in the host such as production of abscesses, organ damage, carcinogenicity and allergenic responses. Undesirable pharmaceutical properties such as rapid dispersion or poor control of dispersion from the injection site, or swelling of the material have also been reported.

A skilled person will therefore understand that there remains a need for the development of new immunopotentiating agents for use in vaccine compositions.

### SUMMARY

The present disclosure is based on the inventors' finding that combining an antigen with a chemoattractant in an immunogenic composition, such as a vaccine, can enhance a subject's immune response to the immunogenic composition.

The findings by the inventors therefore provide basis for a RNA comprising a nucleotide sequence encoding a chemoattractant and a nucleotide sequence encoding an antigen of interest. The findings by the inventors also provide basis for immunogenic compositions comprising (i) a RNA comprising a nucleotide sequence encoding an antigen of interest and a nucleotide sequence encoding a chemoattractant and (ii) a chemoattractant polypeptide. The findings by the inventors also provide basis for immunogenic compositions comprising (i) a RNA comprising a nucleotide sequence encoding an antigen of interest and (ii) a chemoattractant polypeptide. Furthermore, the findings by the inventors provide basis for methods of treating or preventing or delaying progression of a disease or disorder (e.g., a disease caused by a respiratory viral infection, such as influenza, a SARS-COV-2 infection, COVID-19 or ARDS) in a subject using the immunogenic compositions of the disclosure.

Accordingly, the present disclosure provides a polynucleotide comprising a first nucleotide sequence encoding an antigen operably linked to a regulatory element, and a second nucleotide sequence encoding a chemoattractant operably linked to a regulatory element.

The present disclosure also provides a RNA comprising a first nucleotide sequence encoding an antigen operably linked to a regulatory element, and a second nucleotide sequence encoding a chemoattractant operably linked to a regulatory element.

The present disclosure also provides a cRNA comprising a first nucleotide sequence encoding an antigen operably linked to a regulatory element, and a second nucleotide sequence encoding a chemoattractant operably linked to a regulatory element.

The present disclosure also provides a self-replicating RNA comprising a first nucleotide sequence encoding an antigen operably linked to a regulatory element, and a second nucleotide sequence encoding a chemoattractant operably linked to a regulatory element.

In an example, the self-replicating RNA comprises in 5′ to 3′ order:
a) the nucleotide sequence encoding an antigen operably linked to a regulatory element; and
b) the nucleotide sequence encoding a chemoattractant operably linked to a regulatory element.

In another example, the self-replicating RNA comprises in 5′ to 3′ order:
a) the nucleotide sequence encoding a chemoattractant operably linked to a regulatory element; and
b) the nucleotide sequence encoding an antigen operably linked to a regulatory element.

In an example, the self-replicating RNA comprises in 5′ to 3′ order:
a) the nucleotide sequence encoding an antigen operably linked to a SG promoter; and
b) the nucleotide sequence encoding a chemoattractant operably linked to a regulatory element selected from the group consisting of a SG promoter and an internal ribosome entry site (IRES).

In another example, the self-replicating RNA comprises in 5′ to 3′ order:
a) the nucleotide sequence encoding a chemoattractant operably linked to a regulatory element selected from the group consisting of a SG promoter and an internal ribosome entry site (IRES); and
b) the nucleotide sequence encoding an antigen operably linked to a SG promoter.

In an example, the self-replicating RNA comprises in 5′ to 3′ order:
a) the nucleotide sequence encoding a chemoattractant operably linked to a SG promoter; and
b) the nucleotide sequence encoding an antigen operably linked to a regulatory element selected from the group consisting of a SG promoter and an internal ribosome entry site (IRES).

In an example, the self-replicating RNA comprises in 5′ to 3′ order:
a) the nucleotide sequence encoding a chemoattractant operably linked to a SG promoter; and
b) the nucleotide sequence encoding an antigen operably linked to a SG promoter.

In an example, the chemoattractant is selected from the group consisting of chemerin, C-X-C motif chemokine ligand 9 (CXCL9), C-X-C motif chemokine ligand 10 (CXCL10) and C-X-C motif chemokine ligand 11 (CXCL11).

In some examples, the chemoattractant is chemerin.

In some examples, the self-replicating RNA comprises in 5′ to 3′ order:
a) the nucleotide sequence encoding an antigen operably linked to a regulatory element; and
b) the nucleotide sequence encoding a chemerin operably linked to a regulatory element.

In some examples, the self-replicating RNA comprises in 5′ to 3′ order:
a) the nucleotide sequence encoding a chemerin operably linked to a regulatory element; and
b) the nucleotide sequence encoding an antigen operably linked to a regulatory element.

In some examples, the self-replicating RNA comprises in 5′ to 3′ order:
a) the nucleotide sequence encoding an antigen operably linked to a SG promoter; and
b) the nucleotide sequence encoding a chemerin operably linked to a regulatory element selected from the group consisting of a SG promoter and an internal ribosome entry site (IRES).

In some examples, the self-replicating RNA comprises in 5′ to 3′ order:
a) the nucleotide sequence encoding a chemerin operably linked to a SG promoter; and
b) the nucleotide sequence encoding an antigen operably linked to a regulatory element selected from the group consisting of a SG promoter and an internal ribosome entry site (IRES).

In some examples, the self-replicating RNA comprises in 5′ to 3′ order:
a) the nucleotide sequence encoding a chemerin operably linked to a SG promoter; and
b) the nucleotide sequence encoding an antigen operably linked to a SG promoter.

Thus, in an example of the disclosure, there is provided a polynucleotide comprising a first nucleotide sequence encoding an antigen operably linked to a regulatory element, and a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element.

The present disclosure also provides a RNA comprising a first nucleotide sequence encoding an antigen operably linked to a regulatory element, and a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element.

The present disclosure also provides a cRNA comprising a first nucleotide sequence encoding an antigen operably linked to a regulatory element, and a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element.

The present disclosure also provides a self-replicating RNA comprising a first nucleotide sequence encoding an antigen operably linked to a regulatory element, and a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element.

In an example, the regulatory element is selected from the group consisting of a subgenomic (SG) promoter, an internal ribosome entry site (IRES) and a Kozak consensus sequence or a combination thereof. In an example, the regulatory element is a SG promoter.

In an example, the nucleotide sequence encoding the chemoattractant is operably linked to the same regulatory element as the nucleotide sequence encoding the antigen.

In an example, the polynucleotide, RNA, cRNA or self-replicating RNA comprises a second antigen. In another example, polynucleotide, RNA, cRNA or self-replicating RNA comprises second and third antigens.

The present disclosure also provides a polynucleotide comprising: a) a first nucleotide sequence encoding a first polypeptide of interest; and b) a second nucleotide sequence encoding a chemoattractant operably linked to a regulatory element selected from the group consisting of a subgenomic (SG) promoter and an internal ribosome entry site (IRES).

In one example, the polynucleotide comprises, in order from 5′ to 3′: a) a first nucleotide sequence encoding a first polypeptide of interest; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to a regulatory element selected from the group consisting of a SG promoter and an IRES.

The present disclosure also provides a polynucleotide comprising: a) a first nucleotide sequence encoding a first antigen of interest; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding an antigen of interest; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding an antigen of interest operably linked to a SG promoter; and b) a second nucleotide sequence encoding a chemerin operably linked to a SG promoter.

In one example, the polynucleotide comprises, in order from 5′ to 3′: a) a first nucleotide sequence encoding a chemerin operably linked to a SG promoter; and b) a second nucleotide sequence encoding an antigen of interest operably linked to a SG promoter.

In an example, the polynucleotide is RNA or DNA. In one example, the RNA is messenger RNA (mRNA). In one example, the mRNA is conventional mRNA (cRNA) or self-replicating RNA.

Accordingly, the present disclosure provides a RNA comprising: a) a first nucleotide sequence encoding a first antigen of interest; and b) a second nucleotide sequence encoding a chemoattractant operably linked to a regulatory element selected from the group consisting of a SG promoter and an IRES.

In one example, the RNA comprises a) a first nucleotide sequence encoding a first antigen of interest; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES

In one example, the RNA comprises, in order from 5′ to 3′: a) a first nucleotide sequence encoding a first antigen of interest; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant optionally selected from the group consisting of chemerin, CXCL9, CXCL10 and CXCL11; and b) a second nucleotide sequence encoding an antigen of interest operably linked to a regulatory element selected from the group consisting of a SG promoter and an IRES.

The present disclosure also provides a cRNA comprising: a) a first nucleotide sequence encoding a first antigen of interest; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a first antigen of interest; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a chemoattractant; and b) a second nucleotide sequence encoding an antigen of interest operably linked to a regulatory element selected from the group consisting of a SG promoter and an IRES.

The present disclosure further provides a self-replicating RNA comprising: a) a first nucleotide sequence encoding a first antigen of interest; and b) a second nucleotide sequence encoding a chemoattractant operably linked to a regulatory element selected from the group consisting of a SG promoter and an IRES.

In one example, the self-replicating RNA comprises: a) a first nucleotide sequence encoding a first antigen of interest; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the self-replicating RNA comprises, in order from 5′ to 3′: a) a first nucleotide sequence encoding a first antigen of interest; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding an antigen of interest; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding an antigen of interest operably linked to a SG promoter; and b) a second nucleotide sequence encoding a chemerin operably linked to a SG promoter.

In one example, the self-replicating RNA comprises, in order from 5′ to 3′: a) a first nucleotide sequence encoding a chemerin operably linked to a SG promoter; and b) a second nucleotide sequence encoding an antigen of interest operably linked to a SG promoter.

In one example, the first nucleotide sequence is operably linked to a regulatory element. In another example, the regulatory element is operably linked to the 5′ end of the first nucleotide sequence. In one example, the regulatory element is selected from the group consisting of a Kozak consensus sequence, an IRES, a SG promoter and combinations thereof. For example, the regulatory element is a Kozak consensus sequence. In another example, the regulatory element is an IRES. In an example, the nucleotide sequence encoding the chemoattractant is operably linked to an IRES located 3′ to the nucleotide sequence encoding the chemoattractant. In another example, the regulatory element is a SG promoter.

In one example, the Kozak consensus sequence comprises or consists of GCCACC. In one example, the Kozak consensus sequence consists of GCCACC. In one example, the Kozak consensus sequence comprises GCCACC. For example, the Kozak consensus sequence is ACCATGG.

In one example, the Kozak consensus sequence comprises or consists of ACCATGG. In one example, the Kozak consensus sequence consists of ACCATGG. In one example, the Kozak consensus sequence comprises ACCATGG. For example, the Kozak consensus sequence of ACCATGG.

The present disclosure provides a polynucleotide comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a SG promoter; and b) a second nucleotide sequence encoding a chemerin operably linked to a SG promoter.

In one example, the polynucleotide comprises, in order from 5′ to 3′: a) a first nucleotide sequence encoding a chemerin operably linked to a operably linked to a SG promoter; and b) a second nucleotide sequence encoding an antigen of interest operably linked to a SG promoter.

The present disclosure provides a RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant operably linked to a regulatory element selected from the group consisting of a SG promoter and an IRES.

In one example, the RNA comprises: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemerin operably linked to a regulatory element selected from the group consisting of a SG promoter and an IRES.

In one example, the RNA comprises, in order from 5′ to 3′: a) a first nucleotide sequence encoding a chemerin operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a SG promoter; and b) a second nucleotide sequence encoding a chemerin operably linked to a SG promoter.

In one example, the RNA comprises, in order from 5′ to 3′: a) a first nucleotide sequence encoding a chemerin operably linked to a SG promoter f; and b) a second nucleotide sequence encoding an antigen of interest operably linked to a SG promoter.

The present disclosure provides a cRNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a chemoattractant operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemerin operably linked to a regulatory element selected from the group consisting of a SG promoter and an IRES.

In one example, the cRNA comprises, in order from 5′ to 3′: a) a first nucleotide sequence encoding a chemerin operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the cRNA comprises, in order from 5′ to 3′: a) a first nucleotide sequence encoding a first antigen of interest optionally operably linked to an IRES; and b) a second nucleotide sequence encoding a chemerin operably linked to an IRES.

In one example, the cRNA comprises, in order from 5′ to 3′: a) a first nucleotide sequence encoding a chemerin optionally operably linked to an IRES; and b) a second nucleotide sequence encoding an antigen of interest operably linked to an IRES.

The present disclosure also provides a composition comprising a cRNA encoding a first antigen of interest and a cRNA encoding a chemerin.

The present disclosure provides a self-replicating RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant operably linked to a regulatory element selected from the group consisting of a SG promoter and an IRES.

In one example, the self-replicating RNA comprises: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a SG promoter; and b) a second nucleotide sequence encoding a chemerin operably linked to a SG promoter.

In one example, the self-replicating RNA comprises, in order from 5′ to 3′: a) a first nucleotide sequence encoding a chemerin operably linked to a SG promoter; and b) a second nucleotide sequence encoding an antigen of interest operably linked to a SG promoter.

In one example, the first nucleotide sequence is operably linked to a Kozak consensus sequence.

In one example, the first nucleotide sequence is operably linked to a Kozak consensus sequence and a SG promoter. For example, the Kozak consensus sequence is operably linked to the 5′ end of the SG promoter which is operably linked to the 5′ end of the first nucleotide sequence.

In one example, the first nucleotide sequence is operably linked to a Kozak consensus sequence and an IRES. For example, the Kozak consensus sequence is operably linked to the 5′ end of the IRES which is operably linked to the 5′ end of the first nucleotide sequence.

In one example, the first nucleotide sequence is operably linked to a SG promoter.

In one example, the first nucleotide sequence is operably linked to an IRES.

The present disclosure provides a polynucleotide comprising: a) a first nucleotide sequence encoding a first antigen of interest operably linked to a Kozak consensus sequence; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to a Kozak consensus sequence; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin operably linked to a Kozak consensus sequence; and b) a second nucleotide sequence encoding an antigen of interest operably linked to a regulatory element selected from the group consisting of a SG promoter and an IRES.

The present disclosure provides a polynucleotide comprising: a) a first nucleotide sequence encoding a first antigen of interest operably linked to a Kozak consensus sequence and a SG promoter; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence and a SG promoter; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to a Kozak consensus sequence and a SG promoter; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence and a SG promoter; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin operably linked to a Kozak consensus sequence and a SG promoter; and b) a second nucleotide sequence encoding an antigen of interest operably linked to a regulatory element selected from the group consisting of a SG promoter and an IRES.

The present disclosure provides a polynucleotide comprising: a) a first nucleotide sequence encoding a first antigen of interest operably linked to a Kozak consensus sequence and an IRES; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence and an IRES; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to a Kozak consensus sequence and an IRES; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a Kozak consensus sequence and an IRES; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin operably linked to a Kozak consensus sequence and an IRES; and b) a second nucleotide sequence encoding an antigen of interest operably linked to a regulatory element selected from the group consisting of a SG promoter and an IRES.

The present disclosure provides a polynucleotide comprising: a) a first nucleotide sequence encoding a first antigen of interest operably linked to a SG promoter; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest operably linked to a SG promoter; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to a SG promoter; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to a SG promoter; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin operably linked to a SG promoter; and b) a second nucleotide sequence encoding an antigen of interest operably linked to a regulatory element selected from the group consisting of a SG promoter and an IRES.

The present disclosure provides a polynucleotide comprising: a) a first nucleotide sequence encoding a first antigen of interest operably linked to an IRES; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a first antigen of interest operably linked to an IRES; and b) a second nucleotide sequence encoding a chemoattractant operably linked to a regulatory element selected from the group consisting of a SG promoter and an IRES.

In one example, the polynucleotide comprises, in order from 5′ to 3′: a) a first nucleotide sequence encoding a chemoattractant operably linked to an IRES; and b) a second nucleotide sequence encoding an antigen of interest operably linked to a regulatory element selected from the group consisting of a SG promoter and an IRES.

In one example, the polynucleotide comprises, in order from 5′ to 3′: a) a first nucleotide sequence encoding a first antigen of interest operably linked to an IRES; and b) a second nucleotide sequence encoding a chemerin operably linked to a regulatory element selected from the group consisting of a SG promoter and an IRES.

In one example, the polynucleotide comprises, in order from 5′ to 3′: a) a first nucleotide sequence encoding a chemerin operably linked to an IRES; and b) a second nucleotide sequence encoding an antigen of interest operably linked to a regulatory element selected from the group consisting of a SG promoter and an IRES.

The present disclosure provides a RNA comprising: a) a first nucleotide sequence encoding a first antigen of interest operably linked to a Kozak consensus sequence; and b) a second nucleotide sequence encoding a chemoattractant operably linked to a regulatory element selected from the group consisting of a SG promoter and an IRES.

In one example, the RNA comprises a) a first nucleotide sequence encoding a first antigen of interest operably linked to a Kozak consensus sequence; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11), operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence; and b) a second nucleotide sequence encoding a chemerin operably linked to a regulatory element selected from the group consisting of a SG promoter and an IRES.

In one example, the RNA comprises, in order from 5′ to 3′: a) a first nucleotide sequence encoding a chemerin operably linked to a Kozak consensus sequence; and b) a second nucleotide sequence encoding an antigen of interest operably linked to a regulatory element selected from the group consisting of a SG promoter and an IRES.

The present disclosure provides a RNA comprising: a) a first nucleotide sequence encoding a first antigen of interest operably linked to a Kozak consensus sequence and a SG promoter; and b) a second nucleotide sequence encoding a chemoattractant operably linked to a regulatory element selected from the group consisting of a SG promoter and an IRES.

In one example, the RNA comprises a) a first nucleotide sequence encoding a first antigen of interest operably linked to a Kozak consensus sequence and a SG promoter; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence and a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and a SG promoter; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence and a SG promoter; and b) a second nucleotide sequence encoding a chemerin operably linked to a regulatory element selected from the group consisting of a SG promoter and an IRES.

In one example, the RNA comprises, in order from 5′ to 3′: a) a first nucleotide sequence encoding a chemerin operably linked to a Kozak consensus sequence and a SG promoter; and b) a second nucleotide sequence encoding an antigen of interest operably linked to a regulatory element selected from the group consisting of a SG promoter and an IRES.

The present disclosure provides a RNA comprising: a) a first nucleotide sequence encoding a first antigen of interest operably linked to a Kozak consensus sequence and an IRES; and b) a second nucleotide sequence encoding a chemoattractant operably linked to a regulatory element selected from the group consisting of a SG promoter and an IRES.

In one example, the RNA comprises a) a first nucleotide sequence encoding a first antigen of interest operably linked to a Kozak consensus sequence and an IRES; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence and an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and an IRES; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a Kozak consensus sequence and an IRES; and b) a second nucleotide sequence encoding a chemerin operably linked to a regulatory element selected from the group consisting of a SG promoter and an IRES.

In one example, the RNA comprises, in order from 5′ to 3′: a) a first nucleotide sequence encoding a chemerin operably linked to a Kozak consensus sequence and an IRES; and b) a second nucleotide sequence encoding an antigen of interest operably linked to a regulatory element selected from the group consisting of a SG promoter and an IRES.

The present disclosure provides a RNA comprising: a) a first nucleotide sequence encoding a first antigen of interest operably linked to a SG promoter; and b) a second nucleotide sequence encoding a chemoattractant operably linked to a regulatory element selected from the group consisting of a SG promoter and an IRES.

In one example, the RNA comprises a) a first nucleotide sequence encoding a first antigen of interest operably linked to a SG promoter; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the RNA comprises, in order from 5′ to 3′: a) a first nucleotide sequence encoding a first antigen of interest operably linked to a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a SG promoter; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to a SG promoter; and b) a second nucleotide sequence encoding a chemerin operably linked to a regulatory element selected from the group consisting of a SG promoter and an IRES.

In one example, the RNA comprises, in order from 5′ to 3′: a) a first nucleotide sequence encoding a chemerin operably linked to a SG promoter; and b) a second nucleotide sequence encoding an antigen of interest operably linked to a regulatory element selected from the group consisting of a SG promoter and an IRES.

The present disclosure provides a RNA comprising: a) a first nucleotide sequence encoding a first antigen of interest operably linked to an IRES; and b) a second nucleotide sequence encoding a chemoattractant operably linked to a regulatory element selected from the group consisting of a SG promoter and an IRES.

In one example, the RNA comprises: a) a first nucleotide sequence encoding a first antigen of interest operably linked to an IRES; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the RNA comprises, in order from 5′ to 3′: a) a first nucleotide sequence encoding a first antigen of interest operably linked to an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to an IRES; and b) a second nucleotide sequence encoding an antigen of interest operably linked to a regulatory element selected from the group consisting of a SG promoter and an IRES.

In one example, the RNA comprises, in order from 5′ to 3′: a) a first nucleotide sequence encoding a first antigen of interest operably linked to an IRES; and b) a second nucleotide sequence encoding a chemerin operably linked to a regulatory element selected from the group consisting of a SG promoter and an IRES.

In one example, the RNA comprises, in order from 5′ to 3′: a) a first nucleotide sequence encoding a chemerin operably linked to an IRES; and b) a second nucleotide sequence encoding an antigen of interest operably linked to a regulatory element selected from the group consisting of a SG promoter and an IRES.

The present disclosure provides a cRNA comprising: a) a first nucleotide sequence encoding a first antigen of interest operably linked to a Kozak consensus sequence; and b) a second nucleotide sequence encoding a chemoattractant operably linked to a regulatory element selected from the group consisting of a SG promoter and an IRES In one example, the cRNA comprises, in order from 5′ to 3′: a) a first nucleotide sequence encoding a first antigen of interest operably linked to a Kozak consensus sequence; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding an adjuvant operably linked to a Kozak consensus sequence; and b) a second nucleotide sequence encoding a chemoattractant of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence; and b) a second nucleotide sequence encoding a chemerin operably linked to a regulatory element selected from the group consisting of a SG promoter and an IRES.

In one example, the cRNA comprises, in order from 5′ to 3′: a) a first nucleotide sequence encoding an adjuvant operably linked to a Kozak consensus sequence; and b) a second nucleotide sequence encoding a chemerin operably linked to a regulatory element selected from the group consisting of a SG promoter and an IRES.

The present disclosure provides a cRNA comprising: a) a first nucleotide sequence encoding a first antigen of interest operably linked to a Kozak consensus sequence and a SG promoter; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence and a SG promoter; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a chemoattractant operably linked to a Kozak consensus sequence and a SG promoter; and b) a second nucleotide sequence encoding a chemoattractant of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence and a SG promoter; and b) a second nucleotide sequence encoding a chemerin operably linked to a regulatory element selected from the group consisting of a SG promoter and an IRES.

In one example, the cRNA comprises, in order from 5′ to 3′: a) a first nucleotide sequence encoding a chemerin operably linked to a Kozak consensus sequence and a SG promoter; and b) a second nucleotide sequence encoding a chemoattractant of interest operably linked to a regulatory element selected from the group consisting of a SG promoter and an IRES.

The present disclosure provides a cRNA comprising: a) a first nucleotide sequence encoding a first antigen of interest operably linked to a Kozak consensus sequence and an IRES; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence and an IRES; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a chemoattractant operably linked to a Kozak consensus sequence and an IRES; and b) a second nucleotide sequence encoding an antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a Kozak consensus sequence and an IRES; and b) a second nucleotide sequence encoding a chemerin operably linked to a regulatory element selected from the group consisting of a SG promoter and an IRES.

In one example, the cRNA comprises, in order from 5′ to 3′: a) a first nucleotide sequence encoding a chemerin operably linked to a Kozak consensus sequence and an IRES; and b) a second nucleotide sequence encoding an antigen of interest operably linked to a regulatory element selected from the group consisting of a SG promoter and an IRES.

The present disclosure provides a cRNA comprising: a) a first nucleotide sequence encoding a first antigen of interest operably linked to a SG promoter; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a first antigen of interest operably linked to a SG promoter; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a chemoattractant operably linked to a SG promoter; and b) a second nucleotide sequence encoding an antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to a SG promoter; and b) a second nucleotide sequence encoding a chemerin operably linked to a regulatory element selected from the group consisting of a SG promoter and an IRES.

In one example, the cRNA comprises, in order from 5′ to 3′: a) a first nucleotide sequence encoding a chemerin operably linked to a SG promoter; and b) a second nucleotide sequence encoding an antigen of interest operably linked to a regulatory element selected from the group consisting of a SG promoter and an IRES.

The present disclosure provides a cRNA comprising: a) a first nucleotide sequence encoding a first antigen of interest operably linked to an IRES; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a first antigen of interest operably linked to an IRES; and b) a second nucleotide sequence encoding a chemoattractant operably linked to a regulatory element selected from the group consisting of a SG promoter and an IRES.

In one example, the cRNA comprises, in order from 5′ to 3′: a) a first nucleotide sequence encoding a chemoattractant operably linked to an IRES; and b) a second nucleotide sequence encoding an antigen of interest operably linked to a regulatory element selected from the group consisting of a SG promoter and an IRES.

In one example, the cRNA comprises, in order from 5′ to 3′: a) a first nucleotide sequence encoding a first antigen of interest operably linked to an IRES; and b) a second nucleotide sequence encoding a chemerin operably linked to a regulatory element selected from the group consisting of a SG promoter and an IRES.

In one example, the cRNA comprises, in order from 5′ to 3′: a) a first nucleotide sequence encoding a chemerin operably linked to an IRES; and b) a second nucleotide sequence encoding an antigen of interest operably linked to a regulatory element selected from the group consisting of a SG promoter and an IRES.

The present disclosure provides a self-replicating RNA comprising: a) a first nucleotide sequence encoding a first antigen of interest operably linked to a Kozak consensus sequence; and b) a second nucleotide sequence encoding a chemoattractant operably linked to a regulatory element selected from the group consisting of a SG promoter and an IRES.

In one example, the self-replicating RNA comprises a) a first nucleotide sequence encoding a first antigen of interest operably linked to a Kozak consensus sequence; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11), operably linked to a regulatory element selected from the group consisting of a SG promoter and an IRES.

In one example, the self-replicating RNA comprises, in order from 5′ to 3′:
a) a first nucleotide sequence encoding a first antigen of interest operably linked to a Kozak consensus sequence; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a Kozak consensus sequence; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin operably linked to a Kozak consensus sequence; and b) a second nucleotide sequence encoding an antigen of interest operably linked to a regulatory element selected from the group consisting of a SG promoter and an IRES.

The present disclosure provides a self-replicating RNA comprising: a) a first nucleotide sequence encoding a first antigen of interest operably linked to a Kozak consensus sequence and a SG promoter; and b) a second nucleotide sequence encoding a chemoattractant operably linked to a regulatory element selected from the group consisting of a SG promoter and an IRES.

In one example, the self-replicating comprises a) a first nucleotide sequence encoding a first antigen of interest operably linked to a Kozak consensus sequence and a SG promoter; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a Kozak consensus sequence and a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and a SG promoter; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a Kozak consensus sequence and a SG promoter; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin operably linked to a Kozak consensus sequence and a SG promoter; and b) a second nucleotide sequence encoding an antigen of interest operably linked to a regulatory element selected from the group consisting of a SG promoter and an IRES.

The present disclosure provides a self-replicating RNA comprising: a) a first nucleotide sequence encoding a first antigen of interest operably linked to a Kozak consensus sequence and an IRES; and b) a second nucleotide sequence encoding a chemoattractant operably linked to a regulatory element selected from the group consisting of a SG promoter and an IRES.

In one example, the self-replicating RNA comprises a) a first nucleotide sequence encoding a first antigen of interest operably linked to a Kozak consensus sequence and an IRES; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a Kozak consensus sequence and an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and an IRES; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a Kozak consensus sequence and an IRES; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin operably linked to a Kozak consensus sequence and an IRES; and b) a second nucleotide sequence encoding an antigen of interest operably linked to a regulatory element selected from the group consisting of a SG promoter and an IRES.

The present disclosure provides a self-replicating RNA comprising: a) a first nucleotide sequence encoding a first antigen of interest operably linked to a SG promoter; and b) a second nucleotide sequence encoding a chemoattractant operably linked to a regulatory element selected from the group consisting of a SG promoter and an IRES.

In one example, the self-replicating RNA comprises a) a first nucleotide sequence encoding a first antigen of interest operably linked to a SG promoter; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a SG promoter; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to a SG promoter; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin operably linked to a SG promoter; and b) a second nucleotide sequence encoding an antigen of interest operably linked to a regulatory element selected from the group consisting of a SG promoter and an IRES.

The present disclosure provides a self-replicating RNA comprising: a) a first nucleotide sequence encoding a first antigen of interest operably linked to an IRES; and b) a second nucleotide sequence encoding a chemoattractant operably linked to a regulatory element selected from the group consisting of a SG promoter and an IRES.

In one example, the self-replicating RNA comprises: a) a first nucleotide sequence encoding a first antigen of interest operably linked to an IRES; and b) a second nucleotide sequence encoding a chemoattractant selected from the group consisting of chemerin, C-X-C motif chemokine ligand 9 (CXCL9), C-X-C motif chemokine ligand 10 (CXCL10) and C-X-C motif chemokine ligand 11 (CXCL11) operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to an IRES; and b) a second nucleotide sequence encoding an antigen of interest operably linked to a regulatory element selected from the group consisting of a SG promoter and an IRES.

In one example, the self-replicating RNA comprises, in order from 5′ to 3′: a) a first nucleotide sequence encoding a first antigen of interest operably linked to an IRES; and b) a second nucleotide sequence encoding a chemerin operably linked to a regulatory element selected from the group consisting of a SG promoter and an IRES.

In one example, the self-replicating RNA comprises, in order from 5′ to 3′: a) a first nucleotide sequence encoding a chemerin operably linked to an IRES; and b) a second nucleotide sequence encoding an antigen of interest operably linked to a regulatory element selected from the group consisting of a SG promoter and an IRES.

In one example, the polynucleotide is a bicistronic RNA. For example, the polynucleotide is a bicistronic cRNA. In one example, the cRNA is a bicistronic cRNA. In another example, the polynucleotide is a bicistronic self-replicating mRNA. For example, the self-replicating RNA is a bicistronic self-replicating RNA.

In one example, the second nucleotide sequence is operably linked to an IRES.

In one example, the second nucleotide sequence is operably linked to a SG promoter.

In one example, the polynucleotide is a multicistronic RNA. For example, the polynucleotide is a multicistronic cRNA. For example, the cRNA is a multicistronic cRNA. In another example, the polynucleotide is a multicistronic self-replicating mRNA. For example, the self-replicating RNA is a multicistronic self-replicating mRNA.

In one example, the SG promoter is a native SG promoter. For example, a native SG promoter is a promoter that is native to the RNA virus from which it is derived and/or based on (e.g., an alphavirus). In one example, the native SG promoter is a native alphavirus SG promoter.

In one example, the SG promoter is a minimal SG promoter or an extended SG promoter.

In one example, the SG promoter is a minimal SG promoter. In one example, the native SG promoter is a minimal SG promoter. For example, the minimal SG promoter is the minimal sequence required for initiation of transcription. In one example, the minimal native SG promoter is 49 nucleotides in length. In one example, the minimal SG promoter is 49 nucleotides in length.

In one example, the minimal native SG promoter is encoded by a sequence comprising or consisting of a sequence set forth in SEQ ID NO: 32. In one example, the minimal SG promoter is encoded by a sequence comprising or consisting of a sequence set forth in SEQ ID NO: 32.

In one example, the SG promoter is an extended SG promoter. In one example, the native SG promoter is an extended SG promoter. For example, the extended SG promoter is extended at the 5′ end with nucleotides occurring in a sequence encoding a non-structural protein (e.g., NSP4) of the RNA virus (e.g., an alphavirus). In one example, the extended SG promoter is extended at the 5′ end with nucleotides occurring in a sequence encoding an alphavirus NSP4. The addition of nucleotides to the 5′ end of the SG promoter sequence did not interfere with expression of the non-structural protein and viral replicase, e.g., alphavirus NSP4.

In one example, the SG promoter is extended at the 5′ end by 51 or fewer nucleotides occurring in a sequence encoding a non-structural protein (e.g., an alphavirus NSP4). In one example, the extended SG promoter is a minimal SG promoter extended at the 5′ end by no more than 51 nucleotides occurring in a sequence encoding a non-structural protein (e.g., an alphavirus NSP4). In one example, the extended SG promoter is encoded by a sequence comprising or consisting of a sequence set forth in SEQ ID NO: 32 extended at the 5′ end by no more than 51 nucleotides occurring in a sequence encoding a non-structural protein (e.g., an alphavirus NSP4). For example, the extended SG promoter is no more than 100 nucleotides in length. In one example, the extended SG promoter is encoded by a sequence comprising or consisting of nucleotides 2 to 101 of SEQ ID NO: 36.

In one example, the SG promoter is extended at the 5′ end by about 5 nucleotides to about 20 nucleotides, for example by about 5 nucleotides, or about 10 nucleotides, or about 12, or about 15 nucleotides, or about 20 nucleotides, occurring in a sequence encoding a non-structural protein (e.g., an alphavirus NSP4). In another example, the SG promoter is extended at the 5′ end by about 20 to about 35 nucleotides, for example, by about 25 nucleotides or about 27 nucleotides, or about 30 nucleotides, or about 35 nucleotides, occurring in a sequence encoding a non-structural protein (e.g., an alphavirus NSP4).

In one example, the SG promoter is extended at the 5′ end by about 12 nucleotides occurring in a sequence encoding a non-structural protein (e.g., an alphavirus NSP4). In one example, the extended SG promoter is encoded by a sequence set forth in SEQ ID NO: 32 extended at the 5′ end by 12 nucleotides occurring in a sequence encoding a non-structural protein (e.g., an alphavirus NSP4). For example, the extended SG promoter is no more than 61 nucleotides in length. In one example, the extended SG promoter is encoded by a sequence comprising or consisting of nucleotides 41 to 101 of SEQ ID NO: 36. In another example, the extended SG promoter is encoded by a sequence comprising or consisting of a sequence set forth in SEQ ID NO: 33.

In one example, the SG promoter is extended at the 5′ end by about 31 nucleotides occurring in a sequence encoding a non-structural protein (e.g., an alphavirus NSP4). In one example, the extended SG promoter is encoded by a sequence set forth in SEQ ID NO: 32 extended at the 5′ end by 31 nucleotides occurring in a sequence encoding a non-structural protein (e.g., an alphavirus NSP4). For example, the extended SG promoter is no more than 80 nucleotides in length. In one example, the extended SG promoter is encoded by a sequence comprising or consisting of nucleotides 22 to 101 of SEQ ID NO: 36. In another example, the extended SG promoter is encoded by a sequence comprising or consisting of a sequence set forth in SEQ ID NO: 34.

In one example, the extended SG promoter comprises a repeat sequence corresponding to nucleotides 66 to 75 of SEQ ID NO: 36. For example, the extended SG promoter is encoded by a sequence comprising nucleotides 50 to 75 of SEQ ID NO: 36 and nucleotides 66 to 101 of SEQ ID NO: 36. For example, the extended SG promoter is encoded by a sequence set forth in SEQ ID NO: 46.

In some examples, the extended SG promoter is encoded by a sequence set forth in any one of SEQ ID NOs: 33, 34, 36 or 46.

In some examples, the extended SG promoter is encoded by a sequence set forth in any one of SEQ ID NOs: 33, 34, 36, 46, 51 or 52. In some examples, the extended SG promoter is encoded by a sequence set forth in SEQ ID NO: 51 or 52.

In one example, the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (eIF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1a (FGF1A), GTX, or a combination thereof.

In one example, the IRES is a wild-type IRES derived from encephalomyocarditis virus (EMCV). For example, the wild-type EMCV IRES comprises a sequence set forth in SEQ ID NO: 35.

In one example, the first and/or second nucleotide sequence and/or the one or more additional nucleotide sequences are codon optimized.

In one example, the G/C content of the first and/or second nucleotide sequence and/or the one or more additional nucleotide sequences are modified.

In one example, the G/C content of the first and/or second nucleotide sequence and/or the one or more additional nucleotide sequences are increased by at least 5% compared to the G/C content of the unmodified sequence. For example, the G/C content of the first and/or second nucleotide sequence and/or the one or more additional nucleotide sequences are increased by at least 10%, or 15%, or 20%, or 25%, or 30%, or 35%, or 40% compared to the G/C content of the unmodified sequence.

In one example, the polynucleotide comprises at least one chemically modified nucleotide.

In one example, the chemically modified nucleotide is selected from the group consisting of N6,2′-O-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), 1-methylpseudouridine (m1ψ), and combinations thereof. For example, the chemically modified nucleotide is N6,2′-O-dimethyl-adenosine (m6Am). For example, the chemically modified nucleotide is 5-methyluridine (m5U). For example, the chemically modified nucleotide is N4-acetylcytidine (ac4C). For example, the chemically modified nucleotide is 2-thiocytidine (s2C). For example, the chemically modified nucleotide is 2-thiouridine (s2U). For example, the chemically modified nucleotide is 5-methylcytidine (m5C). For example, the chemically modified nucleotide is N6-methyladenosine (m6a). For example, the chemically modified nucleotide is pseudouridine (ψ). For example, the chemically modified nucleotide is 1-methylpseudouridine (m1ψ).

In one example, the first nucleotide sequence comprises the 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 44.

In one example, the 5′-UTR, the fragment and/or the variant thereof is between 40 and 2000 nucleotides in length. For example, the 5′-UTR, the fragment and/or the variant thereof is between 40 and 100 nucleotides in length. For example, the 5′-UTR, the fragment and/or the variant thereof is between 100 and 250 nucleotides in length. For example, the 5′-UTR, the fragment and/or the variant thereof is between 250 and 500 nucleotides in length. For example, the 5′-UTR, the fragment and/or the variant thereof is between 500 and 750 nucleotides in length. For example, the 5′-UTR, the fragment and/or the variant thereof is between 750 and 1000 nucleotides in length. For example, the 5′-UTR, the fragment and/or the variant thereof is between 1000 and 1250 nucleotides in length. For example, the 5′-UTR, the fragment and/or the variant thereof is between 1250 and 1500 nucleotides in length. For example, the 5′-UTR, the fragment and/or the variant thereof is between 1500 and 1750 nucleotides in length. For example, the 5′-UTR, the fragment and/or the variant thereof is between 1750 and 2000 nucleotides in length.

In one example, the 5′-UTR, the fragment and/or the variant thereof comprises a nucleotide sequence at least 90% identical to a nucleotide sequence set forth in any one of SEQ ID NO: 40 to 44. For example, the 5′-UTR, the fragment and/or the variant thereof comprises a nucleotide sequence 90%, or 91%, or 92%, or 93%, or 94%, or 95%, or 96%, or 97%, or 98%, or 99% identical to a nucleotide sequence set forth in any one of SEQ ID NO: 40 to 44.

In one example, the polynucleotide comprises a combination of two or more 5′-UTRs, fragments and/or variants thereof. In one example, the two or more 5′-UTRs are the same. In one example, the two or more 5′-UTRs are different.

In one example, the nucleotide sequence comprising the 5′UTR comprises at least one microRNA binding site, an AU rich element (ARE), a GC-rich element, a stem loop, and combinations thereof. In one example, the nucleotide sequence comprises a microRNA binding site. In one example, the nucleotide sequence comprises an AU rich element (ARE). In one example, the nucleotide comprises a GC-rich element. In one example, the nucleotide sequence comprises a stem loop. For example, the stem loop is a histone stem loop.

In one example, the polynucleotide further comprises a nucleotide sequence comprising a 3′UTR. In one example, the nucleotide sequence comprising the 3′UTR is located 3′ of the second or the one or more additional nucleotide sequences. For example, the nucleotide sequence comprising the 3′UTR is located 3′ of the second nucleotide sequence. In one example, the 3′UTR comprises a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 45.

In one example, the 3′UTR, the fragment and/or the variant thereof is between 40 and 400 nucleotides in length. For example, the 3′-UTR is between 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300, or 300 and 325, or 325 and 350, or 350 and 375, or 375 and 400 nucleotides in length. For example, the 3′-UTR, the fragment and/or the variant thereof is between 40 and 50 nucleotides in length. For example, the 3′-UTR, the fragment and/or the variant thereof is between 50 and 60 nucleotides in length. For example, the 3′-UTR, the fragment and/or the variant thereof is between 60 and 70 nucleotides in length. For example, the 3′-UTR, the fragment and/or the variant thereof is between 70 and 80 nucleotides in length. For example, the 3′-UTR, the fragment and/or the variant thereof is between 80 and 90 nucleotides in length. For example, the 3′-UTR, the fragment and/or the variant thereof is between 90 and 100 nucleotides in length. For example, the 3′-UTR, the fragment and/or the variant thereof is between 100 and 125 nucleotides in length. For example, the 3′-UTR, the fragment and/or the variant thereof is between 125 and 150 nucleotides in length. For example, the 3′-UTR, the fragment and/or the variant thereof is between 150 and 175 nucleotides in length. For example, the 3′-UTR, the fragment and/or the variant thereof is between 175 and 200 nucleotides in length. For example, the 3′-UTR, the fragment and/or the variant thereof is between 200 and 225 nucleotides in length. For example, the 3′-UTR, the fragment and/or the variant thereof is between 225 and 250 nucleotides in length. For example, the 3′-UTR, the fragment and/or the variant thereof is between 250 and 275 nucleotides in length. For example, the 3′-UTR, the fragment and/or the variant thereof is between 275 and 300 nucleotides in length. For example, the 3′-UTR, the fragment and/or the variant thereof is between 300 and 325 nucleotides in length. For example, the 3′-UTR, the fragment and/or the variant thereof is between 325 and 350 nucleotides in length. For example, the 3′-UTR, the fragment and/or the variant thereof is between 350 and 375 nucleotides in length. For example, the 3′-UTR, the fragment and/or the variant thereof is between 375 and 400 nucleotides in length.

In one example, the polynucleotide comprises a combination of two or more 3′-UTRs, fragments and/or variants thereof. In one example, the two or more 3′-UTRs are the same. In one example, the two or more 3′-UTRs are different.

In one example, the nucleotide sequence comprising the 3′UTR, the fragment and/or variant thereof comprises at least one microRNA binding site, an AU rich element (ARE), a GC-rich element, a triple helix, a stem loop, one or more stop codons and combinations thereof. In one example, the nucleotide sequence comprises a microRNA binding site. In one example, the nucleotide sequence comprises an AU rich element (ARE). In one example, the nucleotide sequence comprises a GC-rich element. In one example, the nucleotide sequence comprises a triple helix. In one example, the nucleotide sequence comprises a stem loop. For example, the stem loop is a histone stem loop. In one example, the nucleotide sequence comprises one or more stop codons. For example, the one or more stop codons are located at the 5′end of the 3′-UTR.

In one example, the polynucleotide comprises a nucleotide sequence comprising one or more 3′ tailing sequences located at the 3′end of the nucleotide sequence comprising the 3′UTR. In one example, the one or more 3′ tailing sequences are selected from the group consisting of a poly-A sequence, polyadenylation signal, a G-quadruplex, a poly-C sequence, a stem loop and combinations thereof. For example, the 3′ tailing sequence comprises a poly-A sequence. In one example, the 3′ tailing sequence comprises a polyadenylation signal. In one example, the 3′ tailing sequence comprises a G-quadruplex. In one example, the 3′ tailing sequence comprises a poly-C sequence. In one example, the 3′ tailing sequence comprises a stem loop. For example, the stem loop is a histone stem loop. In one example, the 3′ tailing sequence comprises a poly-A sequence and a G-quadruplex. In one example, the 3′ tailing sequence comprises a stem loop (e.g., a histone stem loop) and a poly-A sequence.

In one example, the one or more 3′ tailing sequences comprises one or more poly-A sequences each comprising between 10 and 300 consecutive adenosine nucleotides. For example, the poly-A sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive adenosine nucleotides. For example, the one or more poly-A sequences each comprises between 10 and 20 consecutive adenosine nucleotides. For example, the one or more poly-A sequences each comprises between 20 and 30 consecutive adenosine nucleotides. For example, the one or more poly-A sequences each comprises between 30 and 40 consecutive adenosine nucleotides. For example, the one or more poly-A sequences each comprise 36 consecutive adenosine nucleotides. For example, the one or more poly-A sequences each comprises between 40 and 50 consecutive adenosine nucleotides. For example, the one or more poly-A sequences each comprises between 50 and 60 consecutive adenosine nucleotides. For example, the one or more poly-A sequences each comprises between 60 and 70 consecutive adenosine nucleotides. For example, the one or more poly-A sequences each comprises between 70 and 80 consecutive adenosine nucleotides. For example, the one or more poly-A sequences each comprises between 80 and 90 consecutive adenosine nucleotides. For example, the one or more poly-A sequences each comprises between 90 and 100 consecutive adenosine nucleotides. For example, the one or more poly-A sequences each comprises between 100 and 125 consecutive adenosine nucleotides. For example, the one or more poly-A sequences each comprises between 125 and 150 consecutive adenosine nucleotides. For example, the one or more poly-A sequences each comprises between 150 and 175 consecutive adenosine nucleotides. For example, the one or more poly-A sequences each comprises between 175 and 200 consecutive adenosine nucleotides. For example, the one or more poly-A sequences each comprises between 200 and 225 consecutive adenosine nucleotides. For example, the one or more poly-A sequences each comprises between 225 and 250 consecutive adenosine nucleotides. For example, the one or more poly-A sequences each comprises between 250 and 275 consecutive adenosine nucleotides. For example, the one or more poly-A sequences each comprises between 275 and 300 consecutive adenosine nucleotides.

In one example, the one or more poly-A sequence each comprises 10, or 20, or 30, or 40, or 50, or 60, or 70, or 80, or 90, or 100, or 125, or 150, or 175, or 200, or 225, or 250, or 275, or 300 consecutive adenosine nucleotides. For example, the one or more poly-A sequence each comprises 10 consecutive adenosine nucleotides. For example, the one or more poly-A sequence each comprises 20 consecutive adenosine nucleotides. For example, the one or more poly-A sequence each comprises 30 consecutive adenosine nucleotides. For example, the one or more poly-A sequence each comprises 40 consecutive adenosine nucleotides. For example, the one or more poly-A sequence each comprises 50 consecutive adenosine nucleotides. For example, the one or more poly-A sequence each comprises 60 consecutive adenosine nucleotides. For example, the one or more poly-A sequence each comprises 70 consecutive adenosine nucleotides. For example, the one or more poly-A sequence each comprises 80 consecutive adenosine nucleotides. For example, the one or more poly-A sequence each comprises 90 consecutive adenosine nucleotides. For example, the one or more poly-A sequence each comprises 100 consecutive adenosine nucleotides. For example, the one or more poly-A sequence each comprises 125 consecutive adenosine nucleotides. For example, the one or more poly-A sequence each comprises 150 consecutive adenosine nucleotides. For example, the one or more poly-A sequence each comprises 175 consecutive adenosine nucleotides. For example, the one or more poly-A sequence each comprises 200 consecutive adenosine nucleotides. For example, the one or more poly-A sequence each comprises 225 consecutive adenosine nucleotides. For example, the one or more poly-A sequence each comprises 250 consecutive adenosine nucleotides. For example, the one or more poly-A sequence each comprises 275 consecutive adenosine nucleotides. For example, the one or more poly-A sequence each comprises 300 consecutive adenosine nucleotides.

In one example, the poly-A sequence comprises 36 consecutive adenosine nucleotides. For example, the poly-A sequence comprises a sequence set forth in SEQ ID NO: 47.

In one example, the one or more poly-A sequences is separated by an interrupting linker. For example, the 3′tailing sequence comprises, in order of 5′ to 3′: a poly-A sequence comprising consecutive adenosine nucleotides, an interrupting linker, and a further poly-A sequence comprising consecutive adenosine nucleotides.

In one example, the interrupting linker is from 10 to 50, or 50 to 100, or 100 to 150 nucleotides in length. For example, the interrupting linker is from 10 to 50 nucleotides in length. For example, the interrupting linker is from 50 to 100 nucleotides in length. For example, the interrupting linker is from 100 to 150 nucleotides in length.

In one example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length. For example, the interrupting linker is 1 nucleotide in length. For example, the interrupting linker is 2 nucleotides in length. For example, the interrupting linker is 3 nucleotides in length. For example, the interrupting linker is 4 nucleotides in length. For example, the interrupting linker is 5 nucleotides in length. For example, the interrupting linker is 6 nucleotides in length. For example, the interrupting linker is 7 nucleotides in length. For example, the interrupting linker is 8 nucleotides in length. For example, the interrupting linker is 9 nucleotides in length. For example, the interrupting linker is 10 nucleotides in length. For example, the interrupting linker is 11 nucleotides in length. For example, the interrupting linker is 12 nucleotides in length. For example, the interrupting linker is 13 nucleotides in length. For example, the interrupting linker is 14 nucleotides in length. For example, the interrupting linker is 15 nucleotides in length. For example, the interrupting linker is 16 nucleotides in length. For example, the interrupting linker is 17 nucleotides in length. For example, the interrupting linker is 18 nucleotides in length. For example, the interrupting linker is 19 nucleotides in length. For example, the interrupting linker is 20 nucleotides in length. For example, the interrupting linker is 25 nucleotides in length. For example, the interrupting linker is 30 nucleotides in length. For example, the interrupting linker is 35 nucleotides in length. For example, the interrupting linker is 40 nucleotides in length. For example, the interrupting linker is 45 nucleotides in length. For example, the interrupting linker is 50 nucleotides in length. For example, the interrupting linker is 55 nucleotides in length. For example, the interrupting linker is 60 nucleotides in length. For example, the interrupting linker is 65 nucleotides in length. For example, the interrupting linker is 70 nucleotides in length. For example, the interrupting linker is 75 nucleotides in length. For example, the interrupting linker is 80 nucleotides in length. For example, the interrupting linker is 85 nucleotides in length. For example, the interrupting linker is 90 nucleotides in length. For example, the interrupting linker is 95 nucleotides in length. For example, the interrupting linker is 100 nucleotides in length. For example, the interrupting linker is 110 nucleotides in length. For example, the interrupting linker is 120 nucleotides in length. For example, the interrupting linker is 130 nucleotides in length. For example, the interrupting linker is 140 nucleotides in length. For example, the interrupting linker is 150 nucleotides in length.

In one example, the interrupting linker is 10 nucleotides in length. In one example, the interrupting linker comprises or consists of the nucleotide sequence set forth in SEQ ID NO: 39. For example, the interrupting linker comprises or consists of a nucleotide sequence GCAUAUGACU.

In one example, the 3′ tailing sequence comprises, in order of 5′ to 3′: a poly-A sequence comprising 30 consecutive adenosine nucleotides, an interrupting linker of 10 nucleotides, and a further poly-A sequence comprising 70 consecutive adenosine nucleotides.

In one example, the 3′ tailing sequence comprises, in order of 5′ to 3′: a poly-A sequence comprising 30 consecutive adenosine nucleotides, an interrupting linker comprising or consisting of the nucleotide sequence set forth in SEQ ID NO: 39, and a further poly-A sequence comprising 70 consecutive adenosine nucleotides.

In one example, the polynucleotide comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an antigen of interest;
d) a second nucleotide sequence encoding a chemoattractant, optionally selected from the group consisting of chemerin, CXCL9, CXCL10 and CXCL11, operably linked to a regulatory element selected from the group consisting of a SG promoter and an IRES,
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the polynucleotide comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding a chemoattractant, optionally selected from the group consisting of chemerin, CXCL9, CXCL10 and CXCL11;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding a chemoattractant, optionally selected from the group consisting of chemerin, CXCL9, CXCL10 and CXCL11;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an antigen of interest;
d) a second nucleotide sequence encoding a chemoattractant, optionally selected from the group consisting of chemerin, CXCL9, CXCL10 and CXCL11, operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the cRNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding an antigen of interest;
d) a second nucleotide sequence encoding a chemoattractant, optionally selected from the group consisting of chemerin, CXCL9, CXCL10 and CXCL11, operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the cRNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding a chemoattractant, optionally selected from the group consisting of chemerin, CXCL9, CXCL10 and CXCL11;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the self-replicating RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) the first nucleotide sequence encoding an antigen of interest;
d) the second nucleotide sequence encoding a chemoattractant, optionally selected from the group consisting of chemerin, CXCL9, CXCL10 and CXCL11, operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the self-replicating RNA comprises, in order from 5′ to 3′:
a) a 5′-UTR, fragment and/or variant thereof;
b) a regulatory element selected from the group consisting of a Kozak consensus sequence, an IRES, a SG promoter and combinations thereof;
c) a first nucleotide sequence encoding a chemoattractant, optionally selected from the group consisting of chemerin, CXCL9, CXCL10 and CXCL11;
d) a second nucleotide sequence encoding an antigen of interest operably linked to a regulatory element selected from the group consisting of a SG promoter and an IRES;
e) a 3′-UTR, fragment and/or variant thereof; and
f) one or more 3′ tailing sequences selected from the group consisting of a poly-A sequence, polyadenylation signal, a G-quadruplex, a poly-C sequence, a stem loop and combinations thereof.

In one example, the self-replicating RNA of the present disclosure comprises, in order from 5′ to 3′:
a) a first nucleotide sequence encoding an antigen of interest operably linked to a minimal SG promoter; and a second nucleotide sequence encoding a chemoattractant, optionally selected from the group consisting of chemerin, CXCL9, CXCL10 and CXCL11, operably linked to a minimal SG promoter; or
b) a first nucleotide sequence encoding an antigen of interest operably linked to a minimal SG promoter; and a second nucleotide sequence encoding a chemoattractant, optionally selected from the group consisting of chemerin, CXCL9, CXCL10 and CXCL11, operably linked to an extended SG promoter; or
c) a first nucleotide sequence encoding an antigen of interest operably linked to a minimal SG promoter; and a second nucleotide sequence encoding a chemoattractant, optionally selected from the group consisting of chemerin, CXCL9, CXCL10 and CXCL11, operably linked to an a wild-type EMCV IRES;

In one example, the self-replicating RNA of the present disclosure comprises, in order from 5′ to 3′:
a) a first nucleotide sequence encoding a chemoattractant, optionally selected from the group consisting of chemerin, CXCL9, CXCL10 and CXCL11, operably linked to a minimal SG promoter; and a second nucleotide sequence encoding an antigen of interest operably linked to a minimal SG promoter; or
b) a first nucleotide sequence encoding a chemoattractant, optionally selected from the group consisting of chemerin, CXCL9, CXCL10 and CXCL11, operably linked to a minimal SG promoter; and a second nucleotide sequence encoding an antigen of interest operably linked to an extended SG promoter; or
c) a first nucleotide sequence encoding a chemoattractant, optionally selected from the group consisting of chemerin, CXCL9, CXCL10 and CXCL11, operably linked to a minimal SG promoter, and a second nucleotide sequence encoding an antigen of interest operably linked to an a wild-type EMCV IRES.

In one example, the self-replicating RNA of the present disclosure comprises, in order from 5′ to 3′: a first nucleotide sequence encoding an antigen of interest operably linked to a minimal SG promoter; and a second nucleotide sequence encoding a chemoattractant, optionally selected from the group consisting of chemerin, CXCL9, CXCL10 and CXCL11, operably linked to a minimal SG promoter.

In one example, the self-replicating RNA of the present disclosure comprises, in order from 5′ to 3′: a first nucleotide sequence encoding a chemoattractant, optionally selected from the group consisting of chemerin, CXCL9, CXCL10 and CXCL11, operably linked to a minimal SG promoter; and a second nucleotide sequence encoding an antigen of interest operably linked to a minimal SG promoter.

For example, the self-replicating RNA of the present disclosure comprises, in order from 5′ to 3′: a first nucleotide sequence encoding an antigen of interest operably linked to a minimal SG promoter comprising a sequence set forth in SEQ ID NO: 32; and a second nucleotide sequence encoding a chemoattractant, optionally selected from the group consisting of chemerin, CXCL9, CXCL10 and CXCL11, operably linked to a minimal SG promoter comprising a sequence set forth in SEQ ID NO: 32.

For example, the self-replicating RNA of the present disclosure comprises, in order from 5′ to 3′: a first nucleotide sequence encoding a chemoattractant, optionally selected from the group consisting of chemerin, CXCL9, CXCL10 and CXCL11, operably linked to a minimal SG promoter comprising a sequence set forth in SEQ ID NO: 32; and a second nucleotide sequence encoding an antigen of interest operably linked to a minimal SG promoter comprising a sequence set forth in SEQ ID NO: 32.

In one example, the self-replicating RNA of the present disclosure comprises, in order from 5′ to 3′: a first nucleotide sequence encoding an antigen of interest operably linked to a minimal SG promoter; and a second nucleotide sequence encoding a chemoattractant, optionally selected from the group consisting of chemerin, CXCL9, CXCL10 and CXCL11, operably linked to an extended SG promoter.

In one example, the self-replicating RNA of the present disclosure comprises, in order from 5′ to 3′: a first nucleotide sequence encoding a chemoattractant, optionally selected from the group consisting of chemerin, CXCL9, CXCL10 and CXCL11, operably linked to a minimal SG promoter; and a second nucleotide sequence encoding an antigen of interest operably linked to an extended SG promoter.

In one example, the self-replicating RNA of the present disclosure comprises, in order from 5′ to 3′: a first nucleotide sequence encoding an antigen of interest operably linked to a minimal SG promoter encoded by a sequence set forth in SEQ ID NO: 32; and a second nucleotide sequence encoding a chemoattractant, optionally selected from the group consisting of chemerin, CXCL9, CXCL10 and CXCL11, operably linked to an extended SG promoter encoded by a sequence set forth in SEQ ID NO: 33.

In one example, the self-replicating RNA of the present disclosure comprises, in order from 5′ to 3′: a first nucleotide sequence encoding a chemoattractant, optionally selected from the group consisting of chemerin, CXCL9, CXCL10 and CXCL11, operably linked to a minimal SG promoter encoded by a sequence set forth in SEQ ID NO: 32; and a second nucleotide sequence encoding an antigen of interest operably linked to an extended SG promoter encoded by a sequence set forth in SEQ ID NO: 33.

In one example, the self-replicating RNA of the present disclosure comprises, in order from 5′ to 3′: a first nucleotide sequence encoding an antigen of interest operably linked to a minimal SG promoter; and a second nucleotide sequence encoding a chemoattractant, optionally selected from the group consisting of chemerin, CXCL9, CXCL10 and CXCL11, operably linked to a wild-type EMCV IRES.

In one example, the self-replicating RNA of the present disclosure comprises, in order from 5′ to 3′: a first nucleotide sequence encoding a chemoattractant, optionally selected from the group consisting of chemerin, CXCL9, CXCL10 and CXCL11, operably linked to a minimal SG promoter; and a second nucleotide sequence encoding an antigen of interest operably linked to a wild-type EMCV IRES.

In one example, the self-replicating RNA of the present disclosure comprises, in order from 5′ to 3′: a first nucleotide sequence encoding antigen of interest operably linked to a minimal SG promoter encoded by a sequence set forth in SEQ ID NO: 32 and a second nucleotide sequence encoding a chemoattractant, optionally selected from the group consisting of chemerin, CXCL9, CXCL10 and CXCL11, operably linked to a wild-type EMCV IRES encoded by a sequence set forth in SEQ ID NO: 35.

In one example, the self-replicating RNA of the present disclosure comprises, in order from 5′ to 3′: a first nucleotide sequence encoding a chemoattractant, optionally selected from the group consisting of chemerin, CXCL9, CXCL10 and CXCL11, operably linked to a minimal SG promoter encoded by a sequence set forth in SEQ ID NO: 32 and a second nucleotide sequence encoding an antigen of interest operably linked to a wild-type EMCV IRES encoded by a sequence set forth in SEQ ID NO: 35.

In one example, the RNA further comprises a 5′ terminal cap structure.

In one example, the 5′ terminal cap structure is an endogenous cap or analogue thereof. For example, the 5′terminal cap structure is an endogenous cap. For example, the 5′terminal cap structure is an analogue of an endogenous cap.

In one example, the 5′ terminal cap structure comprise a guanine or guanine analogue thereof. For example, the 5′ terminal cap structure comprise a guanine. For example, the 5′ terminal cap structure comprise a guanine analogue of a guanine.

In one example, the 5′ terminal cap structure is selected from a group consisting of anti-reverse cap analogue (ARCA), N7,2′-0-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2. For example, the 5′ terminal cap structure is anti-reverse cap analogue (ARCA). For example, the 5′ terminal cap structure is N7,2′-0-dimethyl-guanosine (mCAP). For example, the 5′ terminal cap structure is inosine. For example, the 5′ terminal cap structure is N1-methyl-guanosine. For example, the 5′ terminal cap structure is 2′fluoro-guanosine. For example, the 5′ terminal cap structure is 7-deaza-guanosine. For example, the 5′ terminal cap structure is 8-oxo-guanosine. For example, the 5′ terminal cap structure is 2-amino-guanosine. For example, the 5′ terminal cap structure is LNA-guanosine. For example, the 5′ terminal cap structure is 2-azido-guanosine. For example, the 5′ terminal cap structure is N6,2′-O-dimethyladenosine. For example, the 5′ terminal cap structure is 7-methylguanosine (m7G). For example, the 5′ terminal cap structure is Cap1. For example, the 5′ terminal cap structure is Cap2.

In one example, the 5′terminal cap structure is linked to the 5′ end of the RNA by a 5′-5′-triphosphate linkage or a 5′-5′ phosphorothioate linkage. For example, the 5′terminal cap structure is linked to the 5′ end of the RNA by a 5′-5′-triphosphate linkage. For example, the 5′terminal cap structure is linked to the 5′ end of the RNA by a 5′-5′ phosphorothioate linkage.

In one example, the self-replicating RNA is from an alphavirus. For example, the alphavirus is selected from the group consisting of Semliki Forest virus (SFV), Sindbis virus (SIN), and Venezuelan equine encephalitis virus (VEEV) and combinations thereof.

In one example, the self-replicating RNA is from a Semliki Forest virus (SFV).

In one example, the self-replicating RNA is from a Sindbis virus (SIN).

In one example, the self-replicating RNA is from a Venezuelan equine encephalitis virus (VEEV).

In one example, the antigen is a viral antigen. For example, the viral antigen is from a respiratory virus. In one example, the respiratory virus is selected from the group consisting of influenza virus, respiratory syncytial virus, parainfluenza viruses, metapneumovirus, rhinovirus, coronaviruses, adenoviruses and bocaviruses. In another example, the respiratory virus is selected from the group consisting of bronchiolitis, pneumonia, croup, a SARS-CoV-2 infection, coronavirus disease 2019 (COVID-19), acute respiratory disease syndrome (ARDS) and combinations thereof.

In one example, the viral antigen is from an influenza virus.

In one example, the viral antigen is from a respiratory syncytial virus.

In one example, the viral antigen is from a parainfluenza virus.

In one example, the viral antigen is from a metapneumovirus.

In one example, the viral antigen is from a rhinovirus.

In one example, the viral antigen is from a coronavirus.

In one example, the viral antigen is from an adenovirus.

In one example, the viral antigen is from a bocavirus.

In one example, the viral antigen is from bronchiolitis.

In one example, the viral antigen is from pneumonia.

In one example, the viral antigen is from croup.

In one example, the viral antigen is from a SARS-CoV-2 infection.

In one example, the viral antigen is from coronavirus disease 2019 (COVID-19).

In one example, the viral antigen is from acute respiratory disease syndrome (ARDS).

In one example, the antigen is a viral antigen from an influenza virus or a coronavirus.

In one example, the self-replicating RNA is from a VEEV and the antigen is a H5 antigen from influenza. For example, a self-replicating RNA suitable for use in combination with a chemoattractant polypeptide is set forth in SEQ ID NO: 48 or 49. In this example, the self-replicating RNA set forth in SEQ ID NO:48 or 49 may be used in combination with a chemoattractant polypeptide having an amino acid sequence set forth in any one of SEQ ID NOs: 3, 6, 9, 12, 15, 18 or 21. In another example, the present disclosure contemplates use of a self-replicating RNA having at least about 70%, at least about 75%, at least about 80%, at least about 85%, at least about 90% at least about 95%, at least about 96%, at least about 97%, at least about 98%, at least about 99% or 100% sequence identity to SEQ ID NO: 48 or 49, and a chemoattractant polypeptide having an amino acid sequence having at least about 70%, at least about 75%, at least about 80%, at least about 85%, at least about 90% at least about 95%, at least about 96%, at least about 97%, at least about 98%, at least about 99% or 100% sequence identity to any one of SEQ ID NOs: 3, 6, 9, 12, 15, 18 or 21.

In one example, the nucleotide sequence encoding a chemoattractant is selected from the group consisting of chemerin, C-X-C motif chemokine ligand 9 (CXCL9), C-X-C motif chemokine ligand 10 (CXCL10) and C-X-C motif chemokine ligand 11 (CXCL11).

In an example, the nucleotide sequence encoding a chemoattractant is chemerin.

In an example, the nucleotide sequence encoding a chemoattractant is CXCL9.

In an example, the nucleotide sequence encoding a chemoattractant is CXCL10.

In an example, the nucleotide sequence encoding a chemoattractant is CXCL11.

In an example, the mRNA sequence encoding a chemoattractant comprises a polynucleotide sequence having at least about 70%, at least about 75%, at least about 80%, at least about 85%, at least about 90% at least about 95%, at least about 96%, at least about 97%, at least about 98%, at least about 99% or 100% sequence identity to any one of any one of SEQ ID NOs: 1, 4, 7, 10, 13, 16, 19, 22, 25 or 28.

In an example, the DNA sequence encoding a chemoattractant comprises a polynucleotide sequence having at least about 70%, at least about 75%, at least about 80%, at least about 85%, at least about 90% at least about 95%, at least about 96%, at least about 97%, at least about 98%, at least about 99% or 100% sequence identity to any one of any one of SEQ ID NOs: 2, 5, 8, 11, 14, 17, 20, 23, 26 or 29.

The present disclosure provides an immunogenic composition comprising the polynucleotide of the present disclosure. The present disclosure further provides an immunogenic composition comprising the RNA of the present disclosure. For example, the present disclosure provides an immunogenic composition comprising the cRNA of the present disclosure. The present disclosure also provides an immunogenic composition comprising the self-replicating RNA of the present disclosure. For example, the composition of the present disclosure, when administered, is capable of inducing an immune response in the subject. For example, administration of the composition induces a humoral and/or a cell-mediated immune response. In one example, the composition induces a humoral immune response in the subject. For example, the humoral immune response is an antibody-mediated immune response. In another example, the composition induces a cell-mediated immune response.

For example, the cell-mediated immune response includes activation of antigen-specific cytotoxic T cells.

In one example, an immunogenic composition of the disclosure comprises multiple polynucleotides, wherein each polynucleotide encodes different polypeptide sequences. In another example, an immunogenic composition of the disclosure comprises multiple RNAs, wherein each RNA encodes different polypeptide sequences. In a further example, an immunogenic composition of the disclosure comprises multiple cRNAs, wherein each cRNA encodes different polypeptide sequences. In one example, an immunogenic composition comprises multiple multicistronic self-replicating RNAs, wherein each multicistronic self-replicating RNA encodes a polypeptide of an antigen of interest and a chemoattractant described herein. In another example, an immunogenic composition of the disclosure comprises a plurality of self-replicating monocistronic RNAs, wherein each self-replicating RNA encodes different polypeptide sequences. For example, one self-replicating RNA encodes a polypeptide of an antigen of interest and a different self-replicating RNA encodes a chemoattractant described herein.

The present disclosure also provides an immunogenic composition comprising a polynucleotide of the present disclosure and a chemoattractant polypeptide. In an example, the chemoattractant polypeptide is selected from the group consisting of chemerin, C-X-C motif chemokine ligand 9 (CXCL9), C-X-C motif chemokine ligand 10 (CXCL10) and C-X-C motif chemokine ligand 11 (CXCL11).

For example, the immunogenic composition comprises:
(i) a polynucleotide comprising a first nucleotide sequence encoding an antigen operably linked to a regulatory element; and
(ii) a chemoattractant polypeptide.

In another example, the immunogenic composition comprises:
(i) a RNA comprising a first nucleotide sequence encoding an antigen operably linked to a regulatory element; and
(ii) a chemoattractant polypeptide.

In another example, the immunogenic composition comprises:
(i) a RNA comprising a first nucleotide sequence encoding an antigen operably linked to a regulatory element; and
(ii) a chemoattractant polypeptide selected from the group consisting of chemerin, C-X-C motif chemokine ligand 9 (CXCL9), C-X-C motif chemokine ligand 10 (CXCL10) and C-X-C motif chemokine ligand 11 (CXCL11).

In another example, the immunogenic composition comprises:
(i) a cRNA comprising a first nucleotide sequence encoding an antigen operably linked to a regulatory element; and
(ii) a chemoattractant polypeptide.

In another example, the immunogenic composition comprises:
(i) a self-replicating RNA comprising a first nucleotide sequence encoding an antigen operably linked to a regulatory element; and
(ii) a chemoattractant polypeptide selected from the group consisting of chemerin, C-X-C motif chemokine ligand 9 (CXCL9), C-X-C motif chemokine ligand 10 (CXCL10) and C-X-C motif chemokine ligand 11 (CXCL11).

In an example, the regulatory element is selected from the group consisting of a promoter, optionally a subgenomic (SG) promoter, an internal ribosome entry site (IRES) and a Kozak consensus sequence or a combination thereof. In an example, the regulatory element is a SG promoter.

For example, the polynucleotide of the immunogenic composition comprises a first nucleotide sequence encoding an antigen operably linked to a regulatory element selected from a SG promoter, and IRES and/or a Kozak consensus sequence.

In another example, the RNA of the immunogenic composition comprises a first nucleotide sequence encoding an antigen operably linked to a regulatory element selected from a SG promoter, and IRES and/or a Kozak consensus sequence.

In another example, the cRNA of the immunogenic composition comprises a first nucleotide sequence encoding an antigen operably linked to a regulatory element selected from a SG promoter, and IRES and/or a Kozak consensus sequence.

In another example, the self-replicating RNA of the immunogenic composition comprises a first nucleotide sequence encoding an antigen operably linked to a regulatory element selected from a SG promoter, and IRES and/or a Kozak consensus sequence.

In one example, the immunogenic composition comprises:
(i) a polynucleotide comprising a first nucleotide sequence encoding an antigen operably linked to a regulatory element, and a second nucleotide sequence encoding a chemoattractant, optionally selected from the group consisting of chemerin, CXCL9, CXCL10 and CXCL11, operably linked to a regulatory element; and
(ii) a chemoattractant polypeptide, optionally selected from the group consisting of chemerin, CXCL9, CXCL10 and CXCL11.

In another example, the immunogenic composition comprises:
(i) a RNA comprising a first nucleotide sequence encoding an antigen operably linked to a regulatory element, and a second nucleotide sequence encoding a chemoattractant, optionally selected from the group consisting of chemerin, CXCL9, CXCL10 and CXCL11, operably linked to a regulatory element; and
(ii) a chemoattractant polypeptide, optionally selected from the group consisting of chemerin, CXCL9, CXCL10 and CXCL11.

In another example, the immunogenic composition comprises:
(i) a cRNA comprising a first nucleotide sequence encoding an antigen operably linked to a regulatory element, and a second nucleotide sequence encoding a chemoattractant, optionally selected from the group consisting of chemerin, CXCL9, CXCL10 and CXCL11, operably linked to a regulatory element; and
(ii) a chemoattractant polypeptide, optionally selected from the group consisting of chemerin, CXCL9, CXCL10 and CXCL11.

In another example, the immunogenic composition comprises:
(i) a self-replicating RNA comprising a first nucleotide sequence encoding an antigen operably linked to a regulatory element, and a second nucleotide sequence encoding a chemoattractant, optionally selected from the group consisting of chemerin, CXCL9, CXCL10 and CXCL11, operably linked to a regulatory element; and
(ii) a chemoattractant polypeptide, optionally selected from the group consisting of chemerin, CXCL9, CXCL10 and CXCL11.

Accordingly, the present disclosure provides an immunogenic composition comprising:
(i) an RNA comprising: a) a first nucleotide sequence encoding a first antigen of interest; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11; and b) a second nucleotide sequence encoding an antigen of interest operably linked to a regulatory element selected from the group consisting of a SG promoter and an IRES.

The present disclosure further provides an immunogenic composition comprising:
(i) a self-replicating RNA comprising: a) a first nucleotide sequence encoding a first antigen of interest; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11; and b) a second nucleotide sequence encoding an antigen of interest operably linked to a regulatory element selected from the group consisting of a SG promoter and an IRES.

The present disclosure further provides an immunogenic composition comprising
(i) a RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

The present disclosure provides an immunogenic composition comprising
(i) a self-replicating RNA comprising: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest operably linked to a regulatory element selected from the group consisting of a Kozak consensus sequence, an IRES, a SG promoter and combinations thereof; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a regulatory element selected from the group consisting of a Kozak consensus sequence, an IRES, a SG promoter and combinations thereof; and b) a second nucleotide sequence encoding an antigen of interest operably linked to a regulatory element selected from the group consisting of a SG promoter and an IRES.

The present disclosure provides an immunogenic composition comprising:
(i) a RNA comprising: a) a first nucleotide sequence encoding a first antigen of interest operably linked to a Kozak consensus sequence; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES, and
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest operably linked to a Kozak consensus sequence; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence; and b) a second nucleotide sequence encoding an antigen of interest operably linked to a regulatory element selected from the group consisting of a SG promoter and an IRES.

The present disclosure provides an immunogenic composition comprising:
(i) a RNA comprising: a) a first nucleotide sequence encoding a first antigen of interest operably linked to a Kozak consensus sequence and a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest operably linked to a Kozak consensus sequence and a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and a SG promoter; and b) a second nucleotide sequence encoding an antigen of interest operably linked to a regulatory element selected from the group consisting of a SG promoter and an IRES.

The present disclosure provides an immunogenic composition comprising:
(i) a RNA comprising: a) a first nucleotide sequence encoding a first antigen of interest operably linked to a Kozak consensus sequence and an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest operably linked to a Kozak consensus sequence and an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and an IRES; and b) a second nucleotide sequence encoding an antigen of interest operably linked to a regulatory element selected from the group consisting of a SG promoter and an IRES.

The present disclosure provides an immunogenic composition comprising:
(i) a RNA comprising: a) a first nucleotide sequence encoding a first antigen of interest operably linked to a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest operably linked to a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to a SG promoter; and b) a second nucleotide sequence encoding an antigen of interest operably linked to a regulatory element selected from the group consisting of a SG promoter and an IRES

The present disclosure provides an immunogenic composition comprising:
(i) a RNA comprising: a) a first nucleotide sequence encoding a first antigen of interest operably linked to an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES;
(ii) a chemoattractant polypeptide, optionally selected from the group consisting of chemerin, CXCL9, CXCL10 and CXCL11.

In one example, the RNA comprises, in order from 5′ to 3′: a) a first nucleotide sequence encoding a first antigen of interest operably linked to an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the RNA comprises, in order from 5′ to 3′: a) a first nucleotide sequence encoding a chemoattractant, optionally selected from the group consisting of chemerin, CXCL9, CXCL10 and CXCL11, operably linked to an IRES; and b) a second nucleotide sequence encoding an antigen of interest operably linked to a regulatory element selected from the group consisting of a SG promoter and an IRES.

The present disclosure provides an immunogenic composition comprising:
(i) a self-replicating RNA comprising: a) a first nucleotide sequence encoding a first antigen of interest operably linked to a Kozak consensus sequence; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest operably linked to a Kozak consensus sequence; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence; and b) a second nucleotide sequence encoding an antigen of interest operably linked to a regulatory element selected from the group consisting of a SG promoter and an IRES.

The present disclosure provides an immunogenic composition comprising:
(i) a self-replicating RNA comprising: a) a first nucleotide sequence encoding a first antigen of interest operably linked to a Kozak consensus sequence and a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest operably linked to a Kozak consensus sequence and a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and a SG promoter; and b) a second nucleotide sequence encoding an antigen of interest operably linked to a regulatory element selected from the group consisting of a SG promoter and an IRES.

The present disclosure provides an immunogenic composition comprising:
(i) a self-replicating RNA comprising: a) a first nucleotide sequence encoding a first antigen of interest operably linked to a Kozak consensus sequence and an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest operably linked to a Kozak consensus sequence and an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a Kozak consensus sequence and an IRES; and b) a second nucleotide sequence encoding an antigen of interest operably linked to a regulatory element selected from the group consisting of a SG promoter and an IRES.

The present disclosure provides an immunogenic composition comprising:
(i) a self-replicating RNA comprising: a) a first nucleotide sequence encoding a first antigen of interest operably linked to a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES; and
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest operably linked to a SG promoter; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to a SG promoter; and b) a second nucleotide sequence encoding an antigen of interest operably linked to a regulatory element selected from the group consisting of a SG promoter and an IRES.

The present disclosure provides an immunogenic composition comprising:
(i) a self-replicating RNA comprising: a) a first nucleotide sequence encoding a first antigen of interest operably linked to an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES;
(ii) a chemoattractant polypeptide, optionally selected from the group consisting of chemerin, CXCL9, CXCL10 and CXCL11.

In one example, the self-replicating RNA comprises, in order from 5′ to 3′: a) a first nucleotide sequence encoding a first antigen of interest operably linked to an IRES; and b) a second nucleotide sequence encoding a chemoattractant, optionally selected from the group consisting of chemerin, CXCL9, CXCL10 and CXCL11, operably linked to a regulatory element selected from the group consisting of a SG promoter and an IRES.

In one example, the self-replicating RNA comprises, in order from 5′ to 3′: a) a first nucleotide sequence encoding a chemoattractant, optionally selected from the group consisting of chemerin, CXCL9, CXCL10 and CXCL11, operably linked to an IRES; and b) a second nucleotide sequence encoding an antigen of interest operably linked to a regulatory element selected from the group consisting of a SG promoter and an IRES.

In one example, the chemoattractant polypeptide is selected from the group consisting of chemerin, C-X-C motif chemokine ligand 9 (CXCL9), C-X-C motif chemokine ligand 10 (CXCL10) and C-X-C motif chemokine ligand 11 (CXCL11).

In an example, the chemoattractant polypeptide is chemerin.

In an example, the chemoattractant polypeptide is CXCL9.

In an example, the chemoattractant polypeptide is CXCL10.

In an example, the chemoattractant polypeptide is CXCL11.

In an example, chemoattractant polypeptide comprises an amino acid sequence having at least about 70%, at least about 75%, at least about 80%, at least about 85%, at least about 90% at least about 95%, at least about 96%, at least about 97%, at least about 98%, at least about 99% or 100% sequence identity to any one of SEQ ID NOs: 3, 6, 9, 12, 15, 18 or 21.

The present disclosure also provides a pharmaceutical composition comprising an immunogenic composition of the present disclosure and a pharmaceutically acceptable carrier. Pharmaceutically acceptable carriers suitable for use in the present disclosure will be apparent to the skilled person and/or are described herein.

In one example, the pharmaceutical composition further comprises a lipid nanoparticle (LNP), a polymeric microparticle, and an oil-in-water emulsion. For example, the polynucleotide, the RNA, the cRNA or the self-replicating RNA is encapsulated in, bound to or adsorbed on a LNP, a polymeric microparticle, and an oil-in-water emulsion. In one example, the polynucleotide is encapsulated in, bound to or adsorbed on a LNP, a polymeric microparticle, and an oil-in-water emulsion. In another example, the RNA is encapsulated in, bound to or adsorbed on a LNP, a polymeric microparticle, and an oil-in-water emulsion. For example, the cRNA is encapsulated in, bound to or adsorbed on a LNP, a polymeric microparticle, and an oil-in-water emulsion. For example, the self-replicating RNA is encapsulated in, bound to or adsorbed on a LNP, a polymeric microparticle, and an oil-in-water emulsion.

In one example, the pharmaceutical composition further comprises a LNP. For example, the polynucleotide is encapsulated in a LNP. In another example, the RNA is encapsulated in a LNP. For example, the cRNA is encapsulated in a LNP. For example, the self-replicating RNA is encapsulated in a LNP. For example, the polynucleotide is bound to a LNP. In another example, the RNA is bound to a LNP. For example, the cRNA is bound to a LNP. In another example, the self-replicating RNA is bound to a LNP. For example, the polynucleotide is adsorbed on to a LNP. In another example, the RNA is adsorbed on to a LNP. For example, the cRNA is adsorbed on to a LNP. In a further example, the self-replicating RNA is adsorbed on to a LNP. In another example, wherein each RNA is formulated together in the LNP. In another example, wherein each RNA is formulated separately in the LNP.

In one example, the RNA encoding the chemoattractant and the RNA encoding the antigen are contained within the same LNP.

In one example, the composition further comprises an additional RNA encoding:
(i) one or more antigens;
(ii) one or more immunopotentiating agents;
(iii) one or more chemoattractants; and/or
(iv) one or more targeting molecules.

In one example, the additional RNA is contained within the same LNP as the RNA encoding the chemoattractant and/or the RNA encoding the antigen or is contained within a LNP separate to the RNA encoding the chemoattractant and the RNA encoding the antigen.

In one example, the LNP comprises a PEG-lipid, a structural lipid and/or a neutral lipid. For example, the LNP comprises a PEG-lipid, a structural lipid and a neutral lipid. In another example, the LNP comprises a PEG-lipid, a structural lipid or a neutral lipid.

In one example, the LNP further comprises a cationic lipid. In another example, the LNP does not comprise a cationic lipid.

In one example, the pharmaceutical composition further comprises a polymeric microparticle. For example, the polynucleotide is encapsulated in a polymeric microparticle. In another example, the RNA is encapsulated in a polymeric microparticle. For example, the cRNA is encapsulated in a polymeric microparticle. For example, the self-replicating RNA is encapsulated in a polymeric microparticle. For example, the polynucleotide is bound to a polymeric microparticle. In another example, the RNA is bound to a polymeric microparticle. For example, the cRNA is bound to a polymeric microparticle. In another example, the self-replicating RNA is bound to a polymeric microparticle.

For example, the polynucleotide is adsorbed on to a polymeric microparticle. In another example, the RNA is adsorbed on to a polymeric microparticle. For example, the cRNA is adsorbed on to a polymeric microparticle. In a further example, the self-replicating RNA is adsorbed on to a polymeric microparticle.

In one example, the pharmaceutical composition further comprises an oil-in-water emulsion. For example, the polynucleotide is encapsulated in an oil-in-water emulsion. In another example, the RNA is encapsulated in an oil-in-water emulsion. For example, the cRNA is encapsulated in an oil-in-water emulsion.

For example, the self-replicating RNA is encapsulated in an oil-in-water emulsion. For example, the polynucleotide is bound to an oil-in-water emulsion.

In another example, the RNA is bound to an oil-in-water emulsion. For example, the cRNA is bound to an oil-in-water emulsion. In another example, the self-replicating RNA is bound to an oil-in-water emulsion. In a further example, the self-replicating RNA is adsorbed on to an oil-in-water emulsion. In a further example, the self-replicating RNA is resuspended in an oil-in-water emulsion.

The present disclosure also provides an immunogenic composition or a pharmaceutical composition of the disclosure for use as a vaccine.

In one example, the polynucleotide is DNA. In one example, the disclosure provides a DNA encoding a cRNA vaccine of the disclosure. In one example, the disclosure provides a DNA encoding a self-replicating RNA vaccine of the disclosure.

In one example, the DNA is a plasmid.

The present disclosure provides a method of treating or preventing or delaying progression of a disease or condition in a subject, the method comprising administering the immunogenic composition or the pharmaceutical composition of the present disclosure to a subject in need thereof. In one example, the disclosure provides a method of treating a disease or condition in a subject, the method comprising administering the immunogenic composition or the pharmaceutical composition of the present disclosure to a subject in need thereof. In another example, the disclosure provides a method of preventing a disease or condition in a subject, the method comprising administering the immunogenic composition or the pharmaceutical composition of the present disclosure to a subject in need thereof. In a further example, the disclosure provides a method of delaying progression of a disease or condition in a subject, the method comprising administering the immunogenic composition or the pharmaceutical composition of the present disclosure to a subject in need thereof.

In one example, the present disclosure provides use of a polynucleotide of the disclosure in the manufacture of a medicament for treating or preventing or delaying progression of a disease or condition in a subject in need thereof. For example, the disclosure provides use of a polynucleotide of the disclosure in the manufacture of a medicament for treating a disease or condition in a subject in need thereof. In another example, the disclosure provides use of a polynucleotide of the disclosure in the manufacture of a medicament for preventing a disease or condition in a subject in need thereof. In a further example, the disclosure provides use of a polynucleotide of the disclosure in the manufacture of a medicament for delaying progression of a disease or condition in a subject in need thereof.

In one example, the present disclosure provides use of a RNA of the disclosure in the manufacture of a medicament for treating or preventing or delaying progression of a disease or condition in a subject in need thereof. For example, the disclosure provides use of a RNA of the disclosure in the manufacture of a medicament for treating a disease or condition in a subject in need thereof. In another example, the disclosure provides use of a RNA of the disclosure in the manufacture of a medicament for preventing a disease or condition in a subject in need thereof. In a further example, the disclosure provides use of a RNA of the disclosure in the manufacture of a medicament for delaying progression of a disease or condition in a subject in need thereof.

In one example, the present disclosure provides use of a cRNA of the disclosure in the manufacture of a medicament for treating or preventing or delaying progression of a disease or condition in a subject in need thereof. For example, the disclosure provides use of a cRNA of the disclosure in the manufacture of a medicament for treating a disease or condition in a subject in need thereof. In another example, the disclosure provides use of a cRNA of the disclosure in the manufacture of a medicament for preventing a disease or condition in a subject in need thereof. In a further example, the disclosure provides use of a cRNA of the disclosure in the manufacture of a medicament for delaying progression of a disease or condition in a subject in need thereof.

In one example, the present disclosure provides use of a self-replicating RNA of the disclosure in the manufacture of a medicament for treating or preventing or delaying progression of a disease or condition in a subject in need thereof. For example, the disclosure provides use of a self-replicating RNA of the disclosure in the manufacture of a medicament for treating a disease or condition in a subject in need thereof. In another example, the disclosure provides use of a self-replicating RNA of the disclosure in the manufacture of a medicament for preventing a disease or condition in a subject in need thereof. In a further example, the disclosure provides use of a self-replicating RNA of the disclosure in the manufacture of a medicament for delaying progression of a disease or condition in a subject in need thereof.

In one example, the present disclosure provides a self-replicating RNA of the disclosure for use in treating or preventing or delaying progression of a disease or condition in a subject in need thereof. For example, the disclosure provides a self-replicating RNA of the disclosure for use in treating a disease or condition in a subject in need thereof. In another example, the disclosure provides a self-replicating RNA of the disclosure for use in preventing a disease or condition in a subject in need thereof. In a further example, the disclosure provides a self-replicating RNA of the disclosure for use in delaying progression of a disease or condition in a subject in need thereof.

In one example, the subject suffers from a disease or condition. In one example, the subject has been diagnosed as suffering from a disease or condition. In one example, the subject is receiving treatment for a disease or condition.

In one example, the disease or condition is selected from the group consisting of influenza, COVID-19, respiratory syncytial virus (RSV) and acute respiratory distress syndrome (ARDS).

In an example, the present disclosure provides a method of inducing an immune response in a subject, the method comprising administering a RNA disclosed herein (e.g., self-replicating RNA), a pharmaceutical composition disclosed herein, an immunogenic composition disclosed herein or a vaccine disclosed herein to a subject in need thereof.

In an example, the present disclosure provides use of a RNA disclosed herein (e.g., self-replicating RNA), a pharmaceutical composition disclosed herein, an immunogenic composition disclosed herein or a vaccine disclosed herein in the manufacture of a medicament for inducing an immune response in a subject in need thereof.

In an example, the present disclosure provides a RNA disclosed herein (e.g., self-replicating RNA), a pharmaceutical composition disclosed herein, an immunogenic composition disclosed herein or a vaccine disclosed herein for use in inducing an immune response in a subject in need thereof.

In one example, the composition induces a humoral immune response in the subject. For example, the humoral immune response is an antibody-mediated immune response. For example, production of neutralizing antibodies. In another example, the composition induces a cell-mediated immune response. For example, the cell-mediated immune response includes activation of antigen-specific cytotoxic T cells. For example, the T cells are CD4 T cells and/or CD8 T cells. In one example, the T cells are CD4 T cells. In another example the T cells are CD8 T cells. In a further example, the T cells are CD4 and CD8 T cells.

In one example, administration of a RNA disclosed herein (e.g., self-replicating RNA), a pharmaceutical composition disclosed herein, an immunogenic composition disclosed herein or a vaccine of the present disclosure induces a CD4 T cell mediated immune response.

In one example, administration of a RNA disclosed herein (e.g., self-replicating RNA), a pharmaceutical composition, an immunogenic composition disclosed herein or a vaccine of the present disclosure induces a CD8 T cell mediated immune response.

In one example, administration of a RNA disclosed herein (e.g., self-replicating RNA), a pharmaceutical composition disclosed herein, an immunogenic composition disclosed herein or a vaccine of the present disclosure induces a CD4 and a CD8 T cell mediated immune response.

In one example, the CD4 T cell mediated immune response is a Th0, a Th1 and/or a Th2 response. For example, the CD4 T cell mediated immune response is a Th0 response. In another example, the CD4 T cell mediated immune response is a Th1 response. In a further example, the CD4 T cell mediated immune response is a Th2 response. In one example, the CD4 T cell mediated immune response is a Th0 and Th1 response. In another example, the CD4 T cell mediated immune response is a Th0 and Th2 response. In a further example, the CD4 T cell mediated immune response is a Th1 and Th2 response. In another example, the CD4 T cell mediated immune response is a Th0, Th1 and Th2 response.

In one example, the Th0 response cytokines express interleukin 2 (IL2+) and/or tumor necrosis factor alpha (TNFa+); and/or are negative for interferon gamma (IFNg−), IL5− and/or IL13−. For example, the cytokine is IL2+. In another example, the cytokine is TNFa+. In one example, the cytokine is IFNg−. In another example, the cytokine is IL5−. In a further example, the cytokine is IL13−

In one example, the Th1 response cytokines express interferon gamma (IFNg+); and/or are negative for IL5− and/or IL13−. For example, the cytokine is IFNg+. In another example, the cytokine is IL5−. In a further example, the cytokine is IL13−.

In one example, the Th2 response cytokines express IL5+ and/or IL13+; and/or are negative for IFNg. For example, the cytokine is IL5+. In a further example, the cytokine is IL13+. For example, the cytokine is IFNg−.

In an example, the present disclosure provides a method for reducing viral load in a subject having a viral infection comprising administering a RNA disclosed herein (e.g., self-replicating RNA), a pharmaceutical composition disclosed herein, an immunogenic composition disclosed herein or a vaccine disclosed herein to a subject having a viral infection.

In an example, the present disclosure provides use of a RNA disclosed herein (e.g., self-replicating RNA), a pharmaceutical composition disclosed herein, an immunogenic composition disclosed herein or a vaccine disclosed herein in the preparation of a medicament for reducing viral load in a subject having a viral infection.

In an example, the present disclosure provides a RNA disclosed herein (e.g., self-replicating RNA), a pharmaceutical composition disclosed herein, an immunogenic composition disclosed herein or a vaccine disclosed herein for use in reducing viral load in a subject having a viral infection.

In an example, the subject is a human of 18 years of age or older. In another example, the subject is a human of any age, e.g., from about 1 month to 100 years old, e.g., from about 2 months to about 80 years old, from about 6 months of age to about 3 years old, from about 3 years to about 18 years old, from about 12 years to about 18 years old, from about 18 years to about 55 years old, from about 50 years to about 75 years old, from about 40 years to about 65 years old. In another example, the subject is a human from 2 years of age. In another example, subject is a human from 18 years of age, a human from 30 years of age, a human from 40 years of age, a human from 50 years of age, a human from 60 years of age, a human from 70 years of age, a human from 80 years of age or a human from about 90 years of age. In another example, the subject is less than 2 years of age, less than 18 months of age, less than 12 months of age, less than 6 months of age or less than 3 months of age.

In an example, a composition or vaccine described herein is administered in a one dose regimen. In another example, the composition is administered in a two, three or four dose regimen, wherein the doses are administered about 1, 2 or 3 months apart.

The present disclosure also provides a polynucleotide that encodes the self-replicating RNA of the present disclosure. For example, the polynucleotide is a recombinant DNA. In one example, the recombinant DNA is a plasmid. In one example, the plasmid comprises a sequence set forth in SEQ ID NO: 50.

The present disclosure also provides a kit comprising at least one self-replicating RNA of the disclosure, optionally in a delivery system and/or a pharmaceutically acceptable carrier or diluent, packaged with instructions for use in treating or preventing or delaying progression of a disease or disorder in a subject.

The present disclosure also provides a kit comprising at least one self-replicating RNA of the disclosure, optionally in a delivery system and/or a pharmaceutically acceptable carrier or diluent, packaged with instructions to administer the RNA to a subject who is suffering from, or at risk of suffering from, a disease or disorder.

In one example, the self-replicating RNA, the RNA, the immunogenic composition or the pharmaceutical composition of the disclosure is supplied in a vial. In another example, the RNA, the self-replicating RNA, the immunogenic composition or the pharmaceutical composition of the disclosure is supplied in a syringe.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

KEY TO SEQUENCE LISTING

| | SEQ ID |
|---|---|
| Sequence | NO |
| mRNA sequence of Homo sapiens CXCL9 | 1 |
| DNA sequence of Homo sapiens CXCL9 | 2 |
| Amino acid sequence of Homo sapiens CXCL9 | 3 |
| mRNA sequence of Homo sapiens CXCL10 | 4 |
| DNA sequence of Homo sapiens CXCL10 | 5 |
| Amino acid sequence of Homo sapiens CXCL10 | 6 |
| mRNA sequence of Homo sapiens CXCL11 | 7 |
| DNA sequence of Homo sapiens CXCL11 | 8 |
| Amino acid sequence of Homo sapiens CXCL11 | 9 |
| mRNA sequence of Homo sapiens prochemerin including a 20 | 10 |
| amino acid hydrophobic signal peptide | |
| DNA sequence of Homo sapiens prochemerin including a 20 | 11 |
| amino acid hydrophobic signal peptide | |
| Amino acid sequence of Homo sapiens prochemerin including | 12 |
| a 20-amino acid hydrophobic signal peptide | |
| mRNA sequence of Homo sapiens prochemerin where the 20 | 13 |
| amino acid hydrophobic signal peptide has been removed | |
| DNA sequence of Homo sapiens prochemerin where the 20 | 14 |
| amino acid hydrophobic signal peptide has been removed | |
| Amino acid sequence of Homo sapiens prochemerin where the | 15 |
| 20 amino acid hydrophobic signal peptide has been removed | |
| mRNA sequence of Homo sapiens mature chemerin with the | 16 |
| signal peptide removed | |
| DNA sequence of Homo sapiens mature chemerin with the | 17 |
| signal peptide removed | |
| Amino acid sequence of Homo sapiens mature chemerin with | 18 |
| the signal peptide removed | |
| mRNA sequence encoding Homo sapiens chemerin that | 19 |
| includes the removal of six amino acids of the chemerin | |
| polypeptide (“chemerin-157”) | |
| DNA sequence encoding Homo sapiens chemerin that includes | 20 |
| the removal of six amino acids of the chemerin polypeptide | |
| (“chemerin-157”) | |
| Amino acid sequence encoding Homo sapiens chemerin that | 21 |
| includes the removal of six amino acids of the chemerin | |
| polypeptide (“chemerin-157”) | |
| mRNA sequence of Mus musculus mature prochemerin | 22 |
| DNA sequence of Mus musculus mature prochemerin | 23 |
| Amino acid sequence of Mus musculus prochemerin | 24 |
| mRNA sequence of Mus musculus mature chemerin | 25 |
| DNA sequence of Mus musculus mature chemerin | 26 |
| Amino acid sequence of Mus musculus chemerin | 27 |
| mRNA sequence of mouse prochemerin sequence comprising | 28 |
| a 20-amino acid hydrophobic signal peptide | |
| DNA sequence of mouse prochemerin sequence comprising a | 29 |
| 20-amino acid hydrophobic signal peptide | |
| Amino acid sequence of mouse prochemerin sequence | 30 |
| comprising a 20-amino acid hydrophobic signal peptide | |
| Amino acid sequence of Homo sapiens chemerin in reverse | 31 |
| orientation (“chemerin-157”). | |
| Nucleotide sequence of alphavirus native subgenomic promoter | 32 |
| Nucleotide sequence of extended subgenomic promoter (v2) | 33 |
| Nucleotide sequence of extended subgenomic promoter (v3) | 34 |
| Nucleotide sequence of wild-type EMCV IRES | 35 |
| Nucleotide sequence of extended subgenomic promoter (v4) | 36 |
| Intentionally skipped | 37 |
| Intentionally skipped | 38 |
| Nucleotide sequence of an interrupting linker | 39 |
| Nucleotide sequence of a GC-rich element | 40 |
| Nucleotide sequence of a GC-rich element | 41 |
| Intentionally skipped | 42 |
| Nucleotide sequence of a histone stem loop | 43 |
| Nucleotide sequence of 5′UTR of VEEV | 44 |
| Nucleotide sequence of 3′UTR of SINV | 45 |
| Nucleotide sequence of extended subgenomic promoter | 46 |
| Poly-A sequence | 47 |
| DNA sequence of self-replicating VEEV mRNA-H5 | 48 |
| RNA sequence of self-replicating VEEV mRNA-H5 | 49 |
| Plasmid sequence of self-replicating VEEV mRNA-H5 | 50 |
| Nucleotide sequence of extended subgenomic promoter | 51 |
| Nucleotide sequence of extended subgenomic promoter | 52 |
| Nucleotide sequence of a GC-rich element | 53 |

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source. Similarly, the term “based on” shall be taken to indicate that a specified integer may be developed or used from a particular source albeit not necessarily directly from that source.

### Selected Definitions

As used herein, the term “self-replicating RNA” refers to a construct based on an RNA virus that has been engineered to allow expression of heterologous RNA and proteins. Self-replicating RNA (e.g., in the form of naked RNA) can amplify in host cells leading to expression of the desired gene product in the host cell.

As used herein, the term “conventional mRNA” or “cRNA” or “non-amplifying RNA” refers to a construct that allows expression of heterologous RNA and proteins but the RNA that cannot amplify in host cells.

As used herein, the term “monocistronic” in reference to the self-replicating RNA, refers to a RNA that encodes one polypeptide.

As used herein, the term “multicistronic” (also known as “polycistronic”) in reference to the polynucleotide, RNA, cRNA and/or self-replicating RNA, refers to a RNA that encodes two or more polypeptides. The term encompasses “bicistronic” (or “dicistronic”; i.e., encoding two polypeptides), “tricistronic” (i.e., encoding three polypeptides) molecules as well as RNA that is capable of encoding more than three polypeptides. By “bicistronic” is meant a single nucleic acid that is capable of encoding two distinct polypeptides from different regions of the nucleic acid.

The term “naked” as used herein refers to nucleic acids that are substantially free of other macromolecules, such as lipids, polymers and proteins. A “naked” nucleic acid, such as a self-replicating RNA, is not formulated with other macromolecules to improve cellular uptake. Accordingly, a naked nucleic acid is not encapsulated in, absorbed on, or bound to a LNP, a liposome, a polymeric microparticle or an oil-in-water emulsion.

As used herein, the term “nucleotide sequence” or “nucleic acid sequence” will be understood to mean a series of contiguous nucleotides (or bases) covalently linked to a phosphodiester backbone. By convention, sequences are presented from the 5′ end to the 3′ end, unless otherwise specified. To facilitate a clear description of the nucleic acids, particular sequence components are referred to as e.g., a “first nucleotide sequence” and a “second nucleotide sequence”. It is to be understood that the first and second sequences can appear in any desired order or orientation, unless otherwise specified, and that no particular order or orientation is intended by the words “first”, “second” etc.

As used herein, the term “chemoattractant” refers to a molecule or structure that attract immune cells, by binding to receptors on leukocytes causing their stimulation, polarization, and locomotion.

As used herein, the term “antigen” refers to a molecule or structure containing one or more epitopes that induce, elicit, augment or boost a cellular and/or humoral immune response. Antigens can include, for example, proteins and peptides from a pathogen such as a virus, bacteria, fungus, protozoan, plant or from a tumour.

The term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

As used herein, the term “operably linked to” means positioning a subgenomic promoter or regulatory element (e.g., an IRES) relative to a nucleic acid such that expression of the nucleic acid is controlled or regulated by the element. For example, a subgenomic promoter can be operably linked to numerous nucleic acids, e.g., through another regulatory element, such as an internal ribosome entry site (IRES).

As used herein, the term “subgenomic promoter” (also known as ‘junction region’ promoter) refers to a promoter that directs the expression of a heterologous nucleotide sequence, regulating protein expression.

As used herein, the term “internal ribosome entry site” or “IRES” refers to a sequence of nucleotides within a mRNA to which a ribosome or a component thereof, e.g., a 40S subunit of a ribosome, is capable of binding. An IRES need not necessarily comprise nucleic acid that induces translation of a mRNA (e.g., a start codon; AUG).

The term “recombinant” shall be understood to mean the product of artificial genetic recombination.

As used herein, the terms “disease”, “disorder” or “condition” refers to a disruption of or interference with normal function, and is not to be limited to any specific condition, and will include diseases or disorders.

As used herein, a subject “at risk” of developing a disease or condition may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the terms “treating”, “treat” or “treatment” include administering a RNA or composition described herein to thereby reduce or eliminate at least one symptom of a specified disease or condition.

As used herein, the term “preventing”, “prevent” or “prevention” includes providing prophylaxis with respect to occurrence or recurrence of a specified disease or condition in an individual. An individual may be predisposed to or at risk of developing the disease but has not yet been diagnosed with the disease.

As used herein, the phrase “delaying progression of” includes reducing or slowing down the progression of the disease or condition in an individual and/or at least one symptom of a disease or condition.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of RNA. The effective amount can be administered in a single dose or in a dose repeated once or several times over a treatment period.

A “therapeutically effective amount” is at least the minimum concentration required to effect a measurable improvement of a particular disease or condition.

A therapeutically effective amount herein may vary according to factors such as the disease state, age, sex, and weight of the patient, and the ability of the RNA of the present disclosure to elicit a desired response in the individual. A therapeutically effective amount is also one in which any toxic or detrimental effects of the RNA are outweighed by the therapeutically beneficial effects.

As used herein, the term “prophylactically effective amount” shall be taken to mean a sufficient quantity of the RNA of the disclosure to prevent or inhibit or delay the onset of one or more detectable symptoms of a disease or disorder as described herein.

As used herein, the term “subject” shall be taken to mean any animal including humans, for example a mammal. Exemplary subjects include but are not limited to humans and non-human primates. For example, the subject is a human.

### Chemoattractants

Chemoattractants are molecules that attract immune cells, by binding to receptors on leukocytes causing their stimulation, polarization, and locomotion. Leukocyte locomotion toward higher concentrations of a chemoattractant is referred to as chemotaxis. Thus, the chemoattractant advantageously attracts immune cells to the site at which an antigen is expressed from the polynucleotide or RNA of the disclosure, thereby enhancing the immunogenicity of the polynucleotide or RNA.

Chemoattractants that can be encoded by a RNA according to the present disclosure will be apparent to the skilled person and include, for example, chemerin, CXCR3 ligands such as CXCL9 (aka MIG), CXCL10 (aka IP-10), CXCL11 (aka I-TAC or IP-9), or other chemokines known to possess chemoattractant properties and that are capable of acting as immunostimulating agents or adjuvants.

In some examples, the chemoattractant is chemerin. Chemerin (also known as retinoic acid receptor responder protein 2 (RARRES2), tazarotene-induced gene 2 protein (TIG2), or RAR-responsive protein TIG2) is a protein that in humans is encoded by the RARRES2 gene. Chemerin is a 14 kDa protein that acts as a ligand for the G protein-coupled receptor CMKLR1 (also known as ChemR23). Chemerin is secreted in an inactive form as prochemerin and is activated through cleavage of the C-terminus by inflammatory and coagulation serine proteases.

The term chemerin includes the active or inactive version of chemerin. The inactive version of chemerin, or pro-form may also be termed “prochemerin.” Prochemerin is activated through cleavage of the C-terminus by inflammatory and coagulation serine proteases. The term chemerin also includes variants of chemerin such a truncated or mutated forms of chemerin, provided the truncated or mutated form of chemerin functions as the active version of chemerin (i.e. chemoattractant). The sequence information for the full length human chemerin amino acid sequence can be found using, for example, the GenBank accession number CAG46789.1 or NP 002880.1. The sequence information for the full length human chemerin gene sequence can be found using, for example, the GenBank Gene ID number 5919 or the full length human chemerin mRNA sequence can be found using, for example, the GenBank accession number CR542026.1 or CR541992.1 or NM_002889.4.

A skilled person will appreciate that chemerin may be found in a variety of species. Non-limiting examples include mouse (NP 018128.1), cattle (NP_001039485.1), boar (NP_001116658.1), goat (NP_001299622.1), rat (NP_001013445.1), chicken (NP_001264405.1), hamster (NP 001231216.1), orangutan (NP 001127631.1), and frog (NP 001135487.1) and the corresponding DNA and mRNA encoding these amino acid sequences.

The present disclosure is also directed to the use of CXCL9 (aka MIG), CXCL10 (aka IP-10), CXCL11 (aka I-TAC or IP-9). These chemokines belong to the CXC chemokine family which play roles in inducing chemotaxis, promoting differentiation and multiplication of leukocytes, and causing tissue extravasation. Each of CXCL9, CXCL10 and CXCL11 act through the chemokine receptor CXCR3 receptor to regulate immune cell migration, differentiation, and activation. Immune reactivity occurs through recruitment of immune cells, such as cytotoxic lymphocytes (CTLs), natural killer (NK) cells, NKT cells, and macrophages. Th1 polarization also activates the immune cells in response to IFN-γ. In the context of the present disclosure, it is envisaged that the use of any of chemerin, CXCL9, CXCL10 or CXCL11 will provide for an adjuvant effect which may include one or more of:
increasing the magnitude or function of the antibody response;
increasing cell mediated immunity;
inducing mucosal immunity; and/or
reducing antigen dose.

The present disclosure provides a nucleic acid encoding a chemerin, CXCL9, CXCL10, CXCL11 for use in vaccine compositions.

In an example, the mRNA sequence encoding a chemoattractant comprises a polynucleotide sequence having at least about 70%, at least about 75%, at least about 80%, at least about 85%, at least about 90% at least about 95%, at least about 96%, at least about 97%, at least about 98%, at least about 99% to a polynucleotide sequence set forth in any one of SEQ ID NOs: 1, 4, 7, 10, 13, 16, 19, 22, 25 or 28.

In an example, the DNA sequence encoding a chemoattractant comprises a polynucleotide sequence having at least about 70%, at least about 75%, at least about 80%, at least about 85%, at least about 90% at least about 95%, at least about 96%, at least about 97%, at least about 98%, at least about 99% to a polynucleotide sequence set forth in any one of SEQ ID NOs: 2, 5, 8, 11, 14, 17, 20, 23, 26 or 29.

In one example, the mRNA sequence encoding a CXCL9 comprises the polynucleotide sequence:

(SEQ ID NO: 1)
AUGAAGAAGAGCGGCGUGCUGUUCCUGCUGGGCAUCAUCCUGCUGG
UGCUGAUCGGCGUGCAGGGCACCCCCGUGGUGAGGAAGGGCAGGU
GCAGCUGCAUCAGCACCAACCAGGGCACCAUCCACCUGCAGAGCC
UGAAGGACCUGAAGCAGUUCGCCCCCAGCCCCAGCUGCGAGAAGA
UCGAGAUCAUCGCCACCCUGAAGAACGGCGUGCAGACCUGCCUGA
ACCCCGACAGCGCCGACGUGAAGGAGCUGAUCAAGAAGUGGGAGA
AGCAGGUGAGCCAGAAGAAGAAGCAGAAGAACGGCAAGAAGCACC
AGAAGAAGAAGGUGCUGAAGGUGAGGAAGAGCCAGAGGAGCAGGC
AGAAGAAGACCACC.

In one example, the DNA sequence encoding a CXCL9 comprises the polynucleotide sequence:

(SEQ ID NO: 2)
ATGAAGAAGAGCGGCGTGCTGTTCCTGCTGGGCATCATCCTGCTG
GTGCTGATCGGCGTGCAGGGCACCCCCGTGGTGAGGAAGGGCAGG
TGCAGCTGCATCAGCACCAACCAGGGCACCATCCACCTGCAGAGC
CTGAAGGACCTGAAGCAGTTCGCCCCCAGCCCCAGCTGCGAGAAG
ATCGAGATCATCGCCACCCTGAAGAACGGCGTGCAGACCTGCCTG
AACCCCGACAGCGCCGACGTGAAGGAGCTGATCAAGAAGTGGGAG
AAGCAGGTGAGCCAGAAGAAGAAGCAGAAGAACGGCAAGAAGCAC
CAGAAGAAGAAGGTGCTGAAGGTGAGGAAGAGCCAGAGGAGCAGG
CAGAAGAAGACCACC.

In one example, the mRNA sequence encoding a CXCL10 comprises the polynucleotide sequence:

(SEQ ID NO: 4)
AUGAACCAGACCGCCAUCCUGAUCUGCUGCCUGAUCUUCCUGACC
CUGAGCGGCAUCCAGGGCGUGCCCCUGAGCAGGACCGUGAGGUGC
ACCUGCAUCAGCAUCAGCAACCAGCCCGUGAACCCCAGGAGCCUG
GAGAAGCUGGAGAUCAUCCCCGCCAGCCAGUUCUGCCCCAGGGUG
GAGAUCAUCGCCACCAUGAAGAAGAAGGGCGAGAAGAGGUGCCUG
AACCCCGAGAGCAAGGCCAUCAAGAACCUGCUGAAGGCCGUGAGC
AAGGAGAGGAGCAAGAGGAGCCCC.

In one example, the DNA sequence encoding a CXCL10 comprises the polynucleotide sequence:

(SEQ ID NO: 5)
ATGAACCAGACCGCCATCCTGATCTGCTGCCTGATCTTCCTGACC
CTGAGCGGCATCCAGGGCGTGCCCCTGAGCAGGACCGTGAGGTGC
ACCTGCATCAGCATCAGCAACCAGCCCGTGAACCCCAGGAGCCTG
GAGAAGCTGGAGATCATCCCCGCCAGCCAGTTCTGCCCCAGGGTG
GAGATCATCGCCACCATGAAGAAGAAGGGCGAGAAGAGGTGCCTG
AACCCCGAGAGCAAGGCCATCAAGAACCTGCTGAAGGCCGTGAGC
AAGGAGAGGAGCAAGAGGAGCCCC.

In one example, the mRNA sequence encoding a CXCL11 comprises the polynucleotide sequence:

(SEQ ID NO: 7)
AUGAGCGUGAAGGGCAUGGCCAUCGCCCUGGCCGUGAUCCUGUGC
GCCACCGUGGUGCAGGGCUUCCCCAUGUUCAAGAGGGGCAGGUGC
CUGUGCAUCGGCCCCGGCGUGAAGGCCGUGAAGGUGGCCGACAUC
GAGAAGGCCAGCAUCAUGUACCCCAGCAACAACUGCGACAAGAUC
GAGGUGAUCAUCACCCUGAAGGAGAACAAGGGCCAGAGGUGCCUG
AACCCCAAGAGCAAGCAGGCCAGGCUGAUCAUCAAGAAGGUGGAG
AGGAAGAACUUC.

In one example, the DNA sequence encoding a CXCL11 comprises the polynucleotide sequence:

(SEQ ID NO: 8)
ATGAGCGTGAAGGGCATGGCCATCGCCCTGGCCGTGATCCTGTGC
GCCACCGTGGTGCAGGGCTTCCCCATGTTCAAGAGGGGCAGGTGC
CTGTGCATCGGCCCCGGCGTGAAGGCCGTGAAGGTGGCCGACATC
GAGAAGGCCAGCATCATGTACCCCAGCAACAACTGCGACAAGATC
GAGGTGATCATCACCCTGAAGGAGAACAAGGGCCAGAGGTGCCTG
AACCCCAAGAGCAAGCAGGCCAGGCTGATCATCAAGAAGGTGGAG
AGGAAGAACTTC.

In one example, the mRNA sequence encoding a chemerin comprises the polynucleotide sequence:

(SEQ ID NO: 10)
AUGAGGAGGCUGCUGAUCCCCCUGGCCCUGUGGCUGGGCGCCGUG
GGCGUGGGCGUGGCCGAGCUGACCGAGGCCCAGAGGAGGGGCCUG
CAGGUGGCCCUGGAGGAGUUCCACAAGCACCCCCCCGUGCAGUGG
GCCUUCCAGGAGACCAGCGUGGAGAGCGCCGUGGACACCCCCUUC
CCCGCCGGCAUCUUCGUGAGGCUGGAGUUCAAGCUGCAGCAGACC
AGCUGCAGGAAGAGGGACUGGAAGAAGCCCGAGUGCAAGGUGAGG
CCCAACGGCAGGAAGAGGAAGUGCCUGGCCUGCAUCAAGCUGGGC
AGCGAGGACAAGGUGCUGGGCAGGCUGGUGCACUGCCCCAUCGAG
ACCCAGGUGCUGAGGGAGGCCGAGGAGCACCAGGAGACCCAGUGC
CUGAGGGUGCAGAGGGCCGGCGAGGACCCCCACAGCUUCUACUUC
CCCGGCCAGUUCGCCUUCAGCAAGGCCCUGCCCAGGAGC.

In one example, the DNA sequence encoding a chemerin comprises the polynucleotide sequence:

(SEQ ID NO: 11)
ATGAGGAGGCTGCTGATCCCCCTGGCCCTGTGGCTGGGCGCCGTG
GGCGTGGGCGTGGCCGAGCTGACCGAGGCCCAGAGGAGGGGCCTG
CAGGTGGCCCTGGAGGAGTTCCACAAGCACCCCCCCGTGCAGTGG
GCCTTCCAGGAGACCAGCGTGGAGAGCGCCGTGGACACCCCCTTC
CCCGCCGGCATCTTCGTGAGGCTGGAGTTCAAGCTGCAGCAGACC
AGCTGCAGGAAGAGGGACTGGAAGAAGCCCGAGTGCAAGGTGAGG
CCCAACGGCAGGAAGAGGAAGTGCCTGGCCTGCATCAAGCTGGGC
AGCGAGGACAAGGTGCTGGGCAGGCTGGTGCACTGCCCCATCGAG
ACCCAGGTGCTGAGGGAGGCCGAGGAGCACCAGGAGACCCAGTGC
CTGAGGGTGCAGAGGGCCGGCGAGGACCCCCACAGCTTCTACTTC
CCCGGCCAGTTCGCCTTCAGCAAGGCCCTGCCCAGGAGC.

SEQ ID NO: 10 and 11 encode human prochemerin comprising a 20-amino acid hydrophobic signal peptide.

In one example, the mRNA sequence encoding a chemerin comprises the polynucleotide sequence:

(SEQ ID NO: 13)
GAGCUGACCGAGGCCCAGAGGAGGGGCCUGCAGGUGGCCCUGGAG
GAGUUCCACAAGCACCCCCCCGUGCAGUGGGCCUUCCAGGAGACC
AGCGUGGAGAGCGCCGUGGACACCCCCUUCCCCGCCGGCAUCUUC
GUGAGGCUGGAGUUCAAGCUGCAGCAGACCAGCUGCAGGAAGAGG
GACUGGAAGAAGCCCGAGUGCAAGGUGAGGCCCAACGGCAGGAAG
AGGAAGUGCCUGGCCUGCAUCAAGCUGGGCAGCGAGGACAAGGUG
CUGGGCAGGCUGGUGCACUGCCCCAUCGAGACCCAGGUGCUGAGG
GAGGCCGAGGAGCACCAGGAGACCCAGUGCCUGAGGGUGCAGAGG
GCCGGCGAGGACCCCCACAGCUUCUACUUCCCCGGCCAGUUCGCC
UUCAGCAAGGCCCUGCCCAGGAGC.

In one example, the DNA sequence encoding a chemerin comprises the polynucleotide sequence:

(SEQ ID NO: 14)
GAGCTGACCGAGGCCCAGAGGAGGGGCCTGCAGGTGGCCCTGGAG
GAGTTCCACAAGCACCCCCCCGTGCAGTGGGCCTTCCAGGAGACC
AGCGTGGAGAGCGCCGTGGACACCCCCTTCCCCGCCGGCATCTTC
GTGAGGCTGGAGTTCAAGCTGCAGCAGACCAGCTGCAGGAAGAGG
GACTGGAAGAAGCCCGAGTGCAAGGTGAGGCCCAACGGCAGGAAG
AGGAAGTGCCTGGCCTGCATCAAGCTGGGCAGCGAGGACAAGGTG
CTGGGCAGGCTGGTGCACTGCCCCATCGAGACCCAGGTGCTGAGG
GAGGCCGAGGAGCACCAGGAGACCCAGTGCCTGAGGGTGCAGAGG
GCCGGCGAGGACCCCCACAGCTTCTACTTCCCCGGCCAGTTCGCC
TTCAGCAAGGCCCTGCCCAGGAGC.

SEQ ID NO: 13 and 14 encode mature human prochemerin with the signal peptide removed.

In one example, the mRNA sequence encoding a chemerin comprises the polynucleotide sequence:

(SEQ ID NO: 16)
CUGACCGAGGCCCAGAGGAGGGGCCUGCAGGUGGCCCUGGAGGAG
UUCCACAAGCACCCCCCCGUGCAGUGGGCCUUCCAGGAGACCAGC
GUGGAGAGCGCCGUGGACACCCCCUUCCCCGCCGGCAUCUUCGUG
AGGCUGGAGUUCAAGCUGCAGCAGACCAGCUGCAGGAAGAGGGAC
UGGAAGAAGCCCGAGUGCAAGGUGAGGCCCAACGGCAGGAAGAGG
AAGUGCCUGGCCUGCAUCAAGCUGGGCAGCGAGGACAAGGUGCUG
GGCAGGCUGGUGCACUGCCCCAUCGAGACCCAGGUGCUGAGGGAG
GCCGAGGAGCACCAGGAGACCCAGUGCCUGAGGGUGCAGAGGGCC
GGCGAGGACCCCCACAGCUUCUACUUCCCCGGCCAGUUCGCCUUC
AGC.

In one example, the DNA sequence encoding a chemerin comprises the polynucleotide sequence:

(SEQ ID NO: 17)
GAGCTGACCGAGGCCCAGAGGAGGGGCCTGCAGGTGGCCCTGGAG
GAGTTCCACAAGCACCCCCCCGTGCAGTGGGCCTTCCAGGAGACC
AGCGTGGAGAGCGCCGTGGACACCCCCTTCCCCGCCGGCATCTTC
GTGAGGCTGGAGTTCAAGCTGCAGCAGACCAGCTGCAGGAAGAGG
GACTGGAAGAAGCCCGAGTGCAAGGTGAGGCCCAACGGCAGGAAG
AGGAAGTGCCTGGCCTGCATCAAGCTGGGCAGCGAGGACAAGGTG
CTGGGCAGGCTGGTGCACTGCCCCATCGAGACCCAGGTGCTGAGG
GAGGCCGAGGAGCACCAGGAGACCCAGTGCCTGAGGGTGCAGAGG
GCCGGCGAGGACCCCCACAGCTTCTACTTCCCCGGCCAGTTCGCC
TTCAGCAAGGCCCTGCCCAGGAGC.

SEQ ID NO: 16 and 17 encode mature human chemerin with the signal peptide removed.

In one example, the mRNA sequence encoding a chemerin comprises the polynucleotide sequence:

(SEQ ID NO: 19)
AUGCGCCGCCUGCUGAUUCCGCUGGCGCUGUGGCUGGGCGCGGUG
GGCGUGGGCGUGGCGGAACUGACCGAAGCGCAGCGCCGCGGCCUG
CAGGUGGCGCUGGAAGAAUUUCAUAAACAUCCGCCGGUGCAGUGG
GCGUUUCAGGAAACCAGCGUGGAAAGCGCGGUGGAUACCCCGUUU
CCGGCGGGCAUUUUUGUGCGCCUGGAAUUUAAACUGCAGCAGACC
AGCUGCCGCAAACGCGAUUGGAAAAAACCGGAAUGCAAAGUGCGC
CCGAACGGCCGCAAACGCAAAUGCCUGGCGUGCAUUAAACUGGGC
AGCGAAGAUAAAGUGCUGGGCCGCCUGGUGCAUUGCCCGAUUGAA
ACCCAGGUGCUGCGCGAAGCGGAAGAACAUCAGGAAACCCAGUGC
CUGCGCGUGCAGCGCGCGGGCGAAGAUCCGCAUAGCUUUUAUUUU
CCGGGCCAGUUUGCGUUUAGC.

In one example, the DNA sequence encoding a chemerin comprises the polynucleotide sequence:

(SEQ ID NO: 20)
ATGCGCCGCCTGCTGATTCCGCTGGCGCTGTGGCTGGGCGCGGTG
GGCGTGGGCGTGGCGGAACTGACCGAAGCGCAGCGCCGCGGCCTG
CAGGTGGCGCTGGAAGAATTTCATAAACATCCGCCGGTGCAGTGG
GCGTTTCAGGAAACCAGCGTGGAAAGCGCGGTGGATACCCCGTTT
CCGGCGGGCATTTTTGTGCGCCTGGAATTTAAACTGCAGCAGACC
AGCTGCCGCAAACGCGATTGGAAAAAACCGGAATGCAAAGTGCGC
CCGAACGGCCGCAAACGCAAATGCCTGGCGTGCATTAAACTGGGC
AGCGAAGATAAAGTGCTGGGCCGCCTGGTGCATTGCCCGATTGAA
ACCCAGGTGCTGCGCGAAGCGGAAGAACATCAGGAAACCCAGTGC
CTGCGCGTGCAGCGCGCGGGCGAAGATCCGCATAGCTTTTATTTT
CCGGGCCAGTTTGCGTTTAGC.

SEQ ID NO: 19 and 20 encode human chemerin sequence includes the removal of six amino acids of the chemerin polypeptide (“chemerin-157”), which in one embodiment, exhibits the highest activity.

In one example, the mRNA sequence encoding a chemerin comprises the polynucleotide sequence:

(SEQ ID NO: 22)
GAGCUGAGCGAGACCCAGAGGAGGAGCCUGCAGGUGGCCCUGGAG
GAGUUCCACAAGCACCCCCCCGUGCAGCUGGCCUUCCAGGAGAUC
GGCGUGGACAGGGCCGAGGAGGUGCUGUUCAGCGCCGGCACCUUC
GUGAGGCUGGAGUUCAAGCUGCAGCAGACCAACUGCCCCAAGAAG
GACUGGAAGAAGCCCGAGUGCACCAUCAAGCCCAACGGCAGGAGG
AGGAAGUGCCUGGCCUGCCUGAAGAUGGACCCCAAGGGCAAGAUC
CUGGGCAGGAUCGUGCACUGCCCCAUCCUGAAGCAGGGCCCCCAG
GACCCCCAGGAGCUGCAGUGCAUCAAGAUCGCCCAGGCCGGCGAG
GACCCCCACGGCUACUUCCUGCCCGGCCAGUUCGCCUUCAGCAGG
GCCCUGAGGACCAAG.

In one example, the DNA sequence encoding a chemerin comprises the polynucleotide sequence:

(SEQ ID NO: 23)
GAGCTGAGCGAGACCCAGAGGAGGAGCCTGCAGGTGGCCCTGGAG
GAGTTCCACAAGCACCCCCCCGTGCAGCTGGCCTTCCAGGAGATC
GGCGTGGACAGGGCCGAGGAGGTGCTGTTCAGCGCCGGCACCTTC
GTGAGGCTGGAGTTCAAGCTGCAGCAGACCAACTGCCCCAAGAAG
GACTGGAAGAAGCCCGAGTGCACCATCAAGCCCAACGGCAGGAGG
AGGAAGTGCCTGGCCTGCCTGAAGATGGACCCCAAGGGCAAGATC
CTGGGCAGGATCGTGCACTGCCCCATCCTGAAGCAGGGCCCCCAG
GACCCCCAGGAGCTGCAGTGCATCAAGATCGCCCAGGCCGGCGAG
GACCCCCACGGCTACTTCCTGCCCGGCCAGTTCGCCTTCAGCAGG
GCCCTGAGGACCAAG.

SEQ ID NO: 22 and 23 encode mature mouse prochemerin.

In one example, the mRNA sequence encoding a chemerin comprises the polynucleotide sequence:

(SEQ ID NO: 25)
GAGCUGAGCGAGACCCAGAGGAGGAGCCUGCAGGUGGCCCUGGAG
GAGUUCCACAAGCACCCCCCCGUGCAGCUGGCCUUCCAGGAGAUC
GGCGUGGACAGGGCCGAGGAGGUGCUGUUCAGCGCCGGCACCUUC
GUGAGGCUGGAGUUCAAGCUGCAGCAGACCAACUGCCCCAAGAAG
GACUGGAAGAAGCCCGAGUGCACCAUCAAGCCCAACGGCAGGAGG
AGGAAGUGCCUGGCCUGCAUCAAGAUGGACCCCAAGGGCAAGAUC
CUGGGCAGGAUCGUGCACUGCCCCAUCCUGAAGCAGGGCCCCCAG
GACCCCCAGGAGCUGCAGUGCAUCAAGAUCGCCCAGGCCGGCGAG
GACCCCCACGGCUACUUCCUGCCCGGCCAGUUCGCCUUCAGC.

In one example, the DNA sequence encoding a chemerin comprises the polynucleotide sequence:

(SEQ ID NO: 26)
GAGCTGAGCGAGACCCAGAGGAGGAGCCTGCAGGTGGCCCTGGAG
GAGTTCCACAAGCACCCCCCCGTGCAGCTGGCCTTCCAGGAGATC
GGCGTGGACAGGGCCGAGGAGGTGCTGTTCAGCGCCGGCACCTTC
GTGAGGCTGGAGTTCAAGCTGCAGCAGACCAACTGCCCCAAGAAG
GACTGGAAGAAGCCCGAGTGCACCATCAAGCCCAACGGCAGGAGG
AGGAAGTGCCTGGCCTGCATCAAGATGGACCCCAAGGGCAAGATC
CTGGGCAGGATCGTGCACTGCCCCATCCTGAAGCAGGGCCCCCAG
GACCCCCAGGAGCTGCAGTGCATCAAGATCGCCCAGGCCGGCGAG
GACCCCCACGGCTACTTCCTGCCCGGCCAGTTCGCCTTCAGC.

SEQ ID NO: 25 and 26 encode mature mouse chemerin.

In one example, the mRNA sequence encoding a chemerin comprises the polynucleotide sequence:

(SEQ ID NO: 28)
AUGAAGUGCCUGCUGAUCAGCCUGGCCCUGUGGCUGGGCACCGUG
GGCACCAGGGGCACCGAGCCCGAGCUGAGCGAGACCCAGAGGAGG
AGCCUGCAGGUGGCCCUGGAGGAGUUCCACAAGCACCCCCCCGUG
CAGCUGGCCUUCCAGGAGAUCGGCGUGGACAGGGCCGAGGAGGUG
CUGUUCAGCGCCGGCACCUUCGUGAGGCUGGAGUUCAAGCUGCAG
CAGACCAACUGCCCCAAGAAGGACUGGAAGAAGCCCGAGUGCACC
AUCAAGCCCAACGGCAGGAGGAGGAAGUGCCUGGCCUGCAUCAAG
AUGGACCCCAAGGGCAAGAUCCUGGGCAGGAUCGUGCACUGCCCC
AUCCUGAAGCAGGGCCCCCAGGACCCCCAGGAGCUGCAGUGCAUC
AAGAUCGCCCAGGCCGGCGAGGACCCCCACGGCUACUUCCUGCCC
GGCCAGUUCGCCUUCAGC.

In one example, the DNA sequence encoding a chemerin comprises the polynucleotide sequence:

(SEQ ID NO: 29)
ATGAAGTGCCTGCTGATCAGCCTGGCCCTGTGGCTGGGCACCGTG
GGCACCAGGGGCACCGAGCCCGAGCTGAGCGAGACCCAGAGGAGG
AGCCTGCAGGTGGCCCTGGAGGAGTTCCACAAGCACCCCCCCGTG
CAGCTGGCCTTCCAGGAGATCGGCGTGGACAGGGCCGAGGAGGTG
CTGTTCAGCGCCGGCACCTTCGTGAGGCTGGAGTTCAAGCTGCAG
CAGACCAACTGCCCCAAGAAGGACTGGAAGAAGCCCGAGTGCACC
ATCAAGCCCAACGGCAGGAGGAGGAAGTGCCTGGCCTGCATCAAG
ATGGACCCCAAGGGCAAGATCCTGGGCAGGATCGTGCACTGCCCC
ATCCTGAAGCAGGGCCCCCAGGACCCCCAGGAGCTGCAGTGCATC
AAGATCGCCCAGGCCGGCGAGGACCCCCACGGCTACTTCCTGCCC
GGCCAGTTCGCCTTCAGC.

SEQ ID NO: 28 and 29 encode a mouse prochemerin sequence comprising a 20-amino acid hydrophobic signal peptide.

In an example, chemerin has a sequence comprising at least 70% identity to SEQ ID NO: 10, SEQ ID NO: 13, SEQ ID NO:16, SEQ ID NO:19, SEQ ID NO: 22, SEQ ID NO: 25 or SEQ ID NO:28. For example, the chemerin may have about 70%, about 71%, about 72%, about 73%, about 74%, about 75%, 76%, 77%, 78%, 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to any one of SEQ ID NO: 10, SEQ ID NO: 13, SEQ ID NO:16, SEQ ID NO:19, SEQ ID NO: 22, SEQ ID NO: 25 or SEQ ID NO:28.

The present disclosure also provides immunogenic and pharmaceutical compositions comprising a polynucleotide, RNA, cRNA or self-replicating RNA of the disclosure and a chemoattractant polypeptide, optionally selected from the group consisting of chemerin, C-X-C motif chemokine ligand 9 (CXCL9), C-X-C motif chemokine ligand 10 (CXCL10) and C-X-C motif chemokine ligand 11 (CXCL11).

In an example, the chemoattractant polypeptide comprises an amino acid sequence having at least about 70%, at least about 75%, at least about 80%, at least about 85%, at least about 90% at least about 95%, at least about 96%, at least about 97%, at least about 98%, at least about 99% or about 100% identity to any one of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27 or SEQ ID NO: 30.

In an example, the chemerin, CXCL9, CXCL10 or CXCL11 polypeptides comprise an amino acid sequence at least 70% identity to any one of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27 or SEQ ID NO: 30. For example, the chemerin, CXCL9, CXCL10 or CXCL11 polypeptides may have about 70%, about 71%, about 72%, about 73%, about 74%, about 75%, 76%, 77%, 78%, 79%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27 or SEQ ID NO: 30.

In one example, the CXCL9 polypeptide comprises the amino acid sequence:

(SEQ ID NO: 3)
MKKSGVLFLLGIILLVLIGVQGTPVVRKGRCSCISTNQGTIHLQS
LKDLKQFAPSPSCEKIEIIATLKNGVQTCLNPDSADVKELIKKWE
KQVSQKKKQKNGKKHQKKKVLKVRKSQRSRQKKTT.

In one example, the CXCL10 polypeptide comprises the amino acid sequence:

(SEQ ID NO: 6)
MNQTAILICCLIFLTLSGIQGVPLSRTVRCTCISISNQPVNPRSL
EKLEIIPASQFCPRVEIIATMKKKGEKRCLNPESKAIKNLLKAVS
KERSKRSP.

In one example, the CXCL11 polypeptide comprises the amino acid sequence:

(SEQ ID NO: 9)
MSVKGMAIALAVILCATVVQGFPMFKRGRCLCIGPGVKAVKVADI
EKASIMYPSNNCDKIEVIITLKENKGQRCLNPKSKQARLIIKKVE
RKNF.

An exemplary chemoattractant is chemerin. In some examples, the chemerin comprises an amino acid sequence having at least about 70%, at least about 75%, at least about 80%, at least about 85%, at least about 90% at least about 95%, at least about 96%, at least about 97%, at least about 98%, at least about 99% or about 100% identity to any one of SEQ ID NO: 12, SEQ ID NO: 15, SEQ ID NO: 18, SEQ ID NO: 21, SEQ ID NO: 24, SEQ ID NO: 27, SEQ ID NO: 30, or SEQ ID NO: 31.

In one example, the chemerin polypeptide comprises the amino acid sequence:

(SEQ ID NO: 12)
MRRLLIPLALWLGAVGVGVAELTEAQRRGLQVALEEFHKHPPVQW
AFQETSVESAVDTPFPAGIFVRLEFKLQQTSCRKRDWKKPECKVR
PNGRKRKCLACIKLGSEDKVLGRLVHCPIETQVLREAEEHQETQC
LRVQRAGEDPHSFYFPGQFAFSKALPRS.

This sequence is human prochemerin comprising a 20-amino acid hydrophobic signal peptide.

In another example, the chemerin polypeptide comprises the amino acid sequence:

(SEQ ID NO: 15)
ELTEAQRRGLQVALEEFHKHPPVQWAFQETSVESAVDTPFPAGIF
VRLEFKLQQTSCRKRDWKKPECKVRPNGRKRKCLACIKLGSEDKV
LGRLVHCPIETQVLREAEEHQETQCLRVQRAGEDPHSFYFPGQFA
FSKALPRS.

This sequence is mature human prochemerin with the signal peptide removed.

In another example, the chemerin polypeptide comprises the amino acid sequence:

(SEQ ID NO: 18)
LTEAQRRGLQVALEEFHKHPPVQWAFQETSVESAVDTPFPAGIFV
RLEFKLQQTSCRKRDWKKPECKVRPNGRKRKCLACIKLGSEDKVL
GRLVHCPIETQVLREAEEHQETQCLRVQRAGEDPHSFYFPGQFAF
S.

This sequence is mature human chemerin with the signal peptide removed.

In another example, the chemerin polypeptide comprises the amino acid sequence:

(SEQ ID NO: 21)
MRRLLIPLALWLGAVGVGVAELTEAQRRGLQVALEEFHKHPPVQW
AFQETSVESAVDTPFPAGIFVRLEFKLQQTSCRKRDWKKPECKVR
PNGRKRKCLACIKLGSEDKVLGRLVHCPIETQVLREAEEHQETQC
LRVQRAGEDPHSFYFPGQFAFS.

This human sequence includes the removal of six amino acids of the chemerin polypeptide (“chemerin-157”), which in one embodiment, exhibits the highest activity.

In another example, the chemerin comprises the amino acid sequence:

(SEQ ID NO: 24)
ELSETQRRSLQVALEEFHKHPPVQLAFQEIGVDRAEEVLFSAGTF
VRLEFKLQQTNCPKKDWKKPECTIKPNGRRRKCLACIKMDPKGKI
LGRIVHCPILKQGPQDPQELQCIKIAQAGEDPHGYFLPGQFAFSR
ALRTK.

This sequence is mature mouse prochemerin.

In another example, the chemerin polypeptide comprises the amino acid sequence:

(SEQ ID NO: 27)
ELSETQRRSLQVALEEFHKHPPVQLAFQEIGVDRAEEVLFSAGTF
VRLEFKLQQTNCPKKDWKKPECTIKPNGRRRKCLACIKMDPKGKI
LGRIVHCPILKQGPQDPQELQCIKIAQAGEDPHGYFLPGQFAFS.

This sequence is mature mouse chemerin.

In another example, the chemerin polypeptide comprises the amino acid sequence:

(SEQ ID NO: 30)
MKCLLISLALWLGTVGTRGTEPELSETQRRSLQVALEEFHKHPPV
QLAFQEIGVDRAEEVLFSAGTFVRLEFKLQQTNCPKKDWKKPECT
IKPNGRRRKCLACIKMDPKGKILGRIVHCPILKQGPQDPQELQCI
KIAQAGEDPHGYFLPGQFAFS.

This sequence is a mouse prochemerin sequence comprising a 20-amino acid hydrophobic signal peptide.

In another example, a suitable control chemerin polypeptide comprises the amino acid sequence:

(SEQ ID NO: 31)
MRRLLIPLALWLGAVGVGVAELTEAQRRGLQVALEEFHKHPPVQW
AFQETSVESAVDTPFPAGIFVRLEFKLQQTSCRKRDWKKPECKVR
PNGRKRKCLACIKLGSEDKVLGRLVHCPIETQVLREAEEHQETQC
LRVQRAGEDPHSFSFAFQGPFY*.

This sequence is a reverse orientation of human chemerin (“chemerin-157”).

In certain embodiments, the chemerin, CXCL9, CXCL10 or CXCL11 polypeptides may be PEGylated for improved systemic half-life and reduced dosage frequency. In an embodiment, PEG may be added to the chemerin, CXCL9, CXCL10 or CXCL11 polypeptides. As such, a composition of the disclosure may comprise chemerin, CXCL9, CXCL10 or CXCL11 polypeptides comprising PEG. In an embodiment, PEG may be selected from the group consisting of PEG-10K, PEG-20K and PEG-40K. Methods of conjugating PEG to a protein are standard in the art. For example, see Kolate et al, Journal of Controlled Release 2014; 192(28): 67-81, which is hereby incorporated by reference in its entirety.

It is appreciated that the present disclosure is directed to analogs of chemerin, CXCL9, CXCL10 and CXCL11 in other organisms and is not limited to the human analog. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the disclosure.

Equally, BLAST protein searches may be performed with the BLASTX program to obtain amino acid sequences that are homologous to a polypeptide of the disclosure. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed.

### Antigens

Antigens suitable for use in accordance with the present disclosure will be apparent to the skilled person and, for example, include proteins and peptides derived from any pathogen. For example, the antigen is a virus, bacteria, a fungus or a protozoan.

### Viral Antigens

In one example, the antigen is a viral antigen.

Viral antigens that can be encoded by a RNA according to the present disclosure will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxovirus types 1-4 (PIV), Mumps, Sendai viruses, Simian virus 5)), Bovine parainfluenza virus, Nipahvirus, Henipavirus and Newcastle disease virus), Poxviridae (e.g., Variola vera, including but not limited to, Variola major and Variola minor, Metapneumoviruses, such as human metapneumovirus (hMPV) and avian metapneumoviruses (aMPV)), Morbilliviruses (e.g., Measles), Picornaviruses (e.g., Enteroviruses, Rhinoviruses, Heparnavirus, Parechovirus, Cardioviruses and Aphthoviruses), Enteroviruseses (e.g., Poliovirus types 1, 2 or 3, Coxsackie A virus types 1 to 22 and 24, Coxsackie B virus types 1 to 6, Echovirus (ECHO) virus types 1 to 9, 11 to 27 and 29 to 34 and Enterovirus 68 to 71), Bunyaviruses (e.g., California encephalitis virus), Phlebovirus (e.g., Rift Valley Fever virus), Nairovirus (e.g., Crimean-Congo hemorrhagic fever virus), Heparnaviruses (e.g., Hepatitis A virus (HAV)), Togaviruses (e.g., Rubivirus, an Alphavirus, or an Arterivirus), Flaviviruses (e.g., Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus), Pestiviruses (e.g., Bovine viral diarrhea (BVDV), Classical swine fever (CSFV) or Border disease (BDV)), Hepadnaviruses (e.g., Hepatitis B virus, Hepatitis C virus), Rhabdoviruses (e.g., Lyssavirus (Rabies virus) and Vesiculovirus (VSV)), Caliciviridae (e.g., Norwalk virus, and Norwalk-like Viruses (e.g., Hawaii Virus and Snow Mountain Virus); Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV), and Porcine transmissible gastroenteritis virus (TGEV)), Retroviruses (e.g., Oncovirus, a Lentivirus or a Spumavirus), Reoviruses (e.g., Orthoreo virus, a Rotavirus, an Orbivirus, or a Coltivirus), Parvoviruses (e.g., Parvovirus B 19), Delta hepatitis virus (HDV), Hepatitis E virus (HEV), Human Herpesviruses (e.g., Herpes Simplex Viruses (HSV), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8)), Papovaviruses (e.g., Papillomaviruses and Polyomaviruses), Adenoviruess and Arenaviruses.

In one example, the antigen is a viral antigen from a respiratory virus. Respiratory viral antigens that can be encoded by the self-replicating RNA will be apparent to the skilled person and include, for example, proteins and peptides from a Orthomyxoviruses (e.g., Influenza A, B and C), Paramyxoviridae viruses (Pneumoviruses (e.g., Respiratory syncytial virus (RSV), Bovine respiratory syncytial virus, Pneumonia virus of mice, and Turkey rhinotracheitis virus), Paramyxoviruses (PIV), and Metapneumovirus such as human metapneumovirus (hMPV) and avian metapneumoviruses (aMPV)), Picornaviruses (e.g., Rhinoviruses) and Coronaviruses (e.g., severe acute respiratory syndrome (SARS) coronavirus (SARS-CoV), SARS coronavirus 2 (SARS-CoV-2), Middle East respiratory syndrome (MERS) coronavirus (MERS-CoV), Avian infectious bronchitis (IBV), Mouse hepatitis virus (MHV)).

In one example, the viral antigen is from an influenza virus.

In one example, the viral antigen is from a respiratory syncytial virus. RSV is an enveloped non-segmented negative-strand RNA virus in the family Paramyxoviridae, genus Pneumovirus. To infect a host cell, paramyxoviruses such as RSV, like other enveloped viruses such as influenza virus and HIV, require fusion of the viral membrane with a host cell's membrane. For RSV, the conserved fusion protein (RSV-F glycoprotein) fuses the viral and cellular membranes by coupling irreversible protein refolding with juxtaposition of the membranes. In current models based on paramyxovirus studies, the RSV-F protein initially folds into a metastable pre-fusion conformation. During cell entry, the pre-fusion conformation undergoes refolding and conformational changes to its stable post fusion conformation.

In one example, the antigen may be derived from the RSV surface glycoproteins Fusion (F), Glycoprotein (G), Small Hydrophobic protein (SH), the matrix proteins M and M2, the nucleocapsid proteins N., P and L, and the nonstructural proteins NS1 and NS2.

RSV-F antigens suitable for inclusion in the immunogenic compositions described herein, either in RNA encoded form or as polypeptides, include RSV-F glycoprotein and RSV-F glycoprotein variants. Suitable RSV-F glycoprotein variants include, for example, full length F protein and truncated variants such as soluble ecto-domains, each optionally containing one or more mutations, such as furin cleavage mutations, trypsin-cleavage mutations, fusion peptide mutations (e.g., deletions in whole or in part), mutations that stabilize the HRB trimer, and mutations that destabilize the HRA trimer. Full length and truncated RSV-F glycoprotiens, including those with one or more Such mutations in a variety of combinations are well known in the art and are disclosed for example in WO2011/008974, the disclosure of which is incorporated herein by reference in its entirety.

In one example, the viral antigen is from a parainfluenza virus.

In one example, the viral antigen is from a metapneumovirus.

In one example, the viral antigen is from a rhinovirus.

In one example, the viral antigen is from a coronavirus. In this example, the coronavirus may be SARS-CoV2 and the antigen is a RNA encoding a SARS-CoV2 spike glycoprotein peptide or fragment thereof, a RNA encoding a SARS-CoV2 nucleocapsid phosphoprotein peptide or fragment thereof, a RNA encoding a SARS-CoV2 membrane glycoprotein peptide or fragment thereof, a RNA encoding a SARS-CoV2 orf3a or fragment thereof, a RNA encoding a SARS-CoV2 orflab or fragment thereof or a variant of the above.

In one example, the viral antigen is from an adenovirus.

In one example, the viral antigen is from a bocavirus.

In one example, the antigen is from a single strain of an influenza virus (i.e., monovalent) or from multiple strains (i.e., multivalent).

In one example, the antigen is from an influenza A, B and/or C virus strain.

In one example, the antigen is from an influenza A virus strain. For example, the antigen is an influenza A virus hemagglutinin (HA) protein, a neuraminidase (NA) protein, a matrix (M) protein, a nucleoprotein (NP), a non-structural (NS) protein, or an immunogenic fragment or variant thereof. In one example, the antigen is an influenza A hemagglutinin (HA) subtype H1, H2, H3, H4, H5, H6, H7, H8, H9, H10, H11, H12, H13, H14, H15 or H16 and/or an influenza A neuraminidase (NA) subtype N1, N2, N3, N4, N5, N6, N7, N8 or N9 and/or an influenza A matrix (M) protein subtype M1 or M2 and/or an influenza A non-structural (NS) protein subtype NS1 or NS2.

In one example, the influenza viral antigens are from different subtypes of the influenza virus. For example, different hemagglutinin subtypes and/or different neuraminidase subtypes and/or matrix protein subtypes, and/or nucleoprotein subtypes and/or non-structural protein subtypes.

The skilled person will be aware that pandemic strains of the influenza virus are commonly H1, H2, H3, H5, H6, H7 or H9 subtype influenza A virus strains. For example, H1N1, H2N2, H3N2, H5N1, H5N3, H6N1, H7N2, H7N3, H7N7, H7N9 and H9N2, strains.

In one example, the antigen is from influenza A virus strain having the same hemagglutinin subtypes. In another example, the antigen is from influenza A virus strain having different hemagglutinin subtypes. In one example, the antigen is H1, H2, H3, H5, H6, H7 or H9 subtype influenza A virus strain. For example, the antigen is a H1 hemagglutinin, or a H2 hemagglutinin, or a H3 hemagglutinin, or a H5 hemagglutinin, or a H6 hemagglutinin, or a H7 hemagglutinin or a H9 hemagglutinin. For example, the antigen is a H5 subtype influenza A virus strain (i.e., a H5 hemagglutinin). In one example, the H5 hemagglutinin is an A/turkey/Turkey/1/2005 virus strain. In one example, the H3 hemagglutinin is an A/Delaware/39/2019 virus strain.

In one example, the antigen is from influenza A virus strain having the same neuraminidase subtypes. In another example, the antigen is from influenza A virus strains having different neuraminidase subtypes. In one example, the antigen is N1, N2, N3, N7 or N9 subtype influenza A virus strain. For example, the antigen is a N1 neuraminidase, or a N2 neuraminidase, or a N3 neuraminidase, or a N7 neuraminidase, or a N9 neuraminidase. For example, the antigen is a N1 neuraminidase subtype influenza A virus strain. In one example, the N1 neuraminidase is an A/turkey/Turkey/1/2005 strain. In one example, the N2 neuraminidase is an A/Delaware/39/2019 virus strain.

In one example, compositions of the disclosure comprise one or more antigens. In an example, the one or more antigens are a H5 hemagglutinin protein and/or a N1 neuraminidase protein. For example, the first antigen is a H5 hemagglutinin subtype influenza A virus strain and the second antigen is a N1 neuraminidase subtype influenza A virus strain.

Infections such as influenza and coronavirus infection are leading causes of ARDS. Accordingly, in one example of the present disclosure, the ARDS is associated with an influenza or a coronavirus infection, such as a SARS-CoV infection. In one example, the ARDS is associated with a SARS-CoV-2 infection. Thus, a skilled person will understand that antigens targeting a SARS-CoV-2 infection or influenza, including those listed above, may be antigens suitable for the treatment of ARDS.

### Bacterial Antigens

In one example, the antigen is a bacterial antigen.

Suitable bacterial antigens will be apparent to the skilled person and include, for example, proteins and peptides from a Neisseria meningitides, Streptococcus pneumoniae, Streptococcus pyogenes, Moraxella catarrhalis, Bordetella pertussis, Burkholderia sp. (e.g., Burkholderia mallei, Burkholderia pseudomallei and Burkholderia cepacia), Staphylococcus aureus, Haemophilus influenzae, Clostridium tetani (Tetanus), Clostridium perfringens, Clostridium botulinums, Cornynebacterium diphtheriae (Diphtheria), Pseudomonas aeruginosa, Legionella pneumophila, Coxiella burnetii, Brucella sp. (e.g., B. abortus, B. canis, B. melitensis, B. neotomae, B. ovis, B. suis and B. pinnipediae), Francisella sp. (e.g., F. novicida, F. philomiragia and F. tularensis), Streptococcus agalactiae, Neiserria gonorrhoeae, Chlamydia trachomatis, Treponema pallidum (Syphilis), Haemophilus ducreyi, Enterococcus faecalis, Enterococcus faecium, Helicobacter pylori, Staphylococcus saprophyticus, Yersinia enterocolitica, E. coli, Bacillus anthracis (anthrax), Yersinia pestis (plague), Mycobacterium tuberculosis, Rickettsia, Listeria, Chlamydia pneumoniae, Vibrio cholerae, Salmonella typhi (typhoid fever), Borrelia burgdorfer, Porphyromonas sp, Klebsiella sp.

### Fungal Antigens

In one example, the antigen is a fungal antigen.

Suitable fungal antigens will be apparent to the skilled person and include, for example, proteins and peptides from Dermatophytes (including Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T verrucosum var. album, var. discoides, var. ochraceum, Trichophyton violaceum, and/or Trichophyton faviforme), Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi.

### Protazoan Antigens

In one example, the antigen is a protazoan antigen.

Suitable protazoan antigens will be apparent to the skilled person and include, for example, proteins and peptides from Entamoeba histolytica, Giardia lambli, Cryptosporidium parvum, Cyclospora cayatanensis and Toxoplasma.

### Other Molecules

In one example, compositions according to the disclosure comprise an additional RNA encoding: (i) one or more antigens; (ii) one or more immunopotentiating agents; (iii) one or more chemoattractants; and/or (iv) one or more targeting molecules.

As used herein, “immunopotentiating agent” means any molecule capable of enhancing an immune response in a subject. Examples of immunopotentiating agents include cytokines, chemokines, and immune potentiators. In example, the one or more immunopotentiating agents are selected from the group consisting of interleukin 12, interleukin 7, interleukin 15, and interleukin 21.

As used herein, “targeting molecule” means any molecule capable of targeting an immune cell surface marker, for example, a dendritic cell or an antigen presenting cell. In one example, the targeting molecule targets a dendritic cell surface marker. In an example, the targeting molecule is selected from the group consisting of DEC-205, Clec-9A, DC-SIGN, CD11c, DCIR2, Dectin-1/2, CD80/86, F4/80, CIRE, mannose, and CD36. In an example, the targeting molecule is DEC-205.

In another example, the targeting molecule is an antibody. In one example, the targeting molecule is a monoclonal antibody.

### Polynucleotides

As used herein, the term “polynucleotide” refers a molecular chain of nucleotides chemically bonded by a series of ester linkages between the phosphoryl group of one nucleotide and the hydroxyl group of the sugar in an adjacent nucleotide. In one example, the polynucleotide is a DNA. In one example, the polynucleotide is a RNA, e.g., mRNA. For example, the mRNA is a conventional mRNA (cRNA) or a self-replicating RNA.

As used herein, the term “fragment” refers to a portion of a nucleotide sequence or polypeptide of a reference nucleotide sequence or polypeptide disclosed herein which maintains a defined activity of the full length nucleotide sequence or polypeptide.

As used herein, the term “variant” refers to a nucleotide sequence with one or more substitutions, insertions, deletions and/or other modifications compared to the unmodified sequence. It will be apparent to the skilled person that any variant described herein will have the same or similar expression of the encoded protein. For example, the variant is a functional variant. Exemplary modifications to the nucleotide sequence and/or polypeptide will be apparent to the skilled person and/or described herein.

The polynucleotides described herein may comprise a modification. In one example, a modification is a chemical modification of one or more nucleotide(s) of the nucleotide sequence. For example, at least one naturally occurring nucleotide of the polynucleotide is replaced with a chemically modified nucleotide (e.g. pseudouridine (ψ), and 1-methylpseudouridine (m1ψ)).

In one example, the modification comprises increasing the G/C content of the nucleotide sequence.

In one example, the modification comprises codon optimization of the nucleotide sequence.

As used herein, the term “encode”, “encodes” or “encoding” refers to a region of a polynucleotide capable of undergoing translation into a polypeptide.

The polynucleotide of the present disclosure includes DNA and RNA (e.g. mRNA).

### Deoxyribonucleic Acid (DNA)

In one example, the polynucleotide is a DNA (e.g. DNA vector).

It will be apparent to the skilled person that a DNA of the present disclosure may further comprise an endonuclease restriction site at the 3′ end of the 3′UTR. The skilled person will appreciate that endonuclease restriction site allows for the insertion of one or more nucleotide sequence(s) (e.g. encoding an antigen of interest, a fragment and/or a variant thereof) without disrupting the remainder of the DNA.

As used herein, the term “restriction endonuclease site” refers to a sequence of DNA that binds to a restriction endonuclease. Typically, the restriction endonuclease site is short sequence (e.g. of approximately 4-8 base pairs) recognised and cleaved by the restriction endonuclease.

As used herein, the term “restriction enzymes” or “restriction endonucleases” refers to a class of enzyme that occur naturally in bacteria and in some viruses. Restriction endonuclease bind specifically to and cleave double-stranded DNA at specific sites within or adjacent to a restriction endonuclease site. Exemplary restriction endonuclease include, for example, BciVI (Bful), Bcul (Spel), EcoRI, Aat|I, Agel (BshTI), Apal, BamHI, Bglll, BIpl (Bpu11021), BsrGI (Bsp1407), Clal (Bsu151), EcoRI, EcoRV (Eco321), Eam11041 (Earl), HindlIl, Kpnl, MIul, Ncol, Ndel, Nhel, Notl, Nsil, Mph11031), Pstl, Pvul, Pvull, Sac, Sall, Scal, Spel, Xbal, Xhol SaclI (Cfr421) and Xbal.

In one example, the present disclosure provides a transcribable polynucleotide comprising the first nucleotide sequence encoding a first antigen of interest; and a second nucleotide sequence encoding the chemoattractant, e.g., operably linked to a regulatory element selected from the group consisting of a SG promoter and an IRES. For example, the polynucleotide is the DNA plasmid comprising the first and second nucleotide sequences and optionally one or more nucleotide sequence(s) encoding one or more antigens of interest.

In one example, the DNA comprises a nucleotide sequence comprising a restriction endonuclease site located 3′ of the 3′UTR. The presence of the restriction endonuclease site located 3′ of the 3′UTR allows for production of a linearised DNA. Linearisation of DNA ensures defined termination of in vitro transcribed DNA to produce mRNA.

### RNA

The present disclosure relates to RNA, for example an mRNA, and other polynucleotides comprising a nucleotide sequence encoding at least one chemoattractant and at least one antigen as disclosed herein.

In some examples, the polynucleotide is an RNA. As used herein, the term “RNA (ribonucleic acid)” refers to a single stranded molecular chain of nucleotides chemically bonded by a series of ester linakges between the phosphoryl group of one nucleotide and the hydroxyl group of the sugar in an adjacent nucleotide. Suitable forms ofRNA will be apparent to the skilled person. In one example, the RNA is messenger RNA (mRNA). In one example, the mRNA encoding the chemoattract and/or the antigen is a monocistronic mRNA. For example, the monocistronic mRNA is a conventional mRNA (cRNA) or a self-replicating RNA. In another example, the mRNA encoding the chemoattract and/or the antigen is a multicistronic mRNA. For example, the multicistronic mRNA is a conventional mRNA (cRNA) or a self-replicating RNA.

### Conventional (Non-Replicating) RNA

RNAs of the present disclosure encompass a non-replicating mRNA (also referred to as conventional mRNA (cRNA) or non-amplifying). The skilled person will understand that the cRNA of the present disclosure comprise in order from 5′ to 3′: a 5′cap structure, a 5′-UTR, a fragment and/or a variant thereof, a first nucleotide sequence encoding a first antigen of interest, a second nucleotide sequence encoding a second antigen of interest, a 3′-UTR and a 3′tailing sequence (e.g. a polyadenylation signal or one or more poly-A tails). The cRNA of the present disclosure may further comprise a translation internal ribosome entry site (e.g. Kozak consensus sequence or IRES) operably linked to the chemottractant or antigen of interest. In an example, the present disclosure provides a monocistronic cRNA. In an example, the present disclosure provides a multicistronic cRNA.

### Self-Replicating RNA

RNAs of the present disclosure encompass a self-replicating RNA (also known as self-amplifying RNA or sa-mRNA). In an example, the RNA is a self-replicating RNA (also known as a replicon). In an example, the present disclosure provides a monocistronic self-replicating RNA. In an example, the present disclosure provides a multicistronic self-replicating RNA.

The skilled person will understand that the self-replicating RNA of the present disclosure is based on the genomic RNA of RNA viruses. The RNA should be positive (+)-stranded so that it can be directly translated after delivery to a cell without the need for intervening replication steps (e.g., reverse transcription). Translation of the RNA results in the production of non-structural proteins (NSPs) which combine to form a replicase complex (i.e., an RNA-dependent RNA polymerase). The complex then amplifies the original RNA, producing both antisense and sense transcripts, resulting in production of multiple daughter RNAs which may subsequently be translated and transcribed, enhancing overall protein expression.

In one example, the self-replicating RNA of the present disclosure comprises the non-structural proteins of the RNA virus, the 5′ and 3′ untranslated regions (UTRs) and the native subgenomic promoter.

In one example, the self-replicating RNA comprises one or more non-structural proteins of the RNA virus. For example, the RNA comprises at least one or more genes selected from the group consisting of a viral replicase (or viral polymerase), a viral protease, a viral helicase and other non-structural viral proteins. For example, the self-replicating RNA comprises a viral replicase (or viral polymerase).

It will be apparent to the skilled person that RNA suitable for use in the present disclosure may also include a 5′ untranslated region (5′-UTR), a 3′ untranslated region (3′UTR), and/or a coding or translating sequence. In addition, the RNA may comprise a 5′ cap structure, a chain terminating nucleotide, a stem loop (e.g., a histone stem loop), a 3′ tailing sequence (e.g., a polyadenylation signal or one or more polyA tails. In another example, the self-replicating RNA comprises a 5′- and a 3′-end UTR of the RNA virus. It will be apparent to the skilled person that the terms 5′ and a 3′UTR also encompasses the terms 5′ and 3′ conserved sequence elements (CSE). In one example, the self-replicating RNA comprises a 5′- and a 3′-end CSE.

The self-replicating RNA of the present disclosure cannot induce production of infectious viral particles. For example, the self-replicating RNA of the present disclosure does not comprise viral genes encoding structural proteins necessary for production of viral particles.

In one example, the self-replicating RNA is derived from or based on an alphavirus. Suitable alphaviruses will be apparent to the skilled person and/or described herein.

In another example, the self-replicating RNA is derived from or based on a virus other than an alphavirus, for example, a positive-stranded RNA virus. Suitable positive-stranded RNA viruses suitable for use in the present disclosure will be apparent to the skilled person and include, for example, a picornavirus, a flavivirus, a rubivirus, a pestivirus, a hepacivirus, a calicivirus, or a coronavirus.

### Alphavirus

In one example, the self-replicating RNA of the present disclosure is derived from (or based on) an alphavirus.

Alphaviruses are the sole genus in the Togaviridae family and are an enveloped virus with a positive-sense, single-stranded RNA genome. The skilled person will understand that the alphavirus genome comprises two open reading frames (ORFs), non-structural and structural. The first ORF encodes four non-structural proteins (NSP1, NSP2, NSP3 and NSP4) necessary for transcription and replication of viral RNA. The second encodes three structural proteins: the core nucleocapsid protein C, and the envelope proteins P62 and E1, which associate as a heterodimer. The viral membrane-anchored surface glycoproteins are responsible for receptor recognition and entry into target cells through membrane fusion.

In one example, the self-replicating RNA of the present disclosure comprises a viral replicase (or viral polymerase). For example, the viral replicase is an alphavirus replicase, such as an alphavirus protein NSP4.

In one example, the self-replicating RNA of the present disclosure does not encode one or more alphavirus structural proteins (e.g., capsid and/or envelope glycoproteins). For example, the self-replicating RNA is unable to produce RNA-containing alphavirus virions (i.e., infectious viral particles).

In one example, the self-replicating RNA comprises a native alphavirus SG promoter. For example, the native alphavirus SG promoter is a minimal SG promoter (i.e., the minimal sequence required for initiation of transcription) and comprises a sequence set forth in SEQ ID NO: 32.

The skilled person will be aware of alphaviruses suitable for use in the present disclosure. Exemplary alphaviruses include, but are not limited to, Venezuelan equine encephalitis virus (VEE; e.g., Trinidad donkey, TC83CR), Semliki Forest virus (SFV), Sindbis virus (SIN), Ross River virus, Western equine encephalitis virus, Eastern equine encephalitis virus, Chikungunya virus, S.A. AR86 virus, Everglades virus, Mucambo virus, Barmah Forest virus, Middelburg virus, Pixuna virus, O'nyong-nyong virus, Getah virus, Sagiyama virus, Bebaru virus, Mayaro virus, Una virus, Aura virus, Whataroa virus, Banbanki virus, Kyzylagach virus, Highlands J virus, Fort Morgan virus, Ndumu virus, and Buggy Creek virus. The term alphavirus may also include chimeric alphaviruses (e.g., as described by Perri et al, (2003) J. Virol. 77(19): 10394-403) that contain genome sequences from more than one alphavirus.

### Regulatory Elements

The present disclosure provides a monocistronic mRNA (e.g., a cRNA or self-replicating RNA) comprising a nucleotide sequence encoding a chemoattractant or an antigen. In an example, the present disclosure provides a monocistronic self-replicating RNA comprising a nucleotide sequence encoding a chemoattractant or an antigen operably linked to a regulatory element selected from the group consisting of a Kozak consensus sequence, an internal ribosome entry site (IRES), or a subgenomic (SG) promoter.

In one example, the present disclosure provides a monocistronic self-replicating RNA comprising a nucleotide sequence encoding a chemoattractant, or a nucleotide sequence encoding an antigen, operably linked to a SG promoter.

The present disclosure also provides a multicistronic mRNA (e.g., a cRNA or self-replicating RNA) comprising a first nucleotide sequence encoding a chemoattractant and a second nucleotide sequence encoding an antigen of interest. In one example, the nucleotide sequences encoding the chemoattractant or antigen of interest are operably linked to a regulatory element selected from the group consisting of a Kozak consensus sequence, an IRES, a SG promoter and combinations thereof.

In one example, the first nucleotide sequence encoding a chemoattractant and the second nucleotide sequence encoding an antigen of interest are operably linked to a regulatory element selected from the group consisting of a SG promoter and an IRES.

In one example, the present disclosure provides a multicistronic self-replicating RNA comprising a first nucleotide sequence encoding a chemoattractant operably linked to a SG promoter; and a second nucleotide sequence encoding an antigen operably linked to a regulatory element a promoter selected from the group consisting of a SG promoter and an IRES.

### Subgenomic Promoter

SG promoters (also known as ‘junction region’ promoters) suitable for use in the present disclosure will be apparent to the skilled person and/or are described herein.

In one example, the SG promoter is derived from or based on an alphavirus SG promoter. For example, the SG promoter is a native alphavirus SG promoter. In one example, the native SG promoter is a minimal SG promoter. For example, the minimal SG promoter is the minimal sequence required for initiation of transcription. In one example, the native SG promoter is an extended SG promoter. For example, the extended SG promoter is a minimal SG promoter extended at the 5′ end with nucleotides occurring in a sequence encoding a non-structural protein (e.g., NSP4) of the RNA virus (e.g., an alphavirus). In one example, the extended SG promoter is a minimal SG promoter extended at the 5′ end with nucleotides occurring in a sequence encoding an alphavirus NSP4.

In one example, the polynucleotide of the disclosure comprises a SG promoter from any alphavirus. For example, the RNA of the disclosure (e.g., cRNA or self-replicating RNA) comprises a SG promoter from any alphavirus.

In one example, the self-replicating RNA comprises a SG promoter from any alphavirus.

In one example, the polynucleotide of the present disclosure is a multicistronic mRNA that comprises two or more nucleotide sequences encoding a chemoattractant and an antigen of interest. In one example, the two or more nucleotide sequences are each operably linked to SG promoters. When two or more SG promoters are present in the RNA of the present disclosure, the promoters can be the same or different. For example, the two or more SG promoters are derived from the same alphavirus. In another example, the two or more SG promoters are derived from different alphaviruses.

When two or more SG promoters are present in the self-replicating RNA of the present disclosure, the promoters can be the same or different. For example, the two or more SG promoters are derived from the same alphavirus.

In another example, the two or more SG promoters are derived from different alphaviruses.

Exemplary SG promoter sequences are encoded by the sequences in SEQ ID NOs: 32, 33, 34, 36, 46, 51 and 52.

### Internal Ribosomal Entry Site (IRES)

IRES sequences suitable for use in accordance with the present disclosure will be apparent to the skilled person and/or are described herein.

In one example, the IRES is derived from encephalomyocarditis virus (EMCV). For example, the IRES is a wild-type IRES from EMCV.

In one example, the IRES is derived from a fibroblast growth factor 1A (FGF1A) IRES.

In addition, synthetic IRES elements have been described, which can be designed, according to methods know in the art to mimic the function of naturally occurring IRES elements (see Chappell, S A et al. Proc. Natl Acad. Sci. USA (2000) 97(4): 1536-41).

### Kozak Consensus Sequence

As used herein, the term “Kozak consensus sequence” refers to a nucleotide sequence identified in eukaryotic genes that facilitates the translation of the gene by containing a start codon (also referred to as a translation initiation codon) which is recognised by a ribosome.

Exemplary Kozak consensus sequence are known in the art and/or described herein. In one example, the Kozak consensus sequence is GCCACC. In one example, the Kozak consensus sequence is ACCATGG. In another example, the Kozak consensus sequence is ACCATG.

### 5′ Untranslated Region (5′UTR)

In one example, an RNA of the present disclosure comprises a 5′-UTR, such as the 5′ UTR of an RNA virus. In one example, the self-replicating RNA comprises a 5′-UTR of an RNA virus.

As used herein, the term “5′-untranslated region” or “5′-UTR” refers to a non-coding region of an mRNA located at the 5′end of the translation initiation sequence (AUG).

Exemplary 5′-UTRs include, for example, 5′-UTR of haptoglobin (HP), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

In one example, the 5′UTR is a 5′UTR of a Venezuelan equine encephalitis virus (VEEV) or modified forms thereof. For example, the 5′UTR comprises a sequence set forth in SEQ ID NO: 44.

In one example, the 5′UTR comprises at least one microRNA binding site, an AU rich element (ARE), a GC-rich element, a stem loop, and combinations thereof.

microRNA Binding Site

As used herein, the term “microRNA binding site” refers to a sequence within a polynucleotide (e.g. within a DNA or RNA transcript) that has sufficient complementarity to all or one region of a miRNA to interact, associate or bind to the microRNA (miRNA).

As used herein, the term “microRNA” or “miRNA” refers to 19-25 nucleotide long non-coding RNAs that bind to the 5′-UTR of polynucleotides and down-regulate gene expression (e.g. by inhibiting translation). The presence of microRNA binding site(s) in the 5′UTR of the present disclosure can function to inhibit translation of the 5′-UTR.

Suitable miRNA binding sites for use in the present disclosure will be apparent to the skilled person and/or described herein.

In one example, the miRNA binding site comprises a binding site for tissue specific microRNA or those regulating biological processes. For example, miRNA of the liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-id, miR-149), kidney (miR-192, miR-194, miR-204), and lung epithelial cells (let-7, miR-133, miR-126). For example, microRNA that regulate biological processes such as angiogenesis (miR-132). Further exemplifying miRNA and miRNA binding sites are disclosed in US patent application U.S. Ser. No. 14/043,927.

### AU Rich Element (ARE)

As used herein, the term “AU rich element (ARE)” or “AU rich elements (AREs)” refers to a region of a nucleotide sequence comprising stretches of Adeonisine (A) and Uridine (U). Exemplary AREs include, for example, ARE from cytoplasmic myc (c-myc), myoblast determination protein 1 (myoD), c-Jun, Myogenin, granulocyte-macrophage colony-stimulating factor (GM-CSF) and tumour necrosis factor alpha (TNF-α), or a combination thereof.

In one example, the ARE comprises a human antigen R or “HuR” (also known as Elavl1) specific binding site. HuR is known to bind AREs increasing the stability of the mRNA.

### GC-Rich Element

As used herein, the term “GC-rich element” refers to a nucleotide sequence with a high amount of Guanine (G) and/or Cytosine (C) compared to Adenine (A) and Thymine(T)/Uracil(U). The presence of GC-rich elements in a polynucleotide (e.g. mRNA) can stabilise the mRNA.

In one example, the GC-rich element comprises a sequence of 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25, or 26, or 27, or 28, or 29, or 30 nuceleotides in length.

In one example, the GC-rich element comprises between 30% and 40%, or 40% and 50%, or 50% and 60%, or 60% and 70% cytosine. For example, the GC-rich element comprises between 30% and 40% cytosine. For example, the GC-rich element comprises between 40% and 50% cytosine. For example, the GC-rich element comprises between 50% and 60% cytosine. For example, the GC-rich element comprises between 60% and 70% cytosine.

In one example, the GC-rich element comprises 30%, or 40%, or 50%, or 60%, or 70% cytosine. For example, the GC-rich element comprise 30% cytosine. For example, the GC-rich element comprises 40% cytosine. For example, the GC-rich element comprises 50% cytosine. For example, the GC-rich element comprises 60% cytosine. For example, the GC-rich element comprises 60% cytosine. For example, the GC-rich element comprises 70% cytosine.

In one example, the GC-rich element is at least 50% cytosine.

In one example, the GC-rich element is at least 60% cytosine.

In one example, the GC-rich element is at least 70% cytosine.

In one example, the GC-rich element comprises a nucleotide sequence CCCCGGCGCC (SEQ ID NO: 41). In another example, the GC-rich element comprises a nucleotide sequence CCCCGGC. In a further example, the GC-rich element comprises a nucleotide sequence GCGCCCCGCGGCGCCCCGCG (SEQ ID NO: 53).

In one example, the GC-rich element comprises a nucleotide sequence set forth in SEQ ID NO: 40 or 41. In one example, the GC-rich element comprises a nucleotide sequence set forth in SEQ ID NO: 40. In another example, the GC-rich element comprises a nucleotide sequence set forth in SEQ ID NO: 41. In a further example, the GC-rich element comprises a nucleotide sequence of CCCCGGC.

### Stem Loop

As used herein, the term “stem loop” refers to a nucleotide sequence comprising an intramolecular base pairing of two neighboured entirely or partially reverse complementary sequences to form a stem-loop. A stem-loop can occur in single-stranded DNA or, more commonly, in RNA. The stem loop can also be referred to as a hairpin or hairpin loop which usually consists of a stem and a terminal loop within a consecutive sequence, wherein the stem is formed by two neighboured entirely or partially reverse complementary sequences separated by a short sequence which builds the loop into a stem-loop structure.

The stability of the paired stem loop is determined by the length, the number of mismatched or bulges it contains, and the nucleotide composition of the paired region.

In one example, a loop of the stem loop is between 3 and 10 nucleotides in length. For example, the loop of the stem loop is between 3 and 8, or 3 and 7, or 3 and 6, or 4 and 5 nucleotides in length.

In one example, the loop of the stem loop is 4 nucleotides in length.

In one example, the stem loop is a histone stem loop. For example, the histone stem loop comprises or consist of a nucleotide sequence set for in SEQ ID NO: 43.

### 3′Untranslated Region (3′-UTR)

In one example, an RNA of the present disclosure comprises a 3′-untranslated region (3′-UTR).

As used herein, the term “3′-UTR” refers to a region of an mRNA located at the 3′end of the translation termination codon (i.e. stop codon).

Exemplary 3′-UTRs include, for example, a 3′-UTR of arachidonate 5-lipoxygenase (ALOX5), alpha I collagen (COL1A1), tyrosine hydroxylase (TH) gene, amino-terminal enhancer of split (AES), human mitochondrial 12S rRNA (mtRNR1), a fragment and/or a variant thereof.

In one example, the 3′UTR is a 3′UTR of a Sindbis virus (SINV) or modified forms thereof. For example, the 3′UTR comprises a sequence set forth in SEQ ID NO: 45.

In one example, the 3′-UTR comprises or consists of a nucleotide sequence derived from a 3′-UTR of an albumin gene. In one example, the 3′-UTR comprises or consists of a nucleotide sequence derived from a 3′-UTR of a vertebrate α-globin gene. For example, the 3′-UTR comprises or consists of a nucleotide sequence derived from a 3′-UTR of a mammalian α-globin gene. For example, the 3′-UTR comprises or consists of a nucleotide sequence derived from a 3′-UTR of a human α-globin gene.

In one example, the 3′-UTR of the present disclosure further comprises at least one microRNA binding site, an AU rich element (ARE), a GC-rich element, a triple helix, a stem loop, one or more stop codons or a combination thereof.

### Stop Codon

As used herein, the term “stop codon” refers to a trinucleotide sequence within a mRNA that signals the stop of protein synthesis by a ribosome.

In one example, the polynucleotide of the present disclosure comprises at least one stop codon at the 5′end of a 3′-UTR. For example, the stop codon is selected from UAG, UAA, and UGA.

In one example, the polynucleotide comprises two consecutive stop codons comprising a sequence UGAUGA.

In one example, the polynucleotide comprises two consecutive stop codons comprising a sequence UAAUAG.

### 3′ Tailing Sequence

The RNA of the present disclosure may comprise one or more 3′ tailing sequences located at the 3′end of the 3′UTR.

As described herein, the term “3′ tailing sequence” or “3′ tailing sequences” refers to a nucleotide sequence (e.g. polyadenylation signal) which induces the addition of non-encoded nucleotides to the 3′end of a mRNA or a nucleotide sequence (e.g. poly-A sequence) located at the 3′ end of a mRNA. A skilled person will appreciate that the 3′tailing sequence and/or products of the 3′tailing sequence in a mRNA functions to stabilise the mRNA and/or prevent the mRNA from degradation.

As used herein, the term “interrupting linker” in reference to a poly-A or poly-C sequence of the present disclosure refers to a single nucleotide or nucleotide sequence which are linked to, and interrupt, a stretch of consecutive adenosine or cytosine nucleotides in the poly-A or poly-C sequence. For example, the interrupting linker in a poly-A sequence is a single nucleotide or a nucleotide sequence consisting or comprising a nucleotide other than an adenosine nucleotide. For example, the interrupting linker in a poly-C sequence is a single nucleotide or a nucleotide sequence consisting or comprising a nucleotide other than a cytosine nucleotide.

In one example, the one or more 3′ tailing sequences are selected from the group consisting of a poly-A sequence, polyadenylation signal, a G-quadruplex, a poly-C sequence, a stem loop and combinations thereof.

### Poly-A Sequence

As used herein, the term “polyA sequence” refers to a nucleotide sequence of Adenine (A) located at the 3′end of a mRNA. In the context of the present disclosure, the polyA sequence may be located within the mRNA or DNA (e.g. a DNA plasmid serving as a template for generating the mRNA by transcription of the vector).

Suitable poly-A sequence for use in the present disclosure will be apparent to the skilled person and/or are described herein. In one example, the poly-A sequence comprises consecutive (i.e. one after the other) adenosine nucleotides of any length (e.g. to 10 to 300). For example, the poly-A sequence comprises 36 consecutive adenosine nucleotides. In one example, the poly-A sequence comprises a sequence set forth in SEQ ID NO: 47.

In one example, the poly-A sequence comprises consecutive adenosine nucleotides separated by one or more interrupting linkers. In one example, the poly-A sequence comprises consecutive adenosine nucleotides without an interrupting linker.

### Polyadenylation Signal

As used herein, the term “polyadenylation signal” refers to a nucleotide sequence which induces polyadenylation. Polyadenylation is typically understood to be the addition of a polyA sequence to a RNA (e.g. to a premature mRNA to generate a mature mRNA). The polyadenylation signal may be located within a nucleotide sequence at the 3′-end of the polynucleotide (e.g. mRNA) to be polyadenylated.

Suitable polyadenylation signal for use in the present disclosure will be apparent to the skilled person and/or described herein.

In one example, the polyadenylation signal comprises a hexamer consisting of Adenine and Uracil/Thymidine nucleotides. In one example, the hexamer sequence comprises or consists of AAUAAA.

In one example, the 3′tailing sequence comprises a polyadenylation signal but does not comprise a polyA sequence.

### G-Quadruplex

As used herein, the term “G-quadruplex” or “G4” refers to a nucleotide sequence rich in guanine residues which forms a four stranded secondary structure. For example, the G-quadruplex is a cyclic hydrogen bonded array of four guanine nucleotides formed by G-rich sequences in both DNA and RNA.

In one example, the 3′ tailing sequence comprises a polyA sequence and a G-quadruplex. For example, the 3′ tailing sequence comprises a polyA sequence linked to a G-quadruplex to produce a polyA-G quartet.

### Poly-C Sequence

As used herein, the term “poly-C sequence” refers to a nucleotide sequence of Cytosine (C) located at the 3′end of a mRNA. In the context of the present disclosure, the polyC sequence may be located within the mRNA or DNA (e.g. a DNA plasmid serving as a template for generating the mRNA by transcription of the vector).

Suitable poly-C sequence for use in the present disclosure will be apparent to the skilled person and/or are described herein.

In one example, the one or more 3′ tailing sequences comprises one or more poly-C sequences each comprising between 10 and 300 consecutive cytosine nucleotides. For example, the one or more poly-C sequences each comprises between 10 and 20, or 20 and 30, or 30 and 40, or 40 and 50, or 50 and 60, or 60 and 70, or 70 and 80, or 80 and 90, or 90 and 100, or 100 and 125, or 125 and 150, or 150 and 175, or 175 and 200, or 200 and 225, or 225 and 250, or 250 and 275, or 275 and 300 consecutive cytosine nucleotides. For example, the one or more poly-C sequence each comprises 10, or 20, or 30, or 40, or 50, or 60, or 70, or 80, or 90, or 100, or 125, or 150, or 175, or 200, or 225, or 250, or 275, or 300 consecutive cytosine nucleotides.

In one example, the one or more poly-C sequences is separated by an interrupting linker. For example, the fourth nucleotide sequence comprising the one or more 3′tailing sequences comprises, in order of 5′ to 3′: consecutive cytosine nucleotides, an interrupting linker, and further consecutive cytosine nucleotides.

In one example, the interrupting linker is from 10 to 50, or 50 to 100, or 100 to 150 nucleotides in length. For example, the interrupting linker is 1, or 2, or 3, or 4, or 5, or 6, or 7, or 8, or 9, or 10, or 11, or 12, or 13, or 14, or 15, or 16, or 17, or 18, or 19, or 20, or 25, or 30, or 35, or 40, or 45, or 50, or 55, or 60, or 65, or 70, or 75, or 80, or 85, or 90, or 95, or 100, or 110, or 120, or 130, or 140, or 150 nucleotides in length.

### 5′Cap

In one example, an mRNA according to the present disclosure comprises a 5′terminal cap structure.

As used herein, the term “5′cap structure” refers to a structure at the 5′ terminal end of a mRNA involved in nuclear export and binds a mRNA Cap Binding Protein (CBP). The 5′cap structure is known to stabilise mRNA through association of CBP with poly(A) binding protein to form a mature mRNA. Accordingly, the presence of a 5′cap structure in the mRNA of the present disclosure can further increase the stability of the mRNA compared to a mRNA without the 5′cap.

Exemplary 5′cap structure includes, for example, anti-reverse cap analogue (ARCA), N7,2′-0-dimethyl-guanosine (mCAP), inosine, N1-methyl-guanosine, 2′fluoro-guanosine, 7-deaza-guanosine, 8-oxo-guanosine, 2-amino-guanosine, LNA-guanosine, 2-azido-guanosine, N6,2′-O-dimethyladenosine, 7-methylguanosine (m7G), Cap1, and Cap2.

Typically, an endogenous mRNA is 5′capped with a guanosine through a (5)′-ppp-(5)′-triphosphate linkage attached to the 5′terminal nucleotide of the mRNA. The guanosine cap can then be methylated to a 7-methylguanosine (m7G) generating a 7mG(5′)ppp(5′)N, pN2p (Cap0 structure), where N represents the first and second 5′terminal nucleotide of the mRNA. The cap0 structure can be further 2′-O-methylated to produce 7mG(5′)ppp(5′)NlmpNp (Cap1), and/or 7mG(5′)-ppp(5′)NlmpN2mp (Cap2).

In one example, the polynucleotide of the present disclosure comprises an endogenous cap.

As used herein, the term “endogenous cap” refers to a 5′cap synthesised in a cell. For example, endogenous cap is a natural 5′cap or a wild-type 5′cap. For example, the endogenous cap is a Cap0, Cap1, or Cap2 structure.

In one example, the polynucleotide of the present disclosure comprises an analog of an endogenous cap (also referred to as cap analog).

As used herein, the term “analogue thereof” in the context of an endogenous cap or “cap analog” refers to a synthetic 5′cap. The cap analog can be used to produce 5′capped mRNA in in vitro transcription reactions. Cap analogs may be chemically (i.e. non-ezymatically) or enzymatically synthesized and/or linked to a nucleotide (e.g. 5′terminal nucleotide of an mRNA). Exemplary cap analogs are commercially available and include, for example, 3″-O-Me-m7G(5′)ppp(5′)G, G(5′)ppp(5′)A, G(5′)ppp(5′)G, m7G(5′)ppp(5′)A, m7G(5′)ppp(5′)G (New England BioLabs). In one example, the cap analog is N7,3′-O-dimethyl-guanosine-5′-triphosphate-5′-guanosine (i.e. anti-reverse cap analogue (ARCA)).

In one example, the 5′cap structure is a non-hydrolyzable cap structure. The non-hydrolyzable cap structure can prevent decapping of the mRNA and increase the half-life of the mRNA.

In one example, the non-hydrolyzable cap structure comprises a modified nucleotide selected from a group consisting or a α-thio-guanosine nucleotide, α-methyl-phosphonate, seleno-phosphate, and a combination thereof. In one example, the modified nucleotide is linked to the 5′end of the mRNA through an α-phosphorothiate linkage. Methods of linking the modified nucleotide to the 5′end of the mRNA will be apparent to the skilled person. For example, using a Vaccinia Capping Enzyme (New England Biolabs).

### Modifications

In one example, the polynucleotide of the present disclosure comprises one or more modification(s). Typically, modifications are introduced into a polynucleotide (e.g. mRNA) to increase the translation efficiency and/or stability of the polynucleotide. Suitable modifications to the polynucleotide will be apparent to the skilled person and/or described herein.

In one example, the first nucleotide sequence comprising the 5′-UTR and/or the fragment thereof is modified. Modification of the first nucleotide sequences comprising the 5′-UTR and/or the fragment thereof results in a variant of the 5′-UTR and/or the fragment thereof.

In one example, one or more nucleotide sequence(s) of the polynucleotide are codon optimized. Method of codon optimization will be apparent to the skilled person and/or described herein. For example, tools for codon optimization of polynucleotide include, for example, GeneArt GeneOptimizer (Thermofisher®) or GenSmart® (GeneScript®).

In one example, the polynucleotide is modified to increase the amount of Guanine (G) and/or Cytosine (C) in the polynucleotide. The amount of G/C in the polynucleotide (i.e. G/C content) can influence the stability of the polynucleotide. Accordingly, polynucleotide comprising an increased amount of G/C nucleotides can be functionally more stable than polynucleotides containing a large amount of Adenine (A) and Thymine (T) or Uracil (U) nucleotides. The G/C content is increased by substituting A or T nucleotides with G or C nucleotides.

In one example, the G/C content is increased in the first and/or second nucleotide sequence encoding the first and/or second antigen of interest. In one example, the G/C content is increased in the first and/or second nucleotide sequence encoding the first and/or second antigen of interest and/or the one or more additional nucleotide sequences encoding the one or more antigens of interest. The modification(s) in the first and/or second and/or one or more nucleotide sequences takes advantage of the ability of substituting codons that contain less favourable combinations of nucleotides (in terms of mRNA stability) with alternative codons encoding the same amino acid, or encoding amino acid(s) of similar chemistry (e.g. conserved amino acid substitution). For example, the G/C content is increased by substituting codons containing A or T nucleotides with codons containing G or C nucleotides that encode for the same amino acid. For example, the G/C content is increased by substituting codons containing A or T nucleotides with codons containing G or C nucleotides that encode for an amino acid of similar chemistry.

In one example, the G/C content is increased in one or more nucleotide sequences of the polynucleotide which do not encode the antigen of interest. For example, the G/C content is increased in the 5′-UTR, the fragment and/or the variant thereof. For example, the G/C content is increased in the 3′-UTR, the fragment and/or the variant thereof.

In one example, the polynucleotide comprises at least one chemically modified nucleotide.

As used herein, the term “chemical modification” or “chemical modified” in the context of a nucleotide refers to a naturally occurring nucleotides (i.e. A, T, C, G, U) which are modified by replacement, insertion or removal of individual or several atoms or atomic groups compared to the naturally occurring nucleotides. In one example, at least one naturally occurring nucleotide of the polynucleotide is replaced with a chemically modified nucleotide. In one example, at least 10%, or 20%, or 30%, or 40%, or 50%, or 60%, or 70%, or 80%, or 90%, or 100% of naturally occurring nucleotides of the polynucleotide is replaced with a chemically modified nucleotides. Suitable chemical modified nucleotides for use in the present disclosure will be apparent to the skilled person and/or described herein. Exemplary chemically modified nucleotides include, for example, N6,2′-0-dimethyl-adenosine (m6Am), 5-methyluridine (m5U), N4-acetylcytidine (ac4C), 2-thiocytidine (s2C), 2-thiouridine (s2U), 5-methylcytidine (m5C), N6-methyladenosine (m6a), pseudouridine (ψ), and 1-methylpseudouridine (m1ψ).

### Pharmaceutical Compositions

The present disclosure provides an immunogenic composition comprising a polynucleotide of the present disclosure.

The present disclosure provides an immunogenic composition comprising a RNA of the present disclosure.

The present disclosure also provides an immunogenic composition comprising a cRNA of the present disclosure.

The present disclosure further provides an immunogenic composition comprising a self-replicating RNA of the present disclosure.

The present disclosure also provides a pharmaceutical composition comprising an immunogenic composition of the present disclosure and a pharmaceutically acceptable carrier.

It will be apparent to the skilled person and/or described herein, that the polynucleotide, RNA, cRNA and/or self-replicating RNA of the present disclosure may be present as naked RNA or in combination with lipids, polymers or other delivery system that facilitates entry into the cells.

### Delivery Systems

In one example, the pharmaceutical composition of the present disclosure further comprises a LNP, a polymeric microparticle and an oil-in-water emulsion. For example, the polynucleotide, the cRNA and/or the self-replicating RNA is encapsulated in, bound to or adsorbed on a LNP, a polymeric microparticle, or an oil-in-water emulsion.

### Lipid Nanoparticles

In one example, the pharmaceutical composition of the present disclosure further comprises a LNP.

It will be apparent that the term “lipid nanoparticle” or “LNP” refers to any lipid composition, including, but not limited to, liposomes or vesicles, where an aqueous volume is encapsulated by amphipathic lipid bilayers (e.g., single; unilamellar or multiple; multilamellar) micelle-like lipid nanoparticles having a non-aqueous core and solid lipid nanoparticles, wherein solid lipid nanoparticles lack lipid bilayers.

Lipid nanoparticles suitable for use in the present disclosure will be apparent to the skilled person and/or are described herein. The lipids can have an anionic, cationic or zwitterionic hydrophilic head group.

In one example, the lipid nanoparticle comprises a PEG-lipid, a sterol structural lipid and/or a neutral lipid. In one example, the lipid nanoparticle further comprises a cationic lipid. In one example, the lipid nanoparticle does not comprise a cationic lipid.

In one example, the LNP comprises a PEG-lipid. For example, the PEG-lipid is selected from the group consisting of PEG-c-DMG, PEG-DMG, PEG-DLPE, PEG-DMPE, PEG-DPPC, a PEG-DSPE lipid and combinations thereof.

In one example, the LNP comprises a structural lipid. For example, the structural lipid is selected from the group consisting of cholesterol fecosterol, sitosterol, campesterol, stigmasterol, brassicasterol, ergosterol, tomatidine, tomatine, ursolic acid and alpha-tocopherol and combinations thereof.

In one example, the LNP comprises a neutral lipid. Exemplary phospholipids (anionic or zwitterionic) for use in the present disclosure include, for example, phosphatidylethanolamines, phosphatidylcholines, phosphatidylserines, and phosphatidylglycerols. For example, the neutral lipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine (DSPE), 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin and combinations thereof.

In one example, the LNP comprises a cationic lipid. Exemplary cationic lipids include, but are not limited to, dioleoyl trimethylammonium propane (DOTAP), I,2-distearyloxy-N,N-dimethyl-3-aminopropane (DSDMA), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), 2,5-bis((9z,12z)-octadeca-9,12,dien-1-yloxyl)benzyl-4-(dimethylamino)butnoate (LKY750). In one example, the phospholipid is 2,5-bis((9z,12z)-octadeca-9,12,dien-1-yloxyl)benzyl-4-(dimethylamino)butnoate (LKY750). Exemplary zwitterionic lipids include, but are not limited to, acyl zwitterionic lipids and ether zwitterionic lipids, such as dipalmitoylphosphatidylcholine (DPPC), dioleoylphosphatidylcholine (DOPC) and dodecylphosphocholine. The lipids can be saturated or unsaturated.

### Polymeric Microparticles

In one example, the pharmaceutical composition of the present disclosure further comprises a polymeric microparticle.

The skilled person will be aware that various polymers can form microparticles to encapsulate or adsorb the polynucleotide, the cRNA and/or the self-replicating RNA of the present disclosure. It will be apparent that use of a substantially non-toxic polymer means that particles are safe, and the use of a biodegradable polymer means that the particles can be metabolised after delivery to avoid long-term persistence. Useful polymers are also sterilisable, to assist in the preparation of pharmaceutical grade formulations.

Exemplary non-toxic and biodegradable polymers include, but are not limited to, poly(α-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

### Oil-In-Water Cationic Emulsions

In one example, the pharmaceutical composition of the present disclosure further comprises an oil-in-water cationic emulsion.

Suitable oils for use in an oil-in-water emulsion will be apparent to the skilled person and/or are described herein. For example, the emulsion comprises one or more oils derived, for example, from an animal (e.g., fish) or a vegetable source (e.g., nuts, seeds, grains). The skilled person will recognise that biocompatible and biodegradable oils are preferentially used. Exemplary animal oils (i.e., fish oils) include cod liver oil, shark liver oils, and whale oil. Exemplary vegetable oils include peanut oil, coconut oil, olive oil, soybean oil, jojoba oil, safflower oil, cottonseed oil, sunflower seed oil, sesame seed oil, corn oil.

In addition to the oil, the oil-in-water emulsion also comprises a cationic lipid to facilitate formation and stabilisation of the emulsion. Suitable cationic lipids will be apparent to the skilled person and/or are described herein. Exemplary cationic lipids include, but are not limited to, limited to: I, 2-dioleoyloxy-3-(trimethylammonio)propane (DOTAP), 3′-[N-(N′,N′-Dimethylaminoethane)-carbamoyl]Cholesterol (DC Cholesterol), dimethyldioctadecyl-ammonium (DDA), I,2-Dimyristoyl-3-Trimethyl-AmmoniumPropane (DMTAP), dipalmitoyl[C16:0]trimethyl ammonium propane (DPTAP) and distearoyltrimethylammonium propane (DSTAP).

In some examples, the oil-in-water emulsion also comprises a non-ionic surfactant and/or a zwitterionic surfactant. The skilled person will be aware of surfactants suitable for use in the present disclosure. Exemplary surfactants include, but are not limited to: the polyoxyethylene sorbitan esters surfactants (e.g., polysorbate 20 and polysorbate 80) and copolymers of ethylene oxide (EO), propylene oxide (PO), and/or butylene oxide (BO).

### Pharmaceutically Acceptable Carrier

Suitably, in compositions or methods for administration of the polynucleotides, RNA, cRNA and/or the self-replicating RNA of the disclosure to a subject, the polynucleotide, RNA, cRNA and/or the self-replicating RNA is combined with a pharmaceutically acceptable carrier as is understood in the art. Accordingly, one example of the present disclosure provides a composition (e.g., a pharmaceutical composition) comprising the polynucleotide, RNA, cRNA and/or the self-replicating RNA of the disclosure (and any delivery system) combined with a pharmaceutically acceptable carrier.

In general terms, by “carrier” is meant a solid or liquid filler, binder, diluent, encapsulating substance, emulsifier, wetting agent, solvent, suspending agent, coating or lubricant that may be safely administered to any subject, e.g., a human. Depending upon the particular route of administration, a variety of acceptable carriers, known in the art may be used, as for example described in Remington's Pharmaceutical Sciences (Mack Publishing Co. N.J. USA, 1991).

The polynucleotides, RNA, cRNA and/or the self-replicating RNA of the present disclosure are useful for parenteral, topical, oral, or local administration, intramuscular administration, aerosol administration, or transdermal administration, for prophylactic or for therapeutic treatment. In one example, the self-replicating RNA is administered parenterally, such as intramuscularly, subcutaneously or intravenously. For example, the polynucleotides, RNA, cRNA and/or the self-replicating RNA may be administered intramuscularly. In another example, the polynucleotides, RNA, cRNA and/or the self-replicating RNA is administered parenterally, such as intramuscularly, subcutaneously or intravenously. For example, the polynucleotides, RNA, cRNA and/or the self-replicating RNA is administered intramuscularly.

Formulation of a polynucleotide, RNA, cRNA and/or the self-replicating RNA to be administered will vary according to the route of administration and formulation (e.g., solution, emulsion, capsule) selected. An appropriate pharmaceutical composition comprising a polynucleotide, RNA, cRNA and/or the self-replicating RNA to be administered can be prepared in a physiologically acceptable carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. A variety of appropriate aqueous carriers are known to the skilled artisan, including water, buffered water, buffered saline, polyols (e.g., glycerol, propylene glycol, liquid polyethylene glycol), dextrose solution and glycine. Intravenous vehicles can include various additives, preservatives, or fluid, nutrient or electrolyte replenishers (See, generally, Remington's Pharmaceutical Science, 16th Edition, Mack, Ed. 1980). The compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. The polynucleotides, RNA, cRNA and/or the self-replicating RNA can be stored in the liquid stage or can be lyophilized for storage and reconstituted in a suitable carrier prior to use according to art-known lyophilization and reconstitution techniques.

The optimum concentration of the active ingredient(s) in the chosen medium can be determined empirically, according to procedures known to the skilled artisan, and will depend on the ultimate pharmaceutical formulation desired.

Upon formulation, compositions of the present disclosure will be administered in a manner compatible with the dosage formulation and in such amount as is therapeutically/prophylactically effective. The dosage ranges for the administration of the polynucleotides, RNA, cRNA and/or the self-replicating RNA of the disclosure are those large enough to produce the desired effect. For example, the composition comprises an effective amount of the polynucleotides, RNA, cRNA and/or the self-replicating RNA. In one example, the composition comprises a therapeutically effective amount of the self-replicating RNA. In another example, the composition comprises a prophylactically effective amount of the self-replicating RNA. In one example, the composition comprises an effective amount of the RNA. In one example, the composition comprises a therapeutically effective amount of the RNA. In another example, the composition comprises a prophylactically effective amount of the RNA.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the polynucleotide, RNA, cRNA and the self-replicating RNA is administered at an initial (or loading) dose which is higher than subsequent (maintenance doses). For example, the polynucleotide, RNA, cRNA and/or the self-replicating RNA is administered at an initial dose of between about 10 mg/kg to about 30 mg/kg. The polynucleotide, RNA, cRNA or the self-replicating RNA is then administered at a maintenance dose of between about 0.0001 mg/kg to about 10 mg/kg. The maintenance doses may be administered every 7-35 days, such as, every 7 or 14 or 28 days.

In some examples, a dose escalation regime is used, in which the polynucleotide, RNA, cRNA or the self-replicating RNA is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the polynucleotide, RNA, cRNA or the self-replicating RNA of the present disclosure. A subject may be retreated with the polynucleotide, RNA, cRNA or the self-replicating RNA by being given more than one exposure or set of doses, such as at least about two exposures of the binding protein, for example, from about 2 to 60 exposures, and more particularly about 2 to 40 exposures, most particularly, about 2 to 20 exposures.

In one example, any retreatment may be given when signs or symptoms of disease return.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

In the case of a subject that is not adequately responding to treatment, multiple doses in a week may be administered. Alternatively, or in addition, increasing doses may be administered.

In another example, for subjects experiencing an adverse reaction, the initial (or loading) dose may be split over numerous days in one week or over numerous consecutive days.

Administration of the polynucleotide, RNA, cRNA or the self-replicating RNA according to the methods of the present disclosure can be continuous or intermittent, depending, for example, on the recipient's physiological condition, whether the purpose of the administration is therapeutic or prophylactic, and other factors known to skilled practitioners. The administration of the polynucleotide, RNA, cRNA or the self-replicating RNA may be essentially continuous over a preselected period of time or may be in a series of spaced doses, e.g., either during or after development of a condition.

### Adjuvants

Pharmaceutical compositions according to the present disclosure can comprise additional adjuvants or immunopotentiating agents. An adjuvant or immunopotentiating agent is a substance that modifies the action of the principal ingredient. Thus, a skilled person will understand that the terms “adjuvant” or “immunopotentiating agent” may be used interchangeably. In an example, an adjuvant or immunopotentiating agent is a substance that enhances a subject's immune response to an antigen. Suitable adjuvants or immunopotentiating agents will be appearent to the skilled person and include, for example, aluminium-containing adjuvants (e.g. amorphous aluminum hydroxyphosphate sulfate, aluminum hydroxide, aluminum phosphate, and potassium aluminum sulfate), AS04, MF59, AS01B, or CpG 1018.

### Methods of Production

Suitable methods for the production of polynucleotides, RNA, cRNA or the self-replicating RNA according to the present disclosure will be apparent to the skilled person and/or described herein.

In one example, the cRNA is produced using a plasmid DNA. In one example, the self-replicating RNA is produced using a plasmid DNA. The skilled person will understand that plasmid DNA is relatively stable. Briefly, competent bacterial cells (e.g., Escherichia coli) cells are transformed with a DNA plasmid encoding a self-replicating RNA of the present disclosure. Individual bacterial colonies are isolated and the resultant plasmid DNA amplified in E. coli cultures.

In one example, the plasmid DNA suitable for use in accordance with the disclosure has a sequence according to SEQ ID NO: 50 or has a sequence that is at least 70%, at least 75%, at least 80%, at least 85%, at least 90%, at least 95% or at least 100% identical to SEQ ID NO: 50.

With reference to SEQ ID NO:50, base pairs 1 to 44 comprise the 5′UTR, base pairs 45 to 1649 comprise nsP1, base pairs 1650 to 4031 comprise nsP2, base pairs 4032 to 5681 comprise nsp3, base pairs 5682 to 7526 comprise nsp4, base pairs 7513 to 7536 comprise the subgenomic promoter, base pairs 7562 to 9253 comprise the Turkey HA antigen, base pairs 9276 to 9594 comprise the 3′UTR, base pairs 9823 to 10490 comprise ColE1, base pairs 10634 to 11449 comprise a kanamycin resistance transposon and base pairs 11867 to 11883 comprise the T7 promoter.

In one example, the plasmid DNA is isolated following fermentation. For example, the plasmid DNA is isolated using a commercially available kit (e.g., Maxiprep DNA kit), or other routine methods known to the skilled person. Following isolation, plasmid DNA is linearized by restriction digest (i.e., using a restricting enzyme). Restriction enzymes are removed using methods known in the art, including for example phenol/chloroform extraction and ethanol precipitation.

In one example, mRNA is made by in vitro transcription from a linearized DNA template using an RNA polymerase (e.g., T7 RNA polymerase). Following in vitro transcription, the DNA template is removed by DNase digestion. The skilled person will understand that synthetic mRNA capping is performed to correct mRNA processing and contribute to stabilization of the mRNA. In one example, the mRNA is enzymatically 5′-capped. For example, the 5′ cap is a cap0 structure or a cap1 structure. In one example, the 5′ cap is a cap0 structure, for example, the 5′-cap (i.e., cap0) consists of an inverted 7-methylguanosine connected to the rest of the mRNA via a 5′-5′ triphosphate bridge. In one example, the 5′ cap is a cap1 structure, for example, the 5′-cap (i.e., cap1) consists of the cap0 with an additional methylation of the 2′O position of the initiating nucleotide.

In one example, the mRNA is purified. Various methods for purifying mRNA will be apparent to the skilled person. For example, the mRNA is purified using lithium chloride (LiCl) precipitation. In another example, the mRNA is purified using tangential flow filtration (TFF). Following purification, the mRNA is resuspended in e.g., nuclease-free water.

### Screening Assays

Suitable methods for selecting a polynucleotide, RNA, cRNA or the self-replicating RNA of the present disclosure are available to those skilled in the art. Assays may be conducted to assess the efficiency and efficacy of the RNA including, for example, serology and immune responses.

### Polypeptide Expression

In one example, the self-replicating RNA is assessed for expression of the antigen and chemoattractant of interest.

For example, antigen expression is detected using antibodies against the antigen of interest. In one example, the number of cells positive for antigen expression is measured by e.g., fluorescence-activated cell sorting (FACS). In another example the mean fluorescence intensity (MFI) is determined using e.g., FACS. In a further example, the specific potency value or the probability of successful transfection per unit mass of RNA is calculated.

### Microneutralization Assay

In one example, the self-replicating RNA (naked and/or formulated) is assessed for antibody responses. For example, the self-replicating RNA is assessed using a microneutralisation assay. Methods of performing a microneutralization assay will be apparent to the skilled person. In one example, the microneutralization assay is a short form assay. For one example, a virus fluorescent focus-based microneutralization assay is performed. In another example, the microneutralization assay is a long form assay.

### Hemagglutination Inhibition (HAI) Assay

In one example, the self-replicating RNA (naked and/or formulated) is assessed for antibody responses. For example, the self-replicating RNA is assessed using a hemagglutination inhibition (HAI) assay. Methods of performing a HAI assay will be apparent to the skilled person and/or described, for example, in WHO (2011) Manual for the laboratory diagnosis and virological surveillance of influenza: WHO Press, World Health Organization.

### Antigen Specific T Cell Responses

In one example, the self-replicating RNA is assessed for its ability to induce antigen specific T cell responses. Methods of assessing induction of antigen specific T cell responses will be apparent to the skilled person and/or are described herein.

For example, antigen-specific T cell detection is performed on splenic cultures. Briefly, splenocyte cultures are established in T cell medium and cell cultures are either stimulated with antigenic peptides or unstimulated. In one example, antigen-specific T cell responses are determined using flow cytometry.

### Neutralising Assays

The self-replicating RNA of the disclosure may be screened in vitro for their ability to bind to an antigen e.g., SARS-CoV-2 S protein RBD and neutralise binding of the S protein RBD to ACE2. Suitable assays will be apparent to the skilled person and include, for example, a Vero microneutralisation assay, a sVNT assay, or a psuedovirus neutralisation assay (using e.g., HEK-293T cells or HeLa-ACE2 cells).

In one example, the neutralization assay is a Vero microneutralization assay. Briefly, SARS-Cov-2 wild-type virus is passaged in Vero cells (i.e., the Vero lineage isolated from kidney epithelial cells extracted from an African green monkey). Serial two-fold dilutions of a test protein are incubated with 100 TCID₅₀ (i.e., median tissue culture infectious dose) of SARS-CoV-2 for 1 hour and residual virus infectivity is assessed in Vero cells; viral cytopathic effect is read, for example, on day 5. The neutralising antibody titre is calculated using the Reed/Muench method as previously described (Houser et al., 2016; Subbarao et al 2004).

In one example, the neutralization assay is a surrogate neutralization test (sVNT). Briefly, the wells of a plate are coated with hACE2 protein in carbonate-bicarbonate coating buffer (e.g., pH 9.6). HRP-conjugated SARS-CoV-2 and HRP-conjugated SARS-CoV-RBD pre-incubated with test proteins is added to the hACE2 at different concentrations and incubated, for example, for 1 h at room temperature. Unbound HRP conjugated antigens are removed by washing. Colorimetric signal is developed on the enzymatic reaction of HRP with chromogenic substrate, e.g., 3,3′,5,5′-tetramethylbenzidine (TMB). In one example, the absorbance reading at 450 nm and 570 nm is acquired.

In one example, the neutralisation is a psuedovirus neutralisation assay. For example, HIV reporter virus pseudotyped with SARS-2-Spike protein is produced by co-transfection of SARS-2-COV-2 spike plasmids together with a viral backbone plasmid (e.g., pDR-NL Δenv FLUC) into e.g., HEK-293T cells. Pseudovirus is harvested post transfection and clarified by filtration. Virus stock titres, reported as Relative Luciferase Units infectious dose (RLU), are calculated by limiting dilution infections in Hela-hACE2 cells measuring luciferase activity as a read-out for viral infection.

### Methods of Treatment or Prevention

The present disclosure provides methods of using the immunogenic composition or the pharmaceutical composition of the present disclosure as a vaccine.

The present disclosure also provides methods of treating or preventing a disease or condition in a subject comprising administering the immunogenic composition or the pharmaceutical composition of the present disclosure. For example, the disease or condition is a respiratory virus infection, such as influenza, COVID-19, or respiratory syncytial virus (RSV). In one example, the disease or condition is acute respiratory distress syndrome (ARDS).

### Influenza

Influenza, also known as “the flu”, is an infectious disease caused by an influenza virus. Symptoms can be mild to severe and the most common symptoms include high fever, runny nose, sore throat, muscle and joint pain, headache, coughing, and feeling tired. Symptoms typically begin two days after exposure to the virus and most last less than a week. Complications of influenza may include viral pneumonia, secondary bacterial pneumonia, sinus infections, and worsening of previous health problems such as asthma or heart failure. Viral pneumonia may also lead to acute respiratory distress syndrome (ARDS).

It will be apparent to the skilled person that there are currently four influenza viruses—A, B, C and D. Influenza A virus is the most common flu virus infecting humans, animals, and birds, whilst influenza B virus infection mostly occurs in humans. Infection of influenza C virus does not cause any severe symptom in human or mammals and influenza D, to date, has only infected pigs and cattle.

Thus, in some examples of the present disclosure, the subject has an influenza virus infection. In one example, the subject has influenza. In particular, the influenza is associated with ARDS. In one example, the methods of the present disclosure can be used to treat or prevent ARDS in a subject suffering from an influenza virus infection. In one example, the methods of the present disclosure can be used to treat or prevent ARDS in a subject suffering from influenza.

In an example, the methods described herein comprise the identification of a subject having or suspected of having influenza. In this example, the subject may have one or more of the above symptoms and may be classified as having mild or severe influenza.

### Coronavirus Disease 2019 (COVID-19)

The present disclosure provides, for example, methods of treating or preventing COVID-19.

The present disclosure also provides, for example, methods of treating or preventing SARS-CoV-2 infection. In some examples of the present disclosure the subject has a SARS-CoV-2 infection but does not have clinically diagnosed COVID-19.

COVID-19 is an infectious disease caused by SARS-CoV-2. It was first identified in December 2019 in Wuhan, Hubei, China, and has resulted in an ongoing pandemic. Common symptoms include fever, cough, fatigue, shortness of breath, and loss of smell and taste. While the majority of cases result in mild symptoms, some progress to ARDS. The time from exposure to onset of symptoms is typically around five days, but may range from two to fourteen days. There are currently no vaccines nor specific antiviral treatments for COVID-19 and management involves the treatment of symptoms, supportive care, isolation, and experimental measures.

Thus, in some examples, the subject has a SARS-CoV-2 infection. In one example, the subject has COVID-19, for example, severe COVID-19. In particular, severe COVID-19 often results in ARDS. The methods of the present disclosure can be used to treat or prevent ARDS in a subject suffering from severe COVID-19.

In an example, the methods described herein comprise the identification of a subject having or suspected of having SARS-CoV-2. In this example, the subject may have one or more of the above symptoms and may be classified as having mild or severe SARS-CoV-2.

### Acute Respiratory Distress Syndrome (ARDS)

The present disclosure provides, for example, methods of treating or preventing ARDS in a subject.

ARDS is a life-threatening condition characterized by bilateral pulmonary infiltrates, severe hypoxemia, and disruption of the alveolar-capillary membrane barrier (i.e., pulmonary vascular leak), leading to non-cardiogenic pulmonary edema. There is currently no effective pharmacological therapy.

Infectious etiologies, including influenza and coronavirus infection, are leading causes of ARDS. Accordingly, in one example of the present disclosure, the ARDS is associated with an influenza or a coronavirus infection. For example, the ARDS is associated with influenza. In another example, the ARDS is associated with a coronavirus infection, such as a SARS-COV infection. In one example, the ARDS is associated with a SARS-CoV-2 infection.

ARDS is classified according to the Berlin Definition, which includes:
(1) presentation within 1 week of clinical insult or onset of respiratory symptoms;
(2)acute hypoxemic respiratory failure, as determined by a PaO2/FiO2 ratio of 300 mmHg or less on at least 5 cm of continuous positive airway pressure (CPAP) or positive end expiratory pressure (PEEP), where PaO2 is the partial pressure of oxygen in arterial blood and the FiO2 is the fraction of inspired oxygen;
(3) bilateral opacities on lung radiographs not fully explained by effusions, consolidation, or atelectasis; and
(4)edema/respiratory failure not fully explained by cardiac failure or fluid overload.

In one example, the subject has or suffers from ARDS (i.e., the subject satisfies the Berlin definition of ARDS). For example, the subject is in need of treatment (i.e., in need thereof).

In one example, the subject has or suffers from a symptom associated with ARDS. Symptoms associated with ARDS and methods of identifying subjects at risk of developing ARDS will be apparent to the skilled person and/or are described herein. For example, the subject has one or more or all of the following symptoms:
a) a respiratory frequency of greater than 30 breaths per minute;
b) an oxygen saturation (SpO₂) of 93% or less on room air;
c) a ratio of arterial partial pressure of oxygen to fraction of inspired oxygen (PaO₂/FiO₂) of less than 300 mmHg;
d) a SpO₂/FiO₂ ratio of less than 218; and
e) radiographic lung infiltrates in an amount of greater than 50%.

Currently, ARDS is classified as mild, moderate or severe with an associated increased mortality. The severity of ARDS can be categorized according to the Berlin definition as follows:
(i) Mild ARDS: PaO₂/FiO₂ of 200-300 mmHg on at least 5 cm CPAP or PEEP;
(ii) Moderate ARDS: PaO₂/FiO₂ of 100-200 mmHg on at least 5 cm PEEP; and
(iii) Severe ARDS: PaO₂/FiO₂ of less than or equal to 100 mmHg on at least 5 cm PEEP.

In one example, the ARDS is mild ARDS. In another example, the ARDS is moderate ARDS. In a further example, the ARDS is severe ARDS.

In an example, the methods described herein comprise the identification of a subject having or suspected of having ARDS. In this example, the subject may have one or more of the above symptoms and may be classified as having mild or severe ARDS.

The methods of the present disclosure can, in addition to treatment of existing ARDS, be used to prevent the onset of ARDS. Thus, in one example, the subject does not have ARDS.

### Respiratory Syncytial Virus (RSV)

The present disclosure provides, for example, methods of treating or preventing RSV.

In one example, the subject has or suffers from a symptom associated with RSV. Symptoms associated with RSV and methods of identifying subjects at risk of developing RSV will be apparent to the skilled person and/or are described herein. For example, the subject has one or more or all of the following symptoms indicative of mild RSV:
a) congested or runny nose;
b) dry cough;
c) low-grade fever;
d) sore throat;
e) sneezing;
f) headache;
   or in severe cases:
a) short, shallow and rapid breathing;
b) struggling to breathe—chest muscles and skin pull inward with each breath;
c) cough;
d) poor feeding;
e) unusual tiredness (lethargy);
f) irritability.

Thus, in one example, the RSV is mild RSV. In a further example, the RSV is severe RSV.

In an example, the methods described herein comprise the identification of a subject having or suspected of having RSV. In this example, the subject may have one or more of the above symptoms and may be classified as having mild or severe RSV.

The methods of the present disclosure can, in addition to treatment of existing RSV, be used to prevent the onset of RSV. Thus, in one example, the subject does not have RSV.

### Kits

Another example of the disclosure provides kits containing a self-replicating RNA of the present disclosure useful for the treatment or prevention of a disease or disorder as described above.

Another example of the disclosure provides kits containing a self-replicating RNA of the present disclosure useful for the treatment or prevention of a disease or disorder as described above.

In one example, the kit comprises (a) a container comprising a self-replicating RNA optionally in a delivery system and/or a pharmaceutically acceptable carrier or diluent; and (b) a package insert with instructions for treating or preventing a disease or disorder (e.g., influenza, COVID-19 or ARDS) in a subject.

In one example, the kit comprises (a) a container comprising a cRNA optionally in a delivery system and/or a pharmaceutically acceptable carrier or diluent; and (b) a package insert with instructions for treating or preventing a disease or disorder (e.g., influenza, COVID-19 or ARDS) in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the self-replicating RNA, RNA, cRNA or polynucleotide. The label or package insert indicates that the composition is used for treating a subject eligible for treatment, e.g., one having or predisposed to developing influenza, an influenza virus infection, a SARS-CoV-2 infection, COVID-19 and/or ARDS, with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

### EXAMPLES

### Example 1: Generation of the Self-Replicating RNA

DNA templates encoding a self-replicating RNAs can be produced in competent Escherichia coli cells transformed with a DNA plasmid. Individual bacterial colonies can be isolated and the resultant plasmid DNA amplified in E. coli cultures. Following fermentation, the plasmid DNA can be isolated using Maxiprep DNA kit and linearized by restriction digest. Restriction enzymes can then be removed using phenol/chloroform extraction and ethanol precipitation.

mRNA can be made by in vitro transcription from the linearized DNA template using a T7 RNA polymerase. Subsequently, the DNA template can be removed by DNase digestion. Enzymatic capping can be performed with Cap0 to provide functional mRNA. The resultant mRNA can then be purified and resuspended in nuclease-free water.

### Example 2: In Vitro Characterisation of the Self-Replicating RNA

The self-replicating RNAs produced in Example 1 can then be assessed for expression of the genes of interest that are expressed in the form of an antigen.

Two-fold serial dilutions of unformulated (naked) or LNP-formulated self-amplifying mRNA constructs can be either electroporated or transfected into a Baby Hamster Kidney (BHK) cell line. After about 17-19 hrs, cells can be harvested and stained for either HA, NA, NS1, NP or M1 antigen expression using anti-HA, anti-NA, anti-NS1, anti-NP or anti-M1 antibodies. The number of cells positive for antigen expression and the mean fluorescence intensities (MFIs) can be measured by FACS. Data are analysed to calculate the specific potency values (the probability of successful transfection per unit of mass of RNA) and the MFI generated.

In vitro activity and potency of unformulated RNA and LNPs can be determined by FACs based on antigen co-expression and expressed in read-outs such as FACS potency, encapsulation efficiency, SAM recovery, size, PDI, Zeta potential, conductivity, concentration and endotoxin levels.

### Antibody Responses

To assess antibody responses, serum can be collected at the end of study and tested by microneutralization assays and hemagglutination inhibition assay.

For all serological assays sera can be treated in the same way, with Vibrio cholerae neuraminidase, also known as receptor-destroying enzyme (RDE) (Denka Seiken Co. Ltd., Tokyo, Japan) and diluted to a starting dilution of 1:10 with PBS. Sheep serum to H5N1 virus (FDA/CBER Kensington lot nu. H5-Ag-1115) can be used as positive control sera.

### Microneutralization Assays

Microneutralization assays, short and long form are performed in a qualified mammalian cell line (proprietary 33016-PF Madin-Darby Canine Kidney (MDCK)).

### Microneutralization Assay Short Form (MN Assay SF)

Virus fluorescent focus-based microneutralization (FFA MN) assay can be performed using an in house developed protocol. RDE treated test mouse samples and positive control sera can be heat inactivated, diluted to a starting dilution of 1:40 with PBS, and fourfold serial diluted using the U-Bottom 96 well plate (BD Falcon) in neutralization medium (comprised of minimum essential medium D-MEM (GIBCO), supplemented with 1% BSA (Rockland, BSA-30), 100 U/mL penicillin and 100 μg/mL streptomycin (GIBCO)). A/turkey/Turkey/i/2005 (H5N1) virus can be diluted to ~1,000-1,500 fluorescent focus-forming units (FFU)/well (20,000-30,000 FFU/mL) in neutralization medium and added in a 1:1 ratio to diluted serum.

After incubation for 2 h at 37° C., 5% CO₂, plates (Half Area 96 well plate, Corning) containing MDCK 33016-PF cells can be inoculated with this mixture and incubated overnight for 16-18 h at 37° C. with 5% CO₂. MDCK 33016-PF cells are seeded as 3.0 E4/well (3.0 E6/plate) at 6-8 h earlier in the cell growth medium (comprised of D-MEM, supplemented with 10% HyClone fetal bovine serum—FBS (Gibco), 100 U/mL penicillin and 100 μg/mL streptomycin). Following the overnight incubation and prior to immunostaining, cells can then be fixed with cold mixture of acetone and methanol.

The virus can then be visualized using separate 1 h incubations at room temperature of monoclonal antibodies specific to the virus proteins of interest and Alexa Fluor 488 Goat Anti-Mouse IgG (H+L) Ab (Invitrogen cat. no. A11001) diluted in PBS buffer containing 0.05% tween-20 (Sigma) and 2% BSA (Fraction V, Calbiochem, 2960, 1194C175). Viral protein can be quantified by a CTL Immunospot analyzer (Cellular Technology Limited, Shaker Heights, Cleveland, OH), using a fluorescein isothiocyanate (FITC) fluorescence filter set with excitation and emission wavelengths of 482 and 536 nm. Fluorescent foci can be enumerated by use of software Immunospot 7.0.12.1 professional analyzer DC, using a custom analysis module.

### Microneutralization Assay Long Form (MN Assay LF)

MN assay LF is performed using an in house developed protocol. RDE treated test mouse samples and positive control sera are heat inactivated, diluted to a starting dilution of 1:40 with PBS, and twofold serial diluted using the U-Bottom 96 well plate (BD Falcon) in neutralization medium (comprised of the 30% spent growth media (Irvine Scientific) and 70% infective media (protein free media—33016 MDCK PFM; GIBCO) supplemented with 100 U/mL penicillin, 100 μg/mL streptomycin (GIBCO), and 0.33 ug/mL TPCK-trypsin (TPCK treated, Tosyl phenylalanyl chloromethyl ketone, Sigma). A virus of interest is diluted to 100TCID (tissue culture infectious dose) per well in neutralization medium and added in a 1:1 ratio to diluted serum. Serially pre-diluted serum samples are incubated with the virus and allowed to react for 1 h at 37° C., 5% CO₂. In the inoculation step, plates (Cell Culture 96-well plate, Costar) containing MDCK 33016-PF cells are seeded at 3.0^{E4}/well (3.0^{E6}/plate) a day before in the antibiotic free cell growth medium (Irvine Scientific) and washed with sterile PBS, then infected with this mixture and incubated for 1 h at 37° C. with 5% CO₂. Infection is stopped by aspiration of antibody/virus mixture and cells washed with sterile PBS, inoculated with neutralizing media (100 ul/well) containing twofold serially diluted antibodies and then incubated for 5 days at 37° C. with 5% CO₂. In the final “read-out” step, detection of virus is performed by quantification of the virus using 0.5% turkey red blood cells (Lampire Biological Laboratories). The absence of infectivity constitutes a positive neutralization reaction and indicates the presence of virus-specific antibodies in the serum sample.

### Hemagglutination Inhibition (HAI) Assay

A HAI assay can be performed as previously described (WHO (2011) Manual for the laboratory diagnosis and virological surveillance of influenza: WHO Press, World Health Organization, 20 Avenue Appia, 1211 Geneva 27, Switzerland). Briefly, RDE treated test mouse samples and positive control sera are heat inactivated, diluted to a starting dilution of 1:10 with PBS, and twofold serial diluted samples (25 μl) are incubated with equal volumes of viruses (4 hemagglutinating units [HAU]) of A/turkey/Turkey/1/2005 (H5N1) at room temperature (RT) for 30 minutes. Then, an equal volume of 0.5% turkey red blood cells (Lampire Biological Laboratories) is added and incubated at RT for 30 minutes. The HAI titer can be expressed as the reciprocal of the highest dilution of the samples inhibiting hemagglutination.

### Example 3: Self-Replicating RNA Induces Cell-Mediated Immune Responses

The self-replicating RNAs are assessed for their ability to induce antigen specific T cell responses. Antigen-specific T cell detection is performed on splenic cultures. Briefly, splenocytes are dissociated in dissociation solution (MACS BSA stock 1:20 with autoMACS rinsing solution) and concentrated at 4^{E7} cells/ml. Briefly, splenocyte cultures are established in 96 well plates in T cell medium containing RPMI, NEAA, pen/strep and βME) and cultured at 37° C./5% CO₂. Anti-CD28 (clone 37.51; BD Biosciences #553294) and anti-CD107a (clone #1D4B; Biolegend #121618) are added to each well. Cell cultures are either stimulated or unstimulated. To stimulate cultures NA pep mix (JPT Peptide Technologies GmbH; PM-INFA-NATur), HA pep mix (JPT Peptide Technologies GmbH; PM-INFA-HAlndo) is added. Following 2 hours of stimulation, Golgi Plug (with brefeldin A; BD Biosciences #555029) is added to each well. Cells are incubated at 37° C. for a total of 6 hours after which the cells are transferred to 4° C. and stored overnight.

Antigen-specific T cell responses are determined using flow cytometry. Briefly, Fc block mixture (clone 2.4G2; BD Biosciences #553142) is added to each well, followed by extracellular stain (comprising Brilliant stain buffer plus (BD Biosciences #566385), ICOS BV711 (clone C398.4A; Biolegend #313548), CD44 BUV395 (clone IM7; BD Biosciences #740215), CD3 BV786 (clone 145-2C11; BD Biosciences #564379), CD4 APC-H7 (clone GK1.5, BD Biosciences #560181), CD8 AF700 (clone 53-6.7, BD Biosciences #557959) and staining buffer). Cells are stained with UltraComp eBeads (eBiosciences #01-222-42) according to the manufacturer's protocol and incubated at 4° C. for 30 mins, protected from the light. Cells are washed with staining buffer, centrifuged, resuspended in staining buffer and data acquired using a flow cytometer.

### IgG Subclass

To characterize the type of immune response generated, i.e. Th1 vs Th2 type responses, the S specific IgG1 and IgG2a IgG subclasses can be evaluated by ELISA. In addition, the ratio of IgG1/IgG2a antibodies can also be assessed.

### Example 4: Protective Effect of Immunization with Self-Replicating RNAs

To evaluate the protective effect of immunization, hamsters can be immunized with vaccines described herein at doses of 3 μg RNA/hamster or 0.3 μg RNA/hamster at Day 1 and Day 22. All animals can be challenged 28 days post the second immunization with a suitable virus intranasally and sacrificed 4 days later, where lung and nasal turbinates are collected for infectious virus measured in lungs and nasal turbinates.

To evaluate protection of lungs from virus infection, average virus recovery from lungs are compared for hamsters immunized with vaccines of interest and control hamsters immunized by PBS.

1. A self-replicating RNA comprising a first nucleotide sequence encoding an antigen operably linked to a regulatory element, and a second nucleotide sequence encoding a chemoattractant operably linked to a regulatory element.

2. The self-replicating RNA of claim 1, wherein the chemoattractant is selected from the group consisting of chemerin, C-X-C motif chemokine ligand 9 (CXCL9), C-X-C motif chemokine ligand 10 (CXCL10) and C-X-C motif chemokine ligand 11 (CXCL11).

3. The self-replicating RNA of claim 1 or 2, wherein the self-replicating RNA comprises in 5′ to 3′ order:
a) the nucleotide sequence encoding an antigen operably linked to a regulatory element; and
b) the nucleotide sequence encoding a chemoattractant operably linked to a regulatory element.

4. The self-replicating RNA of claim 1 or 2, wherein the self-replicating RNA comprises in 5′ to 3′ order:
a) the nucleotide sequence encoding a chemoattractant operably linked to a regulatory element; and
b) the nucleotide sequence encoding an antigen operably linked to a regulatory element.

5. The self-replicating RNA of any one of claims 1 to 4, wherein the regulatory element is selected from the group consisting of a promoter, an IRES and a Kozak consensus sequence.

6. The self-replicating RNA of claim 5, wherein the self-replicating RNA comprises in 5′ to 3′ order:
a) the nucleotide sequence encoding an antigen operably linked to a SG promoter; and
b) the nucleotide sequence encoding a chemoattractant operably linked to a regulatory element selected from the group consisting of a SG promoter and an internal ribosome entry site (IRES).

7. The self-replicating RNA of claim 5, wherein the self-replicating RNA comprises in 5′ to 3′ order:
a) the nucleotide sequence encoding a chemoattractant operably linked to a SG promoter; and
b) the nucleotide sequence encoding an antigen operably linked to a regulatory element selected from the group consisting of a SG promoter and an internal ribosome entry site (IRES).

8. The self-replicating RNA of claim 5, wherein the promoter is a subgenomic (SG) promoter.

9. The self-replicating RNA of claim 8, wherein the SG promoter is a minimal SG promoter or an extended SG promoter.

10. The self-replicating RNA of claim 9, wherein the extended SG promoter is extended at the 5′ end with nucleotides occurring in a sequence encoding a non-structural protein of an RNA virus.

11. The self-replicating RNA of claim 9, wherein the minimal SG promoter is encoded by a sequence set forth in SEQ ID NO: 32.

12. The self-replicating RNA of claim 9, wherein the extended SG promoter is encoded by a sequence set forth in SEQ ID NOs: 33, 34, 36, 46, 51 or 52.

13. The self-replicating RNA of claim 5, wherein the nucleotide sequence encoding the chemoattractant is operably linked to an IRES located 5′ to the nucleotide sequence encoding the chemoattractant.

14. The self-replicating RNA of claim 5 or 13, wherein the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (eIF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1 a (FGF1A), GTX, or a combination thereof.

15. The self-replicating RNA of claim 14, wherein the EMCV IRES is a wild-type IRES encoded by a sequence set forth in SEQ ID NO: 35.

16. The self-replicating RNA of any one of claims 1 to 15, wherein the self-replicating RNA is a monocistronic self-replicating RNA.

17. The self-replicating RNA of any one of claims 1 to 15, wherein the self-replicating RNA is a polycistronic self-replicating RNA.

18. The self-replicating RNA of any one of claims 1 to 17, wherein the antigen is selected from the group consisting of influenza virus, respiratory syncytial virus, parainfluenza viruses, metapneumovirus, rhinovirus, coronaviruses, adenoviruses and bocaviruses.

19. The self-replicating RNA of claim 2, wherein the chemoattractant is chemerin having a nucleotide sequence with at least about 70%, at least about 75%, at least about 80%, at least about 85%, at least about 90% at least about 95%, at least about 96%, at least about 97%, at least about 98% or at least about 99% or about 100% sequence identity to any one of SEQ ID NOs: 10, 13, 16 or 19.

20. The self-replicating RNA of claim 19, wherein the chemerin nucleotide has a sequence according to SEQ ID NO: 19.

21. The self-replicating RNA of claim 2, wherein the chemoattractant is selected from the group consisting of CXCL9, CXCL10 and CXCL11 having a polynucleotide sequence with at least about 70%, at least about 75%, at least about 80%, at least about 85%, at least about 90% at least about 95%, at least about 96%, at least about 97%, at least about 98%, at least about 99% or about 100% identity to SEQ ID NOs: 1, 4 or 7 respectively.

22. The self-replicating RNA of any one of claims 1 to 21, wherein the self-replicating RNA is from an alphavirus.

23. The self-replicating RNA of claim 22, wherein the alphavirus is selected from the group consisting of Semliki Forest virus (SFV), Sindbis virus (SIN), and Venezuelan equine encephalitis virus (VEEV) and combinations thereof.

24. The self-replicating RNA of claim 23, wherein the alphavirus is a VEEV.

25. An immunogenic composition comprising the self-replicating RNA of any one of claims 1 to 24.

26. The immunogenic composition of claim 25, comprising a plurality of self-replicating RNAs of any one of claims 1 to 24, wherein each self-replicating RNA encodes different polypeptide sequences.

27. The immunogenic composition of claim 25, comprising a plurality of self-replicating RNAs of any one of claims 1 to 24, wherein each self-replicating RNA encodes the same polypeptide sequence.

28. The immunogenic composition of any one of claims 25 to 27, wherein the immunogenic composition further comprises a chemoattractant polypeptide, optionally selected from the group consisting of chemerin, CXCL9, CXCL10 and CXCL11.

29. An immunogenic composition comprising:
a) a self-replicating RNA comprising a first nucleotide sequence encoding an antigen operably linked to a regulatory element; and
b) a chemoattractant polypeptide, optionally selected from the group consisting of chemerin, CXCL9, CXCL10 and CXCL11.

30. The immunogenic composition of any one of claims 25 to 29, further comprising an additional RNA encoding:
(i) one or more antigens;
(ii) one or more immunopotentiating agents;
(iii) one or more chemoattractants; and/or
(iv) one or more targeting molecules.

31. The immunogenic composition of claim 29 or 30, wherein the regulatory element is a promoter, an internal ribosome entry site (IRES) or a Kozak consensus sequence or a combination thereof.

32. The immunogenic composition of claim 31, wherein the promoter is a subgenomic (SG) promoter.

33. The immunogenic composition of claim 32, wherein the SG promoter is a minimal SG promoter or an extended SG promoter.

34. The immunogenic composition of claim 32, wherein the minimal SG promoter is encoded by a sequence set forth in SEQ ID NO: 32.

35. The immunogenic composition of claim 32, wherein the extended SG promoter is encoded by a sequence set forth in SEQ ID NOs: 33, 34, 36, 46, 51 or 52.

36. The immunogenic composition of claim 31, wherein the nucleotide sequence encoding a chemoattractant is operably linked to an IRES located 5′ to the nucleotide sequence encoding the chemoattractant.

37. The immunogenic composition of claim 36, wherein the IRES is an IRES from encephalomyocarditis virus (EMCV), poliovirus (PV), human enterovirus, foot-and-mouth disease virus (FMDV), hepatitis C virus (HCV), classical swine fever virus (CSFV), murine leukemia virus (MLV), simian immunodeficiency virus (SIV), Eukaryotic translation initiation factor 4G (eIF4G), Death-associated protein 5 (DAP5), cellular Myc (c-Myc), NF-κB-repressing factor (NRF), vascular endothelial growth factor (VEGF), fibroblast growth factor (FGF-2), platelet-derived growth factor B (PDGF B), Antennapedia, X-linked inhibitor of apoptosis (XIAP or Apaf-1), immunoglobulin heavy-chain binding protein BiP, or fibroblast growth factor 1 a (FGF1A), GTX, or a combination thereof.

38. The immunogenic composition of claim 37, wherein the EMCV IRES is a wild-type IRES encoded by a sequence set forth in SEQ ID NO: 35.

39. The immunogenic composition of any one of claims 29 to 38, wherein the antigen is selected from the group consisting of influenza virus, respiratory syncytial virus, parainfluenza viruses, metapneumovirus, rhinovirus, coronaviruses, adenoviruses and bocaviruses.

40. The immunogenic composition of any one of claims 29 to 39, wherein the chemoattractant polypeptide is selected from the group consisting of chemerin, C-X-C motif chemokine ligand 9 (CXCL9), C-X-C motif chemokine ligand 10 (CXCL10) and C-X-C motif chemokine ligand 11 (CXCL11).

41. The immunogenic composition of claim 40, wherein the chemoattractant polypeptide is chemerin comprising an amino acid sequence having at least about 70%, at least about 75%, at least about 80%, at least about 85%, at least about 90% at least about 95%, at least about 96%, at least about 97%, at least about 98%, at least about 99% or about 100% sequence identity to any one of SEQ ID NOs: 12, 15, 18 or 21.

42. The immunogenic composition of claim 41, wherein the chemerin polypeptide has an amino acid sequence set forth in SEQ ID NO: 21.

43. The immunogenic composition of claim 40, wherein the chemoattractant polypeptide is CXCL9, CXCL10 or CXCL11 comprising an amino acid sequence having at least about 70%, at least about 75%, at least about 80%, at least about 85%, at least about 90% at least about 95%, at least about 96%, at least about 97%, at least about 98%, at least about 99% or about 100% identity to any one of SEQ ID NO: 3, 6 or 9 respectively.

44. The immunogenic composition of any one of claims 29 to 43, wherein the self-replicating RNA is from an alphavirus.

45. The immunogenic composition of claim 44, wherein the alphavirus is selected from the group consisting of Semliki Forest virus (SFV), Sindbis virus (SIN), and Venezuelan equine encephalitis virus (VEE) and combinations thereof.

46. The immunogenic composition of claim 45, wherein the alphavirus is VEEV and the antigen is a H5 antigen from influenza, preferably having a sequence having at least about 70%, at least about 75%, at least about 80%, at least about 85%, at least about 90% at least about 95%, at least about 96%, at least about 97%, at least about 98%, at least about 99% or 100% sequence identity to SEQ ID NO: 49.

47. A pharmaceutical composition comprising an immunogenic composition of any one of claims 25 to 46 and a pharmaceutically acceptable carrier.

48. The pharmaceutical composition of claim 47, further comprising a lipid nanoparticle (LNP), a polymeric microparticle or an oil-in-water emulsion.

49. The pharmaceutical composition of claim 48, wherein the self-replicating RNA is encapsulated in, bound to or adsorbed on a LNP, a polymeric microparticle or an oil-in-water emulsion.

50. The pharmaceutical composition of any one of claims 47 to 49, wherein each RNA is formulated together in the LNP.

51. The pharmaceutical composition of any one of claims 47 to 50, wherein each RNA is formulated separately in the LNP.

52. The immunogenic composition of any one of claims 25 to 46 or the pharmaceutical composition of any one of claims 48 to 51, for use as a vaccine.

53. A vaccine comprising the pharmaceutical composition of any one of claims 48 to 51 or the immunogenic composition of any one of claims 25 to 46.

54. A polynucleotide encoding the self-replicating RNA of any one of claims 1 to 24.

55. The polynucleotide of claim 54, wherein the polynucleotide is a recombinant DNA.

56. The polynucleotide of claim 55, wherein the recombinant DNA is a plasmid.

57. The polynucleotide of claim 56, wherein the plasmid comprises a sequence set forth in SEQ ID NO: 50.

58. A polynucleotide comprising:
a) a first nucleotide sequence encoding an antigen operably linked to a regulatory element; and
b) a second nucleotide sequence encoding a chemoattractant operably linked to a regulatory element selected from the group consisting of a SG promoter and an internal ribosome entry site (IRES),
optionally wherein the chemoattractant is selected from the group consisting of chemerin, C-X-C motif chemokine ligand 9 (CXCL9), C-X-C motif chemokine ligand 10 (CXCL10) and C-X-C motif chemokine ligand 11 (CXCL11).

59. The polynucleotide of claim 58, wherein the polynucleotide comprises, in order from 5′ to 3′:
a) the first nucleotide sequence encoding an antigen operably linked to a regulatory element; and
b) the second nucleotide sequence encoding a chemoattractant operably linked to an IRES or a SG promoter.

60. A conventional mRNA (cRNA) comprising:
a) a first nucleotide sequence encoding an antigen operably linked to a regulatory element; and
b) a second nucleotide sequence encoding a chemoattractant operably linked to a regulatory element selected from the group consisting of a SG promoter and an internal ribosome entry site (IRES),
optionally wherein the chemoattractant is selected from the group consisting of chemerin, C-X-C motif chemokine ligand 9 (CXCL9), C-X-C motif chemokine ligand 10 (CXCL10) and C-X-C motif chemokine ligand 11 (CXCL11).

61. The cRNA of claim 60, wherein the cRNA comprises, in order from 5′ to 3′:
a) the first nucleotide sequence encoding an antigen operably linked to a regulatory element; and
b) the second nucleotide sequence encoding a chemoattractant operably linked to an IRES or a SG promoter.

62. The polynucleotide of any one of claims 54 to 59, or the cRNA of claim 60 or 61, wherein the first nucleotide sequence is operably linked to a regulatory element selected from the group consisting of a Kozak consensus sequence, an IRES, a SG promoter and combinations thereof.

63. A method of treating or preventing or delaying progression of a disease or condition in a subject in need thereof, the method comprising administering the pharmaceutical composition of any one of claims 47 to 51, the immunogenic composition any one of claims 25 to 46 or the vaccine of claim 53 to the subject.

64. Use of the pharmaceutical composition of any one of claims 47 to 51, the immunogenic composition any one of claims 25 to 46 or the vaccine of claim 53 in the manufacture of a medicament for treating or preventing or delaying progression of a disease or condition in a subject in need thereof.

65. The pharmaceutical composition of any one of claims 47 to 51, the immunogenic composition any one of claims 25 to 46 or the vaccine of claim 53 for use in treating or preventing or delaying progression of a disease or condition in a subject in need thereof.

66. A method of inducing an immune response in a subject, the method comprising administering the pharmaceutical composition of any one of claims 47 to 51, the immunogenic composition any one of claims 25 to 46 or the vaccine of claim 53 to a subject in need thereof.

67. Use of the pharmaceutical composition of any one of claims 47 to 51, the immunogenic composition any one of claims 25 to 46 or the vaccine of claim 53 in the manufacture of a medicament for inducing an immune response in a subject in need thereof.

68. The pharmaceutical composition of any one of claims 47 to 51, the immunogenic composition any one of claims 25 to 46 or the vaccine of claim 53 for use in inducing an immune response in a subject in need thereof.

69. The method of claim 66, the use of claim 67, or the self-replicating RNA, pharmaceutical composition, immunogenic composition or vaccine for use of claim 68, wherein the immune response is a humoral and/or a cell-mediated immune response.

70. A method for reducing viral load in a subject comprising administering pharmaceutical composition of any one of claims 47 to 51, the immunogenic composition any one of claims 25 to 46 or the vaccine of claim 53 to the subject in need thereof.

71. Use of the pharmaceutical composition of any one of claims 47 to 51, the immunogenic composition any one of claims 25 to 46 or the vaccine of claim 53 in the preparation of a medicament for reducing viral load in a subject in need thereof.

72. The pharmaceutical composition of any one of claims 47 to 51, the immunogenic composition any one of claims 25 to 46 or the vaccine of claim 53 for use in reducing viral load in a subject in need thereof.

73. The method of any one of claims 63, 66, 69 or 70, the use of any one of claims 64, 67, 69 or 71, or the pharmaceutical composition, immunogenic composition or vaccine for use of any one of claims 65, 68, 69 or 72, wherein the subject is a human of 18 years of age or older.

74. The method of any one of claims 63, 66, 69 or 70, the use of any one of claims 64, 67, 69 or 71, or the pharmaceutical composition, immunogenic composition or vaccine for use of any one of claims 65, 68, 69 or 72, wherein the vaccine or composition is administered in a one dose regimen.

75. The method of any one of claims 63, 66, 69 or 70, the use of any one of claims 64, 67, 69 or 71, or the pharmaceutical composition, immunogenic composition or vaccine for use of any one of claims 65, 68, 69 or 72, wherein the vaccine or composition is administered in a two, three or four dose regimen, wherein the doses are administered about 1, 2 or 3 months apart.

76. A kit comprising:
(a) a self-replicating RNA of any one of claims 1 to 24, pharmaceutical composition of any one of claims 47 to 51, the immunogenic composition any one of claims 25 to 46 or the vaccine of claim 53;
(b) instructions for use thereof; and optionally
(c) a pharmaceutically acceptable carrier, excipient or diluent.


SEQUENCE LISTING (52 sequences)

SEQ ID NO: 1 - RNA, 375 nt, Homo sapiens
  source 1..375: mol_type = mRNA
atgaagaagagcggcgtgctgttcctgctgggcatcatcctgctggtgctgatcggcgtgcagggcacccccgtggtgaggaagggcaggtgcagctgcatcagcaccaaccagggcaccatccacctgcagagcctgaaggacctgaagcagttcgcccccagccccagctgcgagaagatcgagatcatcgccaccctgaagaacggcgtgcagacctgcctgaaccccgacagcgccgacgtgaaggagctgatcaagaagtgggagaagcaggtgagccagaagaagaagcagaagaacggcaagaagcaccagaagaagaaggtgctgaaggtgaggaagagccagaggagcaggcagaagaagaccacc

SEQ ID NO: 2 - DNA, 375 bp, Homo sapiens
  source 1..375: mol_type = genomic DNA
atgaagaagagcggcgtgctgttcctgctgggcatcatcctgctggtgctgatcggcgtgcagggcacccccgtggtgaggaagggcaggtgcagctgcatcagcaccaaccagggcaccatccacctgcagagcctgaaggacctgaagcagttcgcccccagccccagctgcgagaagatcgagatcatcgccaccctgaagaacggcgtgcagacctgcctgaaccccgacagcgccgacgtgaaggagctgatcaagaagtgggagaagcaggtgagccagaagaagaagcagaagaacggcaagaagcaccagaagaagaaggtgctgaaggtgaggaagagccagaggagcaggcagaagaagaccacc

SEQ ID NO: 3 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 4 - RNA, 294 nt, Homo sapiens
  source 1..294: mol_type = mRNA
atgaaccagaccgccatcctgatctgctgcctgatcttcctgaccctgagcggcatccagggcgtgcccctgagcaggaccgtgaggtgcacctgcatcagcatcagcaaccagcccgtgaaccccaggagcctggagaagctggagatcatccccgccagccagttctgccccagggtggagatcatcgccaccatgaagaagaagggcgagaagaggtgcctgaaccccgagagcaaggccatcaagaacctgctgaaggccgtgagcaaggagaggagcaagaggagcccc

SEQ ID NO: 5 - DNA, 294 bp, Homo sapiens
  source 1..294: mol_type = genomic DNA
atgaaccagaccgccatcctgatctgctgcctgatcttcctgaccctgagcggcatccagggcgtgcccctgagcaggaccgtgaggtgcacctgcatcagcatcagcaaccagcccgtgaaccccaggagcctggagaagctggagatcatccccgccagccagttctgccccagggtggagatcatcgccaccatgaagaagaagggcgagaagaggtgcctgaaccccgagagcaaggccatcaagaacctgctgaaggccgtgagcaaggagaggagcaagaggagcccc

SEQ ID NO: 6 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 7 - RNA, 282 nt, Homo sapiens
  source 1..282: mol_type = mRNA
atgagcgtgaagggcatggccatcgccctggccgtgatcctgtgcgccaccgtggtgcagggcttccccatgttcaagaggggcaggtgcctgtgcatcggccccggcgtgaaggccgtgaaggtggccgacatcgagaaggccagcatcatgtaccccagcaacaactgcgacaagatcgaggtgatcatcaccctgaaggagaacaagggccagaggtgcctgaaccccaagagcaagcaggccaggctgatcatcaagaaggtggagaggaagaacttc

SEQ ID NO: 8 - DNA, 282 bp, Homo sapiens
  source 1..282: mol_type = genomic DNA
atgagcgtgaagggcatggccatcgccctggccgtgatcctgtgcgccaccgtggtgcagggcttccccatgttcaagaggggcaggtgcctgtgcatcggccccggcgtgaaggccgtgaaggtggccgacatcgagaaggccagcatcatgtaccccagcaacaactgcgacaagatcgaggtgatcatcaccctgaaggagaacaagggccagaggtgcctgaaccccaagagcaagcaggccaggctgatcatcaagaaggtggagaggaagaacttc

SEQ ID NO: 9 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 10 - RNA, 489 nt, Homo sapiens
  source 1..489: mol_type = mRNA
atgaggaggctgctgatccccctggccctgtggctgggcgccgtgggcgtgggcgtggccgagctgaccgaggcccagaggaggggcctgcaggtggccctggaggagttccacaagcacccccccgtgcagtgggccttccaggagaccagcgtggagagcgccgtggacacccccttccccgccggcatcttcgtgaggctggagttcaagctgcagcagaccagctgcaggaagagggactggaagaagcccgagtgcaaggtgaggcccaacggcaggaagaggaagtgcctggcctgcatcaagctgggcagcgaggacaaggtgctgggcaggctggtgcactgccccatcgagacccaggtgctgagggaggccgaggagcaccaggagacccagtgcctgagggtgcagagggccggcgaggacccccacagcttctacttccccggccagttcgccttcagcaaggccctgcccaggagc

SEQ ID NO: 11 - DNA, 489 bp, Homo sapiens
  source 1..489: mol_type = genomic DNA
atgaggaggctgctgatccccctggccctgtggctgggcgccgtgggcgtgggcgtggccgagctgaccgaggcccagaggaggggcctgcaggtggccctggaggagttccacaagcacccccccgtgcagtgggccttccaggagaccagcgtggagagcgccgtggacacccccttccccgccggcatcttcgtgaggctggagttcaagctgcagcagaccagctgcaggaagagggactggaagaagcccgagtgcaaggtgaggcccaacggcaggaagaggaagtgcctggcctgcatcaagctgggcagcgaggacaaggtgctgggcaggctggtgcactgccccatcgagacccaggtgctgagggaggccgaggagcaccaggagacccagtgcctgagggtgcagagggccggcgaggacccccacagcttctacttccccggccagttcgccttcagcaaggccctgcccaggagc

SEQ ID NO: 12 - protein, 163 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 13 - RNA, 429 nt, Homo sapiens
  source 1..429: mol_type = mRNA
gagctgaccgaggcccagaggaggggcctgcaggtggccctggaggagttccacaagcacccccccgtgcagtgggccttccaggagaccagcgtggagagcgccgtggacacccccttccccgccggcatcttcgtgaggctggagttcaagctgcagcagaccagctgcaggaagagggactggaagaagcccgagtgcaaggtgaggcccaacggcaggaagaggaagtgcctggcctgcatcaagctgggcagcgaggacaaggtgctgggcaggctggtgcactgccccatcgagacccaggtgctgagggaggccgaggagcaccaggagacccagtgcctgagggtgcagagggccggcgaggacccccacagcttctacttccccggccagttcgccttcagcaaggccctgcccaggagc

SEQ ID NO: 14 - DNA, 429 bp, Homo sapiens
  source 1..429: mol_type = genomic DNA
gagctgaccgaggcccagaggaggggcctgcaggtggccctggaggagttccacaagcacccccccgtgcagtgggccttccaggagaccagcgtggagagcgccgtggacacccccttccccgccggcatcttcgtgaggctggagttcaagctgcagcagaccagctgcaggaagagggactggaagaagcccgagtgcaaggtgaggcccaacggcaggaagaggaagtgcctggcctgcatcaagctgggcagcgaggacaaggtgctgggcaggctggtgcactgccccatcgagacccaggtgctgagggaggccgaggagcaccaggagacccagtgcctgagggtgcagagggccggcgaggacccccacagcttctacttccccggccagttcgccttcagcaaggccctgcccaggagc

SEQ ID NO: 15 - protein, 143 aa, Homo sapiens
ELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 16 - RNA, 408 nt, Homo sapiens
  source 1..408: mol_type = mRNA
ctgaccgaggcccagaggaggggcctgcaggtggccctggaggagttccacaagcacccccccgtgcagtgggccttccaggagaccagcgtggagagcgccgtggacacccccttccccgccggcatcttcgtgaggctggagttcaagctgcagcagaccagctgcaggaagagggactggaagaagcccgagtgcaaggtgaggcccaacggcaggaagaggaagtgcctggcctgcatcaagctgggcagcgaggacaaggtgctgggcaggctggtgcactgccccatcgagacccaggtgctgagggaggccgaggagcaccaggagacccagtgcctgagggtgcagagggccggcgaggacccccacagcttctacttccccggccagttcgccttcagc

SEQ ID NO: 17 - DNA, 429 bp, Homo sapiens; identical to SEQ ID NO: 14
  source 1..429: mol_type = genomic DNA

SEQ ID NO: 18 - protein, 136 aa, Homo sapiens
LTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFS

SEQ ID NO: 19 - RNA, 471 nt, Homo sapiens
  source 1..471: mol_type = mRNA
atgcgccgcctgctgattccgctggcgctgtggctgggcgcggtgggcgtgggcgtggcggaactgaccgaagcgcagcgccgcggcctgcaggtggcgctggaagaatttcataaacatccgccggtgcagtgggcgtttcaggaaaccagcgtggaaagcgcggtggataccccgtttccggcgggcatttttgtgcgcctggaatttaaactgcagcagaccagctgccgcaaacgcgattggaaaaaaccggaatgcaaagtgcgcccgaacggccgcaaacgcaaatgcctggcgtgcattaaactgggcagcgaagataaagtgctgggccgcctggtgcattgcccgattgaaacccaggtgctgcgcgaagcggaagaacatcaggaaacccagtgcctgcgcgtgcagcgcgcgggcgaagatccgcatagcttttattttccgggccagtttgcgtttagc

SEQ ID NO: 20 - DNA, 471 bp, Homo sapiens
  source 1..471: mol_type = genomic DNA
atgcgccgcctgctgattccgctggcgctgtggctgggcgcggtgggcgtgggcgtggcggaactgaccgaagcgcagcgccgcggcctgcaggtggcgctggaagaatttcataaacatccgccggtgcagtgggcgtttcaggaaaccagcgtggaaagcgcggtggataccccgtttccggcgggcatttttgtgcgcctggaatttaaactgcagcagaccagctgccgcaaacgcgattggaaaaaaccggaatgcaaagtgcgcccgaacggccgcaaacgcaaatgcctggcgtgcattaaactgggcagcgaagataaagtgctgggccgcctggtgcattgcccgattgaaacccaggtgctgcgcgaagcggaagaacatcaggaaacccagtgcctgcgcgtgcagcgcgcgggcgaagatccgcatagcttttattttccgggccagtttgcgtttagc

SEQ ID NO: 21 - protein, 157 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFS

SEQ ID NO: 22 - RNA, 420 nt, Mus musculus
  source 1..420: mol_type = mRNA
gagctgagcgagacccagaggaggagcctgcaggtggccctggaggagttccacaagcacccccccgtgcagctggccttccaggagatcggcgtggacagggccgaggaggtgctgttcagcgccggcaccttcgtgaggctggagttcaagctgcagcagaccaactgccccaagaaggactggaagaagcccgagtgcaccatcaagcccaacggcaggaggaggaagtgcctggcctgcctgaagatggaccccaagggcaagatcctgggcaggatcgtgcactgccccatcctgaagcagggcccccaggacccccaggagctgcagtgcatcaagatcgcccaggccggcgaggacccccacggctacttcctgcccggccagttcgccttcagcagggccctgaggaccaag

SEQ ID NO: 23 - DNA, 420 bp, Mus musculus
  source 1..420: mol_type = genomic DNA
gagctgagcgagacccagaggaggagcctgcaggtggccctggaggagttccacaagcacccccccgtgcagctggccttccaggagatcggcgtggacagggccgaggaggtgctgttcagcgccggcaccttcgtgaggctggagttcaagctgcagcagaccaactgccccaagaaggactggaagaagcccgagtgcaccatcaagcccaacggcaggaggaggaagtgcctggcctgcctgaagatggaccccaagggcaagatcctgggcaggatcgtgcactgccccatcctgaagcagggcccccaggacccccaggagctgcagtgcatcaagatcgcccaggccggcgaggacccccacggctacttcctgcccggccagttcgccttcagcagggccctgaggaccaag

SEQ ID NO: 24 - protein, 140 aa, Mus musculus
ELSETQRRSLQVALEEFHKHPPVQLAFQEIGVDRAEEVLFSAGTFVRLEFKLQQTNCPKKDWKKPECTIKPNGRRRKCLACLKMDPKGKILGRIVHCPILKQGPQDPQELQCIKIAQAGEDPHGYFLPGQFAFSRALRTK

SEQ ID NO: 25 - RNA, 402 nt, Mus musculus
  source 1..402: mol_type = mRNA
gagctgagcgagacccagaggaggagcctgcaggtggccctggaggagttccacaagcacccccccgtgcagctggccttccaggagatcggcgtggacagggccgaggaggtgctgttcagcgccggcaccttcgtgaggctggagttcaagctgcagcagaccaactgccccaagaaggactggaagaagcccgagtgcaccatcaagcccaacggcaggaggaggaagtgcctggcctgcatcaagatggaccccaagggcaagatcctgggcaggatcgtgcactgccccatcctgaagcagggcccccaggacccccaggagctgcagtgcatcaagatcgcccaggccggcgaggacccccacggctacttcctgcccggccagttcgccttcagc

SEQ ID NO: 26 - DNA, 402 bp, Mus musculus
  source 1..402: mol_type = genomic DNA
gagctgagcgagacccagaggaggagcctgcaggtggccctggaggagttccacaagcacccccccgtgcagctggccttccaggagatcggcgtggacagggccgaggaggtgctgttcagcgccggcaccttcgtgaggctggagttcaagctgcagcagaccaactgccccaagaaggactggaagaagcccgagtgcaccatcaagcccaacggcaggaggaggaagtgcctggcctgcatcaagatggaccccaagggcaagatcctgggcaggatcgtgcactgccccatcctgaagcagggcccccaggacccccaggagctgcagtgcatcaagatcgcccaggccggcgaggacccccacggctacttcctgcccggccagttcgccttcagc

SEQ ID NO: 27 - protein, 134 aa, Mus musculus
ELSETQRRSLQVALEEFHKHPPVQLAFQEIGVDRAEEVLFSAGTFVRLEFKLQQTNCPKKDWKKPECTIKPNGRRRKCLACIKMDPKGKILGRIVHCPILKQGPQDPQELQCIKIAQAGEDPHGYFLPGQFAFS

SEQ ID NO: 28 - RNA, 468 nt, Mus musculus
  source 1..468: mol_type = mRNA
atgaagtgcctgctgatcagcctggccctgtggctgggcaccgtgggcaccaggggcaccgagcccgagctgagcgagacccagaggaggagcctgcaggtggccctggaggagttccacaagcacccccccgtgcagctggccttccaggagatcggcgtggacagggccgaggaggtgctgttcagcgccggcaccttcgtgaggctggagttcaagctgcagcagaccaactgccccaagaaggactggaagaagcccgagtgcaccatcaagcccaacggcaggaggaggaagtgcctggcctgcatcaagatggaccccaagggcaagatcctgggcaggatcgtgcactgccccatcctgaagcagggcccccaggacccccaggagctgcagtgcatcaagatcgcccaggccggcgaggacccccacggctacttcctgcccggccagttcgccttcagc

SEQ ID NO: 29 - DNA, 468 bp, Mus musculus
  source 1..468: mol_type = genomic DNA
atgaagtgcctgctgatcagcctggccctgtggctgggcaccgtgggcaccaggggcaccgagcccgagctgagcgagacccagaggaggagcctgcaggtggccctggaggagttccacaagcacccccccgtgcagctggccttccaggagatcggcgtggacagggccgaggaggtgctgttcagcgccggcaccttcgtgaggctggagttcaagctgcagcagaccaactgccccaagaaggactggaagaagcccgagtgcaccatcaagcccaacggcaggaggaggaagtgcctggcctgcatcaagatggaccccaagggcaagatcctgggcaggatcgtgcactgccccatcctgaagcagggcccccaggacccccaggagctgcagtgcatcaagatcgcccaggccggcgaggacccccacggctacttcctgcccggccagttcgccttcagc

SEQ ID NO: 30 - protein, 156 aa, Mus musculus
MKCLLISLALWLGTVGTRGTEPELSETQRRSLQVALEEFHKHPPVQLAFQEIGVDRAEEVLFSAGTFVRLEFKLQQTNCPKKDWKKPECTIKPNGRRRKCLACIKMDPKGKILGRIVHCPILKQGPQDPQELQCIKIAQAGEDPHGYFLPGQFAFS

SEQ ID NO: 31 - protein, 157 aa, Homo sapiens
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFSFAFQGPFY

SEQ ID NO: 32 - DNA, 49 bp, synthetic construct
  source 1..49: mol_type = other DNA
ctctctacggctaacctgaatggactacgacatagtctagtccgccaag

SEQ ID NO: 33 - DNA, 61 bp, synthetic construct
  source 1..61: mol_type = other DNA
gggcccctataactctctacggctaacctgaatggactacgacatagtctagtccgccaag

SEQ ID NO: 34 - DNA, 80 bp, synthetic construct
  source 1..80: mol_type = other DNA
tcattcagctacctgagaggggcccctataactctctacggctaacctgaatggactacgacatagtctagtccgccaag

SEQ ID NO: 35 - DNA, 575 bp, synthetic construct
  source 1..575: mol_type = other DNA
cccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 36 - DNA, 101 bp, synthetic construct
  source 1..101: mol_type = other DNA
actctagctagcagtgttaaatcattcagctacctgagaggggcccctataactctctacggctaacctgaatggactacgacatagtctagtccgccaag

SEQ ID NO: 37-38 - skipped (2 entries)

SEQ ID NO: 39 - DNA, 10 bp, synthetic construct
  source 1..10: mol_type = other DNA
gcatatgact

SEQ ID NO: 40 - DNA, 94 bp, synthetic construct
  source 1..94: mol_type = other DNA
cccctccccgacacggattggctggtgtagtgggcgcggccagagaccacccagcgcccgaccccccctccccacaaacacggggggcgtccct

SEQ ID NO: 41 - DNA, 10 bp, synthetic construct
  source 1..10: mol_type = other DNA
ccccggcgcc

SEQ ID NO: 42 - skipped

SEQ ID NO: 43 - DNA, 26 bp, synthetic construct
  source 1..26: mol_type = other DNA
ccaaaggctcttttcagagccaccca

SEQ ID NO: 44 - DNA, 44 bp, synthetic construct
  source 1..44: mol_type = other DNA
ataggcggcgcatgagagaagcccagaccaattacctacccaaa

SEQ ID NO: 45 - DNA, 319 bp, synthetic construct
  source 1..319: mol_type = other DNA
ccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 46 - DNA, 70 bp, synthetic construct
  source 1..70: mol_type = other DNA
gggcccctataactctctacggctaacctgaatggacctgaatggactacgacatagtctagtccgccaa

SEQ ID NO: 47 - DNA, 36 bp, synthetic construct
  source 1..36: mol_type = other DNA
aaaaaaaaaaaaaaaaaaaaaaaaaaaaaaaaaaaa

SEQ ID NO: 48 - DNA, 9631 bp, synthetic construct
  source 1..9631: mol_type = other DNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaa

SEQ ID NO: 49 - RNA, 9631 nt, synthetic construct
  source 1..9631: mol_type = other RNA
gataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaa

SEQ ID NO: 50 - DNA, 11884 bp, synthetic construct
  source 1..11884: mol_type = other DNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggagaaaatagtgcttcttcttgcaatagtcagccttgttaaaagtgatcagatttgcattggttaccatgcaaacaactcgacagagcaggttgacacaataatggaaaagaacgtcactgttacacacgcccaagacatactggaaaagacacacaacgggaaactctgcgatctagatggagtgaagcctctaattttaagagattgtagtgtagctggatggctcctcgggaacccaatgtgtgacgaattcctcaatgtgccggaatggtcttacatagtggagaagatcaatccagccaatgacctctgttacccagggaatttcaacgactatgaagaactgaaacacctattgagcagaataaaccattttgagaaaattcagatcatccccaaaagttcttggtcagatcatgaagcctcagcaggggtgagctcagcatgtccataccagggaaggtcctccttttttagaaatgtggtatggcttatcaaaaaggacaatgcatacccaacaataaagagaagttacaataataccaaccaagaagatcttttggtattgtgggggattcaccatccaaatgatgcggcagagcagacaaggctctatcaaaacccaactacctatatttccgttgggacatcaacactaaaccagagattggtaccaaaaatagccactagatctaaggtaaacgggcaaagtggaaggatggagttcttttggacaattttaaaaccgaatgatgcaataaactttgagagtaatggaaatttcattgctccagaaaatgcatacaaaattgtcaagaaaggggactcaacaattatgaaaagtgagttggaatatggtaactgcaacaccaagtgtcaaactccaataggggcgataaactctagtatgccattccacaacatccaccctctcaccatcggggaatgccccaaatatgtgaaatcaagcagattagtccttgctactgggctcagaaatagccctcaacgagagacacgaggactatttggagctatagcaggttttatagagggaggatggggaatggtagatggttggtatgggtaccaccatagcaacgagcaggggagtgggtacgctgcagacaaagaatccactcaaaaggcaatagatggagtcaccaataaggtcaactcgatcattgacaaaatgaacactcagtttgaggctgttggaagggaatttaataacttagaaaggagaatagaaaatttaaacaagaagatggaagacggattcctagatgtctggacttataatgctgaacttctggttctcatggaaaatgagagaactctagactttcatgactcaaatgtcaagaacctttacgacaaggtccgactacagcttagggataatgcaaaggagcttggtaacggttgtttcgagttctatcacagatgtgataatgaatgtatggaaagtgtaagaaacggaacgtatgactacccgcagtattcagaagaagcaagattaaaaagagaggaaataagtggagtaaaattggaatcaataggaacttaccaaatactgtcaatttattcaacagtggcgagctccctagcactggcaatcatggtggctggtctatctttatggatgtgctccaatggatcgttacaatgcagaatttgcatttaaggcgcgcccacccagcggccgcccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaggcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 51 - DNA, 80 bp, synthetic construct; identical to SEQ ID NO: 34
  source 1..80: mol_type = other DNA

SEQ ID NO: 52 - DNA, 101 bp, synthetic construct; identical to SEQ ID NO: 36
  source 1..101: mol_type = other DNA